(19) United States
(12) Patent Application Publication
(10) Pub. No.: US 2010/0015654 A1
(43) Pub. Date: Jan. 21, 2010

(54) Negative regulation of NK cell functions by EAT-2, a sap-related adaptor expressed in innate immune cells

(75) Inventors: Andre Veillette, Montreal (CA); Romain Roncagalli, Marseille (FR)

Correspondence Address: MCANDREWS HELD & MALLOY, LTD, 500 WEST MADISON STREET, SUITE 3400, CHICAGO, IL, 60661, US

(73) Assignee: Institut De Recherches Cliniques De Montreal/ I.R.C.M., Montreal, QC (CA)

(21) Appl. No.: 11/914,512

(22) PCT Filed: May 15, 2006
(86) PCT No.: PCT/CA06/00785

§ 371 Date: May 8, 2008

Related U.S. Application Data
(60) Provisional application No. 60/681,081, filed on May 16, 2005.

(51) Int. Cl. C12Q 1/02; C07H 21/02; C12N 5/02

(52) U.S. Cl. 435/29; 536/24.5; 435/375

(57) ABSTRACT

The present invention relates to the identification EAT-2 and ERT as novel therapeutic targets for the modulation of innate immune cell functions. More particularly the present invention describes novel methods for modulating innate immune cells-mediated immune response, useful in the treatment of cancer, infectious diseases as well as autoimmune diseases. The invention also features EAT-2 deficient and overexpressing transgenic animals, screening assays to identify agents that modulate EAT-2 and ERT activity or expression as well as methods of treatments comprising a modulation of NK cells function

### FIELD OF THE INVENTION

The present invention generally relates to methods for the modulation of innate immune cells-mediated immune response. More specifically, the present invention is concerned with novel methods for modulating the functions of innate immune cells such as natural killer cells, dendritic cells and macrophages. These methods are useful, for example, for treating infectious diseases, autoimmune diseases as well as tumours.

### BACKGROUND OF THE INVENTION

Innate immune cells, including natural killer (NK) cells, dendritic cells (DCs) and macrophages, play a critical role in health and notably in protecting humans against cancer and infectious diseases. Moreover, they have been implicated in the pathophysiology of diseases such as auto-immunity. Modulating innate immune cell function by therapeutic means could thus have a significant impact on the treatment of such diseases.

Natural killer cells constitute a population of lymphocytes which represent a very early line of defense against viruses and tumour cells. NK cells can be characterized by the presence of CD56 and CD16 markers and by the absence of CD3 marker. They are involved in a non-specific anti-tumoral immunity of antigens to prevent the establishment of primitive or metastatic tumours. NK cells are capable of recognizing abnormal cells such as cancer cells and virus-infected cells due to their ability to sense expression of normal and abnormal constituents at the surface of other cells. This process is mediated by a large number of receptors present on NK cells. Once they recognize cells as foreign or abnormal (as is the case for cancer cells or virally-infected cells), NK cells destroy these “target” cells by releasing killing enzymes and by recruiting other types of immune cells through the production of substances referred to as “cytokines”.

DCs and macrophages are responsible for recognizing, engulfing and digesting cancer cells and cells infected by pathogens including viruses. Once this has happened, they are able to present small portions (“antigens”) of foreign cell proteins to other immune cells known as T-cells. This process, referred to as antigen presentation, results in the initiation of an immune response specifically directed against the cancer cells or the infected cells. Enhancing DC functions has been proposed to be a potentially useful way to augment the efficacy of vaccination against cancer and infectious diseases.

Natural killer (NK) cells are part of the first line of defense against pathogens and cancer cells¹⁻³. Unlike T- and B-cells, they do not express polymorphic antigen receptors. However, they express several types of stimulatory and inhibitory receptors that recognize ligands on potential target cells. The stimulatory receptors include NKG2D, stimulatory Ly49s, CD16 and natural cytotoxicity receptors, which are associated with subunits that carry “immunoreceptor tyrosine-based activation motifs” (ITAMs) in their cytoplasmic domain. In contrast, the inhibitory receptors include inhibitory Ly49s and KIRs (killer cell immunoglobulin-like receptors), which recognize self-class I major histocompatibility complex (MHC) molecules on potential target cells and contain “immunoreceptor tyrosine-based inhibitory motifs” (ITIMs) in their cytoplasmic region. The balance between the signals delivered by these two types of receptors determines in large part whether or not an NK cell will destroy a potential target cell.

Other types of receptors also control NK cell activity. Amongst these is 2B4 (CD244), a signalling lymphocyte activation molecule (SLAM)-related receptor abundantly expressed in NK cells⁴⁻⁸. By way of its extra-cellular domain, 2B4 interacts with CD48, a member of the CD2 family broadly expressed in immune cells, including NK cells^{9,10}. Several studies provided compelling evidence that 2B4 engagement by CD48 stimulates the ability of NK cells to kill target cells and to produce inflammatory cytokines such as interferon (IFN)-γ^{6,7,11}. However, analyses of 2B4-deficient mice indicated that 2B4 could also be an inhibitory receptor^{12,13}. While the precise molecular basis for these two opposite effects is not clarified, one possibility is that 2B4 can be coupled to different types of intracellular signals that dictate whether 2B4 is stimulatory or inhibitory.

Through sites of tyrosine phosphorylation in its cytoplasmic domain, 2B4 associates with SAP, an intracellular adaptor molecule composed only of an SH2 domain and a short carboxyl-terminal tail of undetermined function^{7,14,15}. SAP is expressed in NK cells, T-cells, NK-T-cells and some B-cells, and is mutated in X-linked lymphoproliferative (XLP) disease in humans. This inherited disease is characterized by extreme vulnerability to Epstein-Barr virus (EBV) infection. Studies of NK cells from XLP patients demonstrated that SAP is necessary for the ability of 2B4 to trigger NK cell-mediated killing and IFN-γ production¹⁶⁻¹⁹. This function correlates with the capacity of SAP to bind the Src-related protein tyrosine kinase (PTK) FynT, via a second binding surface located around arginine 78 in the SAP SH2 domain²⁰. On the basis of these findings, it is believed that the stimulatory function of 2B4 is dependent on coupling of 2B4 to SAP-FynT-dependent protein tyrosine phosphorylation signals.

EAT-2 is a second member of the SAP family^{21,22}. Although little is known about this molecule, EAT-2 was found to interact in a manner analogous to SAP with SLAM-related receptors including 2B4, in overexpression systems such as yeast or Cos-1 cells²². However, EAT-2 does not share the binding site for FynT found in SAP. Consequently, it may possess an as yet unidentified signalling mechanism linking SLAM-related receptors to downstream signals.

Because of their non specific cytotoxic properties for antigen and their efficacy, NK cells constitute a particularly important population of effector cells in the development of immunoadoptive approaches for the treatment of cancer or infectious diseases. Generally, the current techniques for activating NK cells are based on using cytokines, usually in high doses which are often not well tolerated by the host. They involve culturing NK cells in the presence of different cytokines (such as IL-1, IL-2, IL-12, IL-15, IFNα, IFNγ, IL-6 and IL-4) used alone or in combination, which activation can be considerably increased by adhesion factors or co-stimulation factors such as ICAM, LFA or CD70. Similarly, in vivo, the efficacy of NK cells in anti-tumoral immunity is often not dissociable from co-administration of cytokines such as IL-2/IL-15 or IL-12, IL-18 and IL-10. Thus, most of the activation methods described in the prior art depend on the use of cytokines. Such methods have certain disadvantages linked to the toxic nature of many cytokines at high doses which cannot be used in in vivo applications, or to the non specific nature of many cytokines, the in vivo use of which risks being accompanied by undesirable side effects.

Therefore, there remains a real need for novel methods of activating NK cells.

There also remains a need for identifying new therapeutic targets allowing the modulation of NK cells, dendritic cells and macrophages functions.

In addition, there remains a need to develop new therapeutic strategies for the treatment of diseases or conditions such as infectious diseases, autoimmune diseases, and cancer.

The present invention seeks to meet these needs and other needs.

The present description refers to a number of documents, the content of which is herein incorporated by reference in their entirety.

### SUMMARY OF THE INVENTION

EAT-2 is a small intracellular molecule that is related to SAP and is expressed in innate immune cells including NK cells, DCs and macrophages. By way of one of its regions named SH2 (for Src homology 2) domain, EAT-2 binds to cell surface receptors termed SLAM family receptors. These receptors are implicated in modulating immune cell functions.

In order to learn more about the role of EAT-2, as well as of a closely related adaptor termed ERT, in NK cells, their function and mechanism of action were closely examined.

The present invention is based on the demonstration of the importance of EAT-2 and ERT in NK cells-mediated immune response and their identification as new therapeutic targets for the modulation of innate immune cells functions, more particularly, NK-cells functions. As shown herein, it was discovered that EAT-2 is a potent inhibitor of several activating pathways in NK cells. This has been demonstrated by the generation and characterization of EAT-2 deficient and overexpressing mice which show opposing phenotypes related to NK cells immune functions. Removal of EAT-2 from mouse NK cells was shown to generate NK cells with a greater capacity to kill tumour cells. Conversely, augmented expression of EAT-2 in mouse or human NK cells was found to suppress the capacity of NK cells to kill other cells. Further experiments revealed that the ability of EAT-2 to inhibit NK functions is dependent on tyrosine residues, which are located in a region after the SH2 domain of EAT-2 referred to as the “tail”. Mouse EAT-2 has two tyrosines in this region, whereas human EAT-2 has only one. These tyrosines become phosphorylated, and this phosphorylation enables EAT-2 to interact with other intracellular molecules referred to as “effectors” that are presumed to mediate its inhibitory effect.

Thus, EAT-2 and ERT were identified as novel therapeutic targets that modulate the functions of innate immune cells and more particularly of NK cells. These targets are especially useful in the treatment of cancer, infectious diseases and auto-immunity.

Thus, in one aspect, the present invention relates to the inhibition of the expression or functions of EAT-2 in order to enhance NK cell-mediated immune response (e.g., enhanced NK cells' ability to kill target cells) and to treat cancer and infectious (e.g., viral) illnesses or other diseases or conditions in which NK-cell-mediated immune response is desired.

In another aspect, the present invention relates to the inhibition of the expression or functions of EAT-2 in order to augment the efficacy of DCs in vaccination therapies against cancer and viral infections, or other diseases or conditions in which a an increase in NK cell-mediated immune response is desired.

In a further aspect, the present invention concerns the stimulation and augmentation of EAT-2 expression and activity to suppress or decrease innate immune cells (e.g., NK cells, macrophages and dendritic cells) function useful for the treatment of autoimmune diseases such as lupus, rheumatoid arthritis and others.

Thus, the present invention generally features novel methods of modulating innate immune cells-mediated immune response for the treatment of various diseases such as infectious diseases, cancers and autoimmune diseases.

In one embodiment, the methods of the present invention comprise a modulation of the expression of EAT-2 in a cell or organism. Such methods include, in particular embodiments, the use of an antisense nucleic acid of EAT-2, of EAT-2 siRNAs or of an EAT-2 specific ribozyme. Other agents, which decrease the expression level and/or activity of EAT-2 (e.g., antibodies (vaccines), small molecules, peptides) are also encompassed as agents useful for enhancing NK cells functions (e.g., NK cells' ability to kill target cells) and to treat infectious illnesses and cancers.

Thus, in a related aspect, the present invention concerns antisense oligonucleotides hybridizing to a nucleic acid sequence encoding EAT-2 protein (FIG. 15) thereby enabling the control of the transcription or translation of the EAT-2 gene in cells. The antisense sequences of the present invention consist of all or part of the EAT-2 nucleic acid sequence (FIG. 15, Genbank Accession number NM_{—}053282) in reverse orientation, and variants thereof. The present invention further relates to small double stranded RNA molecules (siRNAs) derived from EAT-2 nucleic acid sequence (FIGS. 13A and B, 15 and variants thereof) which also decrease EAT-2 protein cell expression. In a particular embodiment, the present invention relates to antisense oligonucleotides and siRNAs that inhibit the expression of a specific EAT-2 splice variant. The present invention also relates to methods utilizing siRNA or antisense RNA to reduce EAT-2 mRNA and/or protein expression and therefore, to increase NK cells functions which are dependent on EAT-2 expression and biological activity. In a particular embodiment, inhibition or reduction of EAT-2 expression significantly increases the ability of NK cells to kill target cells. In another embodiment, inhibition or reduction of EAT-2 expression significantly increases the production of IFN-γ by NK cells. The EAT-2 complementary sequences of the present invention can either be directly transcribed in target cells or synthetically produced and incorporated into cells by well-known methods.

In a related aspect, the present invention features a method of reducing EAT-2 expression in a subject by administering thereto a RNA, or derivative thereof (e.g., siRNA, antisense RNA, etc), or vector producing same in an effective amount, to reduce EAT-2 expression, thereby increasing NK cells functions and treating or preventing diseases such as infectious diseases and cancer. The RNA (e.g., siRNA, antisense RNA, etc) can be modified so as to be less susceptible to enzymatic degradation or to facilitate its delivery to a target cell (e.g., NK cell, dendritic cell, macrophage etc.). RNA interference (i.e., RNAi) toward a targeted DNA segment in a cell can be achieved by administering a double stranded RNA (e.g., siRNA) molecule to the cell, wherein the ribonucleotide sequence of the double stranded RNA molecule corresponds to the ribonucleotide sequence of the targeted DNA segment. In one particular case where the siRNA or antisense RNA is chemically modified or contains point mutations, the antisense region of the siRNAs or antisense RNA, of the present invention is still capable (i.e. of maintaining its ability to hybridize to the target sequence) of hybridizing to the ribonucleotide sequence of the targeted gene (e.g., EAT-2 mRNA) and to inhibit its expression (e.g., trigger RNAi).

In another embodiment, the present invention relates to the use of EAT-2 specific ribozymes to reduce EAT-2 expression in cells and thus to increase NK cell functions (e.g., increase ability to kill target cells, increase IFN-γ production, etc). As well known in the art, ribozymes are enzymatic nucleic acid molecules capable of catalyzing the cleavage of other separate nucleic acid molecules in a nucleotide base sequence-specific manner. They can be used to target virtually any RNA transcript (see for example U.S. Pat. No. 6,656,731). Such event renders the targeted mRNA non-functional and abrogates protein expression of the target RNA. Thus, in accordance with one embodiment of the present invention EAT-2 expression is inhibited by the use of EAT-2 specific ribozymes in order to enhance innate immune cells (e.g., NK cells, macrophage and dendritic cells) function.

In another embodiment, the present invention relates to methods of inhibiting EAT-2 biological activity in cells. Thus, the present invention concerns the use of EAT-2 inhibitors to increase innate immune cells-mediated immune response, including the ability of NK cells to kill target cells (e.g., cancer cells, virus-infected cells, etc) and therefore to treat infectious diseases, cancers and other related illnesses. Without being limited to a particular mechanism of action, EAT-2 inhibitors of the present invention increase the ability of NK cells to kill target cells and increase IFN-γ production by NK cells.

In one aspect, the inhibitors of the present invention reduce or completely abolish EAT-2 biological activity. In a particular embodiment, the inhibitors of the present invention compete with natural endogenous EAT-2 interacting molecules (e.g., SLAM related receptors such as 2B4, effector proteins, phosphatases, etc) for binding to EAT-2. This reduces the inhibitory activity of EAT-2 toward NK cells function and thus increases NK cells ability to mediate an appropriate immune response. For example, peptides or small molecules mimicking EAT-2 interacting domains (e.g., EAT SH2 domain or EAT c-terminal “tail” domain) responsible for the coupling of EAT-2 to downstream effectors, etc) can be used to inhibit EAT-2 interaction with endogenous proteins. Alternatively, peptides or small molecules mimicking EAT-2 interacting proteins (or interacting domain thereof) can also be used to compete with endogenous proteins for the binding to EAT-2.

In a related embodiment, the compounds of the present invention specifically inhibit phosphorylation or promote dephosphorylation of EAT-2, thereby increasing innate immune cells function (e.g., the ability of NK cells to kill target cells, the production of IFN-γ by NK cells, etc).

Because EAT-2 deficient animals have enhanced NK cells functions, the use of a molecule (i.e. antibody, antibody fragment) directed against EAT-2 to increase NK cell-dependent immune response is an alternative and/or complementary way to increase the ability of NK cells to mediate an appropriate immune response (e.g., by killing target cells). For example, the enhancement of NK cells functions can be achieved by using specific EAT-2 antibodies targeting the SH2 domain of EAT-2 that interacts with SLAM related receptors or the tail domain that is phosphorylated and interacts with effector proteins. In the specific case of an EAT-2 vaccine, the EAT-2 exogenous sequence may be linked to other molecules including diphtheria toxin, other immunogenic toxin peptides or helper antigen peptides in order to improve its efficiency in eliciting the desired immunological response in vivo. Humanized mouse monoclonal antibodies or DNA vaccines comprising an EAT-2 nucleic acid sequence or fragment thereof (for an example of DNA vaccines see U.S. Pat. No. 6,472,375) may be used in accordance with the present invention to stimulate and increase an NK cell-mediated immune response and thus to prevent or treat viral infections, cancers or other diseases or conditions.

In another aspect, the present invention features novel methods of decreasing the function of innate immune cells (e.g., NK cells, dendritic cells and macrophages) to treat a disease or condition associated with innate immune cells function, such as autoimmune diseases including lupus, rheumatoid arthritis and others. In one embodiment, inhibition of an immune response mediated by NK-cells or other EAT-2 expressing cells (e.g., dendritic cells and macrophage) is achieved by stimulating the expression of EAT-2. In another embodiment, the inhibition an immune response mediated by NK-cells or other EAT-2 expressing cells is accomplished by gene delivery of EAT-2 using conventional vectors for gene therapy such as adenoviral vectors, retroviral vectors or any other suitable vector, as well known in the art.

The innate immune cells (NK cells, dendritic cells and macrophages) activation and inhibition methods of the present invention can be carried out in vitro, ex vivo and in vivo.

In a further embodiment, the present invention relates to screening assays to identify compounds that modulate the biological activity of EAT-2 (and/or ERT).

In one particular aspect, the present invention relates to screening assays to identify compounds (e.g., peptides, nucleic acids, small molecules) that completely or partially inhibit the expression of EAT-2 (and or ERT), thereby increasing innate immune cells functions.

In another aspect, the invention provides assays for screening candidates or test compounds, which bind to or modulate the activity of an EAT-2 protein or polypeptide or biologically active portion thereof. Thus, screening assays to identify compounds which stimulate or reduce EAT-2 expression or activity are encompassed by the present invention. Such compounds may be useful in the treatment of cancers, infectious diseases and autoimmune diseases such as lupus and rheumatoid arthritis.

In one embodiment, the assay is a cell-based assay in which a cell which expresses a EAT-2 protein or biologically active portion thereof, either natural or of recombinant origin, is contacted with a test compound and the ability of same to modulate a biological activity of EAT-2, e.g., interaction with SLAM related receptors, tyrosine phosphorylation of EAT-2, interaction with downstream effectors, production of IFN-γ, ability of EAT-2 expressing NK-cells to kill target cells or any other measurable biological activity of EAT-2, is determined. Determining the ability of same to modulate EAT-2 activity can also be accomplished by monitoring, for example, the expression and/or activity of a specific gene modulated by a EAT-2-dependent signalization cascade in the presence of the test compound as compared to the expression and/or activity in the absence thereof.

In yet a further embodiment, modulators of EAT-2 expression are identified in a method wherein a cell is contacted with a candidate compound and the expression of EAT-2 mRNA or protein in the cell is determined. The level of expression of EAT-2 mRNA or protein in the presence of the candidate compound is compared to the level of expression of EAT-2 mRNA or protein in the absence of the candidate compound. The candidate compound can then be identified as a modulator of EAT-2 expression based on this comparison. For example, when expression of EAT-2 mRNA or protein is greater (statistically significantly greater) in the presence of the candidate compound than in its absence, the candidate compound is identified as a stimulator of EAT-2 mRNA or protein expression. Alternatively, when expression of EAT-2 mRNA or protein is less (statistically significantly less) in the presence of the candidate compound than in its absence, the candidate compound is identified as an inhibitor of EAT-2 mRNA or protein expression. The level of EAT-2 mRNA or protein expression in the cells can be determined by methods described herein or other methods known in the art for detecting EAT-2 mRNA or protein.

In one embodiment, the screening assays of the present invention comprise: 1) contacting an EAT-2 protein, or functional variant thereof, with a candidate compound; and 2) measuring a biological activity of EAT-2, or variant thereof, in the presence of the candidate compound, wherein a compound that inhibits EAT-2 function is selected when a EAT-2 biological activity is significantly reduced in the presence of said candidate compound as compared to in the absence thereof.

The compounds identified by the screening assays of the present invention can be used as competitive or non-competitive inhibitors in assays to screen for, or to characterize similar or new EAT-2 antagonists. In competitive assays, the compounds of the present invention can be used without modification or they can be labelled (i.e., covalently or non-covalently linked to a moiety which directly or indirectly provide a detectable signal). Examples of labels include radiolabels such as 125I, 14C, and 3H, enzymes such as alkaline phosphatase and horseradish peroxidase (U.S. Pat. No. 3,645,090), ligands such as biotin, avidin, and luminescent compounds including bioluminescent, phosphorescent, chemiluminescent and fluorescent labels (U.S. Pat. No. 3,940,475).

In a related aspect, the present invention also relates to the use of any compound capable of inhibiting (antagonist, e.g., compound which reduces the phosphorylation of EAT-2) or stimulating (agonist, e.g., compound which stimulates the phosphorylation of EAT-2) EAT-2 expression in a cell for the preparation of a pharmaceutical composition intended for the enhancement or stimulation of NK cells-mediated immune response including for example the treatment or prevention of infectious diseases and cancers.

In a further embodiment, the present invention features pharmaceutical composition comprising a compound of the present invention (e.g., antisense, siRNA, ribozyme, peptides, nucleic acids, small molecules, antibodies etc) which can be chemically modified, in a pharmaceutically acceptable carrier or diluent. In another embodiment, the present invention features a method for treating or preventing a disease or condition in a subject (e.g., viral infections, cancers, autoimmune diseases), comprising administering to the subject a composition of the invention under conditions suitable for the treatment or prevention of the disease or condition in the subject (e.g., viral infections, cancers, autoimmune diseases), alone, or in conjunction with one or more therapeutic compounds.

In one embodiment, pharmaceutical compositions of the present invention comprise a specific nucleic acid sequence (e.g., a mammalian EAT-2 sequence, siRNA, antisense and the like) or fragment thereof in a vector, under the control of appropriate regulatory sequences to target its expression into a cell (e.g., NK cells, dendritic cells or macrophages) thereby increasing or decreasing NK cells and/or other innate immune cells functions.

The methods of the present invention can be used for subjects with pre-existing condition (e.g., already suffering from a viral infection or having a cancer), or subject predisposed to such condition. Thus, the present invention also relates to a prevention or prophylaxy of a disease or condition using the reagents and methods of the present invention.

The compounds of the present invention include lead compounds and derivative compounds constructed so as to have the same or similar molecular structure or shape, as the lead compounds, but may differ from the lead compounds either with respect to susceptibility to hydrolysis or proteolysis (e.g., bioavailability), or with respect to their biological properties (e.g., increased affinity for EAT-2). The present invention also relates to compounds and compositions that are useful for the treatment or prevention of conditions, diseases or disorders associated with inappropriate EAT-2 production or function.

In another embodiment, the present invention also relates to pharmaceutical compositions comprising one or more of the compounds described herein and a physiologically acceptable carrier. These pharmaceutical compositions can be in a variety of forms including oral dosage forms, topic creams, suppository, nasal spray and inhaler, as well as injectable and infusible solutions. Methods for preparing pharmaceutical composition are well known in the art as reference can be made to Remington's Pharmaceutical Sciences, Mack Publishing Company, Eaton, Pa., USA.

The compounds of the present invention can be administered to a subject to completely or partially inhibit the activity of EAT-2 in vivo. Thus the methods of the present invention are useful in the therapeutic treatment of EAT-2 related diseases which would benefit from an enhanced NK cell-mediated immune response (e.g., infectious diseases and cancers). For example, the compositions of the present invention can be administered in a therapeutically effective amount to treat symptoms related to inappropriate or insufficient activity of NK cells. In addition, the compounds of the present invention may be utilized alone or in combination with any other appropriate therapies (e.g., anti-viral, anti-cancer, anti-inflammatory therapies, etc), as determined by the practitioner.

In another aspect, the present invention features cells (e.g., NK cells) overexpressing EAT-2 and/or ERT useful to screen for agents that enhance NK cells functions (e.g., production of IFN-γ, ability to kill target cells etc).

The present invention also concerns transgenic mice, bearing at least one copy of a highly expressed EAT-2 gene or ERT gene, and their use as an animal model for diseases involving reduced NK cell-mediated immune response (e.g., viral infections, cancers). Such an animal model is also useful for assessing the ability of potential therapeutic compounds to stimulate NK-cells mediated immune response.

The present invention also concerns EAT-2 and ERT deficient mice (knock out mice), expressing less (as compared to normal, control mice) or no EAT-2 and/or ERT protein, and their use as an animal model for diseases involving increased innate immune cells (e.g., NK cells, dendritic cells, macrophages)-mediated immune response.

In addition, the present invention relates to methods of obtaining a genetically modified non-human animal having a deficiency or an increase in EAT-2 and/or ERT proteins, and their use as animal models for diseases involving reduced or increased innate immune cell-mediated immune response (e.g., viral infections, cancers, autoimmune diseases, etc).

In a further aspect, the present invention concerns EAT-2, ERT, CRACC and CD84 antibodies. In one particular embodiment, the present invention concerns polyclonal antibodies, monoclonal antibodies and humanized antibodies against EAT-2 and ERT. More specifically, the EAT-2 antibodies recognize the SH2 or tail domain of EAT-e or ERT. In another embodiment, the antibodies of the present invention are specific for phosphorylated EAT-2 or ERT proteins.

In another embodiment, the present invention concerns the use of EAT-2 or ERT deficient animals to produce an array of EAT-2 or ERT specific antibodies, notably monoclonal antibodies.

In order to provide a clear and consistent understanding of terms used in the specification and claims, including the scope to be given such terms, a number of definitions are provided herein below.

### DEFINITIONS

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), Rieger et al., Glossary of genetics: Classical and molecular, 5th edition, Springer-Verlag, New-York, 1991; Alberts et al., Molecular Biology of the Cell, 4th edition, Garland science, New-York, 2002; and, Lewin, Genes VII, Oxford University Press, New-York, 2000. Generally, the methods traditionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extraction of proteins, ethanol or isopropanol precipitation of DNA in saline medium, transformation into bacteria or transfection into cells, procedure for cell culture, infection, methods and the like are common methods used in the art. Such standard techniques can be found in reference manuals such as for example Sambrook et al. (2000, Molecular Cloning—A Laboratory Manual, Third Edition, Cold Spring Harbour Laboratories); and Ausubel et al. (1994, Current Protocols in Molecular Biology, John Wiley & Sons, New-York). In addition, methods and procedures to produce transgenic animals are well-known in the art and described in details for example in: Hogan et al., 1994, Manipulating the Mouse Embryo, Cold Spring Harbor Laboratory Press; Nagy et al., 2002, Manipulating the Mouse Embryo, 3rd edition, Cold Spring Harbor Laboratory Press.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term about.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The term “activation” of innate immune cells such as NK cells, dendritic cells and macrophages within the context of the present invention designates an increase in any immune function of the particular type of cell (e.g., NK cells, macrophages and dendritic cells). For example, the “activation” of NK cells corresponds to an increase in the production of IFNγ and/or the cytotoxic activity of NK cells. These two parameters as well as other parameters can easily be measured using techniques which are known to the skilled person and illustrated in the examples. The activation of innate immune cells (e.g., NK cells) in the context of the present invention may be dependent or independent of the use of conventional cytokines. The term “activated” NK cells or innate immune cells as used within the context of the present invention designates cells with at least one of the properties mentioned above.

Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the terms “nucleic acid” and “polynucleotides” as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

The terminology “EAT-2 nucleic acid” or “EAT-2 polynucleotide” refers to a native EAT-2 nucleic acid sequence. Similarly, the terminology “ERT nucleic acid” or “ERT polynucleotide” refers to a native ERT nucleic acid sequence. In one embodiment, the human EAT-2 nucleic acid sequence is as set forth in FIGS. 15 and 16 respectively. In another embodiment, the mouse EAT-2 and ERT nucleic acid sequences encode EAT-2 or ERT protein sequence as set forth in FIGS. 1 and 13 respectively. In another particular embodiment, the EAT-2 or ERT nucleic acid encodes a splice variant or allelic variant of the EAT-2 or ERT gene (FIG. 16). In yet another embodiment, the EAT-2 or ERT nucleic acid encodes a functional derivative or fragment of the EAT-2 or ERT protein.

An “isolated nucleic acid molecule”, as is generally understood and used herein, refers to a polymer of nucleotides, and includes but should not be limited to DNA and RNA. The “isolated” nucleic acid molecule is purified from its natural in vivo state.

By “RNA” or “mRNA” is meant a molecule comprising at least one ribonucleotide residue. By ribonucleotide is meant a nucleotide with a hydroxyl group at the 2′ position of a 1′-D-ribo-furanose moiety. The term include double stranded RNA, single stranded RNA, isolated RNA such as partially purified RNA, essentially purified RNA, synthetic RNA, recombinantly produced RNA, as well as altered RNA that differs from naturally occurring RNA by the addition, deletion, substitution and/or alteration of one or more nucleotide. Such alterations can include addition of non-nucleotide material, such as to the end(s) of a siRNA or internally, for example at one or more nucleotides of the RNA molecule. Nucleotides in the RNA molecules of the instant invention can also comprise non-standard nucleotides or chemically synthesized nucleotides or deoxynucleotides. These altered RNAs can be referred to as analogs or analogs of naturally occurring RNA.

Complementary DNA (cDNA). Recombinant nucleic acid molecules synthesized by reverse transcription of messenger RNA (“mRNA”).

Expression. By the term “expression” is meant the process by which a gene or otherwise nucleic acid sequence produces a polypeptide. It involves transcription of the gene into mRNA, and the translation of such mRNA into polypeptide(s).

The term “vector” is commonly known in the art and defines a plasmid DNA, phage DNA, viral DNA and the like, which can serve as a DNA vehicle into which nucleic acid of the present invention can be cloned. Numerous types of vectors exist and are well known in the art. One specific type of vector is called a targeting vector which may be used for homologous recombination with an endogenous target gene in a cell. Homologous recombination occurs between two sequences (i.e. the targeting vector and endogenous gene sequences) that are partially or fully complementary. Homologous recombination may be used to alter a gene sequence in a cell (e.g., embryonic stem cells, (ES cells)) in order to completely shut down protein expression or to introduce point mutations, substitutions or deletions in the target gene sequence. Such method is used for example to generate transgenic animals and is well known in the art.

Expression Vector. A vector or vehicle similar to a cloning vector but which is capable of expressing a gene which has been cloned into it, after transformation into a host. The cloned gene (or nucleic acid sequence) is usually placed under the control of (i.e., operably linked to) certain control sequences such as promoter sequences which may be cell or tissue specific (e.g., innate immune cells).

Expression control sequences will vary depending on whether the vector is designed to express the operably linked gene (or nucleic acid sequence) in a prokaryotic and/or eukaryotic host and can additionally contain transcriptional elements such as enhancer elements, termination sequences, tissue-specificity elements, and/or translational initiation and termination sites. Vectors which can be used both in prokaryotic and eukaryotic cells are often called shuttle vectors. In particular embodiment, the control sequences may allow general expression (i.e. expression in a large number of cell types) or tissue specific or cell specific expression of a particular nucleic acid sequence (e.g., in innate immune cells).

A DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine Dalgarno sequences in addition to the −10 and −35 consensus sequences.

As used herein, the term “gene therapy” relates to the introduction and expression in an animal (preferably a human) of an exogenous sequence (e.g., a EAT-2 or ERT gene or cDNA sequence, a EAT-2 or ERT siRNA or antisense nucleic acid) to supplement, replace or inhibit a target gene (i.e., EAT-2 or ERT gene), or to enable target cells to produce a protein (e.g., a EAT-2 chimeric protein to target a specific molecule innate immune cells) having a prophylactic or therapeutic effect toward autoimmune diseases and other EAT-2 related diseases. In a particular embodiment, the exogenous sequence is of the same origin as that of the animal (human sequence). In another embodiment, the exogenous sequence is of a different origin (e.g., human exogenous sequence in mice (e.g., knock-in).

Nucleic acid sequences may be detected by using hybridization with a complementary sequence (e.g., oligonucleotide probes—see U.S. Pat. Nos. 5,503,980 (Cantor); 5,202,231 (Drmanac et al.); 5,149,625 (Church et al.); 5,112,736 (Caldwell et al.); 5,068,176 (Vijg et al.); and 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ by one nucleotide (see U.S. Pat. Nos. 5,837,832 and 5,861,242 (Chee et al.). In addition, any other well-known hybridization technique (Northern blot, dot blot, Southern blot) may be used in accordance with the present invention.

Nucleic Acid Hybridization. Nucleic acid hybridization depends on the principle that two single-stranded nucleic acid molecules that have complementary base sequences will reform the thermodynamically favoured double-stranded structure if they are mixed under the proper conditions. The double-stranded structure will be formed between two complementary single-stranded nucleic acids even if one is immobilized on a nitrocellulose filter. In the Southern or Northern hybridization procedures, the latter situation occurs. The DNA/RNA of the individual to be tested may be digested with a restriction endonuclease if applicable, prior to its fractionation by agarose gel electrophoresis, conversion to the single-stranded form, and transfer to nitrocellulose paper, making it available for reannealing to the hybridization probe. Non-limiting examples of hybridization conditions can be found in Ausubel, F. M. et al., Current protocols in Molecular Biology, John Wiley & Sons, Inc., New York, N.Y. (1994). For purposes of illustration, an example of moderately stringent conditions for testing the hybridization of a polynucleotide of the present invention with other polynucleotides includes prewashing in a solution of 5×SSC, 0.5% SDS, 1 mM EDTA (pH 8.0); hybridizing at 50° C.-60° C., 5×SSC and 100 μg/ml denatured salmon sperm DNA overnight (12-16 hours); followed by washing twice at 60° C. for 15 minutes with each of 2×SSC, 0.5×SSC and 0.2×SSC containing 0.1% SDS. For example for highly stringent hybridization conditions, the hybridization temperature is changed to 62, 63, 64, 65, 66, 67 or 68° C. One skilled in the art will understand that the stringency of hybridization can be readily manipulated, such as by altering the salt and SDS concentration of the hybridizing and washing solutions and/or temperature at which the hybridization is performed. The temperature and salt concentration selected is determined based on the melting temperature (Tm) of the DNA hybrid. Other protocols or commercially available hybridization kits using different annealing and washing solutions can also be used as well known in the art. The use of formamide in different mixtures to lower the melting temperature may also be used and is well known in the art.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.”

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) non standard base pairing (e.g., I:C) or may contain one or more residues (including a basic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. In reference to more specific nucleic acid molecules of the present invention, the binding free energy for a nucleic acid molecule with its complementary sequence is sufficient to allow the relevant function of the nucleic acid to proceed (e.g., RNAi activity). For example, the degree of complementarity between the sense and antisense region (or strand) of the siRNA construct can be the same or can be different from the degree of complementarity between the antisense region of the siRNA and the target RNA sequence (e.g., EAT-2 or ERT RNA sequence). Complementarity to the target sequence of less than 100% in the antisense strand of the siRNA duplex (including deletions, insertions and point mutations) is reported to be tolerated when these differences are located between the 5′-end and the middle of the antisense siRNA (Elbashir et al., 2001, EMBO, 20(23):68-77-6888). Determination of binding free energies for nucleic acid molecules is well known in the art (e.g., see Turner et al., 1987, J. Am. Chem. Soc. 190:3783-3785; Frier et al., 1986 Proc. Nat. Acad. Sci. USA, 83: 9373-9377) “Perfectly complementary” means that all the contiguous residues of a nucleic acid molecule will hydrogen bond with the same number of contiguous residues in a second nucleic acid sequence. Appropriate hybridization conditions are well known to those skilled in the art, can be predicted readily based on sequence composition and conditions, or can be determined empirically by using routine testing (see Sambrook et al., (cf. Molecular Cloning: A Laboratory Manual, Third Edition, edited by Cold Spring Harbor Laboratory, 2000) at §§1.90-1.91, 7.37-7.57, 9.47-9.51 and 11.47-11.57, particularly at §§9.50-9.51, 11.12-11.13, 11.45-11.47 and 11.55-11.57). Sequences that are “sufficiently complementary” allow stable hybridization of a probe sequence to a target sequence, even if the two sequences are not completely identical.

A detection step may use any of a variety of known methods to detect the presence of nucleic acid by hybridization to a probe oligonucleotide. One specific example of a detection step uses a homogeneous detection method such as described in detail previously in Arnold et al. Clinical Chemistry 35:1588-1594 (1989), and U.S. Pat. Nos. 5,658,737 (Nelson et al.), and 5,118,801 and 5,312,728 (Lizardi et al.).

The types of detection methods in which probes can be used include Southern blots (DNA detection), dot or slot blots (DNA, RNA), and Northern blots (RNA detection). Labelled proteins could also be used to detect a particular nucleic acid sequence to which it binds (e.g., protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g., an array) different probes.

A “label” refers to a molecular moiety or compound that can be detected or can lead to a detectable signal. A label is joined, directly or indirectly, to a nucleic acid probe or the nucleic acid to be detected (e.g., an amplified sequence). Direct labelling can occur through bonds or interactions that link the label to the nucleic acid (e.g., covalent bonds or non-covalent interactions), whereas indirect labelling can occur through the use of a “linker” or bridging moiety, such as additional oligonucleotide(s), which is/are either directly or indirectly labelled. Bridging moieties may amplify a detectable signal. Labels can include any detectable moiety (e.g., a radionuclide, ligand such as biotin or avidin, enzyme or enzyme substrate, reactive group, chromophore such as a dye or coloured particle, luminescent compound including a bioluminescent, phosphorescent or chemiluminescent compound, and fluorescent compound). In one particular embodiment, the label on a labelled probe is detectable in a homogeneous assay system, i.e., in a mixture, the bound label exhibits a detectable change compared to an unbound label.

Other methods of labelling nucleic acids are known whereby a label is attached to a nucleic acid strand as it is fragmented, which is useful for labelling nucleic acids to be detected by hybridization to an array of immobilized DNA probes (e.g., see PCT No. PCT/IB99/02073).

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural, rare or synthetic nucleotides. They can be designed to enhance a chosen criterion like stability, for example. Chimeras of deoxyribonucleotides and ribonucleotides may also be within the scope of the present invention.

“Amplification” refers to any known in vitro procedure for obtaining multiple copies (“amplicons”) of a target nucleic acid sequence or its complement or fragments thereof. In vitro amplification refers to the production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification, nucleic acid sequence-based amplification (NASBA), and strand-displacement amplification (SDA). Replicase-mediated amplification uses self-replicating RNA molecules, and a replicase such as Qβ-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification (TMA) can also be used in the present invention. In one embodiment, TMA and NASBA isothermic methods of nucleic acid amplification are used. Those skilled in the art will understand that the oligonucleotide primer sequences of the present invention may be readily used in any in vitro amplification method based on primer extension by a polymerase (see generally Kwoh et al., 1990, Am. Biotechnol. Lab. 8:14 25 and (Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., (cf. Molecular Cloning: A Laboratory Manual, Third Edition, edited by Cold Spring Harbor Laboratory, 2000). As commonly known in the art, the oligos are designed to bind to a complementary sequence under selected conditions.

As used herein, a “primer” defines an oligonucleotide which is capable of annealing to a target sequence, thereby creating a double stranded region which can serve as an initiation point for nucleic acid synthesis under suitable conditions. Primers can be, for example, designed to be specific for certain alleles so as to be used in an allele-specific amplification system. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Of course the design of a primer from a known nucleic acid sequence is well known in the art. As for the oligos, it can comprise a number of types of different nucleotides.

As used herein, the twenty natural amino acids and their abbreviations follow conventional usage. Stereoisomers (e.g., D-amino acids) such as a,a-disubstituted amino acids, N-alkyl amino acids, lactic acid and other unconventional amino acids may also be suitable components for the polypeptides of the present invention. Examples of unconventional amino acids include but are not limited to selenocysteine, citrulline, ornithine, norvaline, 4-(E)-butenyl-4(R)-methyl-N-methylthreonine (MeBmt), N-methyl-leucine (MeLeu), aminoisobutyric acid, statine, N-methyl-alanine (MeAla).

As used herein, “protein” or “polypeptide” means any peptide-linked chain of amino acids, regardless of post-translational modifications (e.g., acetylation, phosphorylation, glycosylation, sulfatation, sumoylation, prenylation, ubiquitination, etc). A “EAT-2 protein” or a “EAT-2 polypeptide” is an expression product of EAT-2 nucleic acid (e.g., EAT-2 gene) such as native human EAT-2 protein (FIG. 13), a EAT-2 natural splice variant, a EAT-2 allelic variant (e.g., FIG. 16) or a EAT-2 protein homolog (e.g., mouse EAT-2, FIG. 13) that shares at least 60% (but preferably, at least 65, 70, 75, 80, 85, 86, 87, 88, 89, 90, 91, 92, 93, 94, 95, 96, 97, 98, 99, 100%) amino acid sequence identity with EAT-2 and displays functional activity of native EAT-2 protein. For the sake of brevity, the units (e.g., 66, 67 . . . 81, 82% . . . ) have not been specifically recited but are nevertheless considered within the scope of the present invention.

A “EAT-2 interacting protein” refers to a protein which binds directly or indirectly (e.g., via RNA or another bridging protein or molecule) to EAT-2 in order to modulate or participate in a functional activity of EAT-2. These proteins include kinases, phosphatases, scaffolding proteins, effector proteins, SLAM related receptors (e.g., 2B4) or any other proteins known to interact with EAT-2 (see below). An “isolated protein” or “isolated polypeptide” is purified from its natural in vivo state.

The terms “biological activity” or “functional activity” or “function” are used interchangeably and refer to any detectable biological activity associated with a structural, biochemical or physiological activity of a cell or protein (i.e. EAT-2 or ERT protein, NK cells, macrophages, dendritic cells, etc). For instance, one non-limiting example of a functional activity of EAT-2 protein includes interacting with SLAM-related receptors (e.g., 2B4). The SH2 and “tail” domains are among the specific sequences that interact with EAT-2 interacting proteins. Other specific non-limiting examples of EAT-2 interacting proteins include kinases, phophatases and effector proteins. Therefore, interaction of EAT-2 with any of these EAT-2 interacting proteins is considered a functional activity of an EAT-2 protein. Thus, oligomerization of EAT-2 with specific proteins such as proteins containing SH2, domains as well as with itself is also considered a biological activity of EAT-2. Such interaction may be stable or transient. Another example of an EAT-2 functional activity is its capacity to become phosphorylated by several kinases. Thus, in accordance with the present invention, oligomerization and phosphorylation of EAT-2 are also considered as functional or biological activities of EAT-2. Interaction of EAT-2 with other known ligands (e.g., phophatases, effector proteins, etc) not explicitly listed in the present invention may also be considered functional activities of EAT-2. Thus, in accordance with the present invention, measuring the effect of a test compound on its ability to inhibit or increase (e.g., modulate) EAT-2 binding or interaction, level of expression as well as phosphorylation status is considered herein as measuring a biological activity of EAT-2.

As noted above, EAT-2 biological activity also includes any biochemical measurement of the protein, conformational changes, phosphorylation status (or any other posttranslational modification e.g., ubiquitination, sumolylation, palmytoylation, prenylation etc), any downstream effect of EAT-2's signalling such as protein phosphorylation in signalling cascades, indirect gene expression modulation, or any other feature of the protein that can be measured with techniques known in the art. Finally, EAT-2 biological activities include a detectable change in IFN-γ production, a change in the ability of NK cells to kill target cells (cytotoxicity) and any other cell phenotype that is modulated by the action of EAT-2. All of the above activities are also applicable to ERT.

EAT-2 or ERT antibody. As used herein, the term “EAT-2 antibody” or “immunologically specific EAT-2 antibody” refers to an antibody that specifically binds to (interacts with) a EAT-2 protein and displays no substantial binding to other naturally occurring proteins other than the ones sharing the same antigenic determinants as the EAT-2 protein (e.g., ERT). Similarly, the term “ERT antibody” or “immunologically specific ERT antibody” refers to an antibody that specifically binds to (interacts with) an ERT protein and displays no substantial binding to other naturally occurring proteins other than the ones sharing the same antigenic determinants as the EAT-2 protein. EAT-2 and ERT antibodies include polyclonal, monoclonal, humanized as well as chimeric antibodies.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

The term “animal” as used herein in the context of a transgenic or knock out animal includes all vertebrate animals except humans. It also includes an individual animal at all stages of development including embryonic and foetal stages. When used broadly in the context of treating diseases or conditions, EAT-2 or ERT sequences and the like, the term animal encompasses human.

A “transgenic animal” is any animal (except human) containing one or more cells bearing genetic information altered or received, directly or indirectly, by deliberate genetic manipulation at the subcellular level, such as targeted recombination (homologous recombination) or microinjection or infection with recombinant virus. The term transgenic animal is not meant to encompass classical cross-breading or in vitro fertilization but rather is meant to include animals in which one or more cells are altered by or received a recombinant DNA molecule. This molecule may be targeted to a specific genetic locus, be randomly integrated within a chromosome or it may be extrachromosomally replicating DNA.

The term “germ cell line transgenic animal” refers to a transgenic animal in which the genetic alteration or genetic information was introduced into a germ-line cell, thereby conferring the ability to transfer the genetic information to offspring. In the case where such offspring possess some or all of that alteration or genetic information, then they too are also considered transgenic animals.

As used herein, a “targeted gene” or “knock out” is a DNA sequence introduced into a germline or a non-human animal by way of human intervention, including, but not limited to, the methods described herein (e.g., homologous recombination, random integration . . . ). The targeted genes of the present invention include DNA sequences which are designed to alter cognate endogenous alleles (e.g., EAT-2 or ERT).

As used herein, the designation “functional derivative” denotes, in the context of a functional derivative of an amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The substituting amino acid generally has chemico-physical properties, which are similar to that of the substituted amino acid. The similar chemico-physical properties include, similarities in charge, bulkiness, hydrophobicity, hydrophilicity and the like. The term “functional derivatives” is intended to include “segments”, “variants”, “analogs” or “chemical derivatives” of the subject matter of the present invention.

As used herein, “chemical derivatives” is meant to cover additional chemical moieties not normally part of the subject matter of the invention. Such moieties could affect the physico chemical characteristic of the derivative (i.e. solubility, absorption, half life and the like, decrease of toxicity). Such moieties are exemplified in Remington: The Science and Practice of Pharmacy by Alfonso R. Gennaro, 2003, 21st edition, Mack Publishing Company. Methods of coupling these chemical physical moieties to a polypeptide are well known in the art.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutation of nucleic acid molecule is a mutant nucleic acid molecule. A mutant polypeptide can be encoded from this mutant nucleic acid molecule.

The term “variant” refers herein to a protein, which is substantially similar in structure and biological activity to the protein, or nucleic acid of the present invention to maintain at least one of its biological activities. Thus, provided that two molecules possess a common activity and can substitute for each other, they are considered variants as that term is used herein, even if the composition, or secondary, tertiary or quaternary structure of one molecule is not identical to that found in the other, or if the amino acid sequence or nucleotide sequence is not identical. A homolog is a gene sequence encoding a polypeptide isolated from an organism other than a human being. Similarly, a homolog of a native polypeptide is an expression product of a gene homolog. Expression vectors, regulatory sequences (e.g., promoters), leader sequences and method to generate same and introduce them in cells are well known in the art.

Amino acid sequence variants of the polypeptides of the present invention (e.g., EAT-2, ERT, SAP) can be prepared by mutations in the DNA. Such variants include, for example, deletions from, or insertions or substitutions of, residues within the amino acid sequence shown in SEQ ID NOs: 1-6. Any combination of deletion, insertion, and substitution can also be made to arrive at the final construct, provided that the final construct possesses the desired activity.

While the site for introducing an amino acid sequence variation is predetermined, the mutation per se need not be predetermined. For example, to optimize the performance of a mutation at a given site, random mutagenesis can be conducted at the target codon or region and the expressed polypeptide (e.g., EAT-2, ERT, SAP) variants screened for the optimal combination of desired activity. Techniques for making substitution mutations at predetermined sites in DNA having a known sequence are well known in the art and include, for example, site-specific mutagenesis.

Preparation of a variant in accordance with the present invention is preferably achieved by site-specific mutagenesis of DNA that encodes an earlier prepared variant or a nonvariant version of the protein. Site-specific mutagenesis allows the production of variants through the use of specific oligonucleotide sequences that encode the DNA sequence of the desired mutation. In general, the technique of site-specific mutagenesis is well known in the art, as exemplified by publications such as Adelman et al., DNA 2:183 (1983) and Ausubel et al. “Current Protocols in Molecular Biology”, J. Wiley & Sons, NY, N.Y., 1996.

Amino acid sequence deletions generally range from about 1 to 30 residues, more preferably 1 to 10 residues, and typically are contiguous.

Amino acid sequence insertions include amino and/or carboxyl-terminal fusions of from one residue to polypeptides of essentially unrestricted length, as well as intrasequence insertions of single or multiple amino acid residues. Intrasequence insertions (i.e., insertions within the complete EAT-2/ERT/SAP sequence) can range generally from about 1 to 10 residues, more preferably 1 to 5.

The third group of variants are those in which at least one amino acid residue in the EAT-2/ERT/SAP molecule, has been removed and a different residue inserted in its place. Such substitutions preferably are made in accordance with the following Table 1 when it is desired to modulate finely the characteristics of the polypeptide.

TABLE 1

| Original Residue | Exemplary Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | leu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Substantial changes in functional or immunological identity can be made by selecting substitutions that are less conservative than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine.

Some deletions and insertions, and substitutions are not expected to produce radical changes in the characteristics of the polypeptides of the present invention. However, when it is difficult to predict the exact effect of the substitution, deletion, or insertion in advance of doing so, one skilled in the art will appreciate that the effect will be evaluated by routine screening assays. For example, a variant typically is made by site-specific mutagenesis of the native EAT-2/ERT encoding-nucleic acid, expression of the variant nucleic acid in recombinant cell culture, and, optionally, purification from the cell culture, for example, by immunoaffinity adsorption on a column (to absorb the variant by binding it to at least one remaining immune epitope). The activity of the cell lysate or purified EAT-2/ERT molecule variant is then screened in a suitable screening assay for the desired characteristic. For example, a change in the immunological character of the polypeptide molecule, such as affinity for a given antibody, is measured by a competitive type immunoassay. Changes in immunomodulation activity are measured by the appropriate assay. Modifications of such protein properties as redox or thermal stability, hydrophobicity, susceptibility to proteolytic degradation or the tendency to aggregate with carriers or into multimers are assayed by methods well known to the ordinarily skilled artisan.

Binding agent. A binding agent is a molecule or compound that specifically binds to or interacts with an EAT-2 or ERT polypeptide. Non-limiting examples of binding agents include antibodies, interacting partners, ligands, and the like. It will be understood that such binding agents can be natural, recombinant or synthetic.

In accordance with the present invention, it shall be understood that the “in vivo” experimental model (e.g., a transgenic animal of the present invention) can also be used to carry out an “in vitro” assay. For example, cellular extracts from the indicator cells can be prepared and used in one of the aforementioned “in vitro” tests (such as in binding assays or in vitro translation assays).

The term “subject” or “patient” as used herein refers to an animal, preferably a mammal, and most preferably a human who is the object of treatment, observation or experiment.

As used herein, the term “purified” refers to a molecule (e.g., EAT-2 or ERT polypeptides, antisense or RNAi molecule, etc) having been separated from a component of the composition in which it was originally present. Thus, for example, a “purified EAT-2 polypeptide or polynucleotide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. Therefore, the terms “separating” or “purifying” refers to methods by which one or more components of the biological sample are removed from one or more other components of the sample. Sample components include nucleic acids in a generally aqueous solution that may include other components, such as proteins, carbohydrates, or lipids. A separating or purifying step preferably removes at least about 70% (e.g., 70, 75, 80, 85, 90, 95, 96, 97, 98, 99, 100%), more preferably at least about 90% (e.g., 90, 91, 92, 93, 94, 95, 96, 97, 98, 99, 100%) and, even more preferably, at least about 95% (e.g., 95, 96, 97, 98, 99, 100%) of the other components present in the sample from the desired component. For the sake of brevity, the units (e.g., 66, 67 . . . 81, 82, . . . 91, 92% . . . ) have not systematically been recited but are considered, nevertheless, within the scope of the present invention.

The terms “inhibiting,” “reducing” or any variation of these terms, when used in the claims and/or the specification includes any measurable decrease or complete inhibition of at least one biological activity of EAT-2 to achieve a desired result. For example, a compound is said to be inhibiting EAT-2 activity when an increase in the production of IFN-γ is measured following a treatment with the compounds of the present invention as compared to in the absence thereof. Other non-limiting examples include a reduction in the phosphorylation status of EAT-2 and in the ability of NK cells to kill target cells.

As used herein, the terms “molecule”, “compound”, “agent” or “ligand” are used interchangeably and broadly to refer to natural, synthetic or semi-synthetic molecules or compounds. The term “compound” therefore denotes for example chemicals, macromolecules, cell or tissue extracts (from plants or animals) and the like. Non-limiting examples of compounds include peptides, antibodies, carbohydrates, nucleic acid molecules and pharmaceutical agents. The compound can be selected and screened by a variety of means including random screening, rational selection and by rational design using for example protein or ligand (e.g., SLAM related receptor domains which interact with EAT-2 or ERT) modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, the modulating compounds of the present invention are modified to enhance their stability and their bioavailability. The compounds or molecules identified in accordance with the teachings of the present invention have a therapeutic value in diseases or conditions in which the physiology or homeostasis of the cell and/or tissue is compromised by EAT-2 production or response. For example, compounds of the present invention, by acting on a biological activity of EAT-2 (e.g., IFN-γ production) may increase the function/activity of innate immune cells such as NK cells and therefore treat cancers and/or infectious diseases. Alternatively, compounds of the present invention may reduce the function/activity of particular innate immune cells and are thus useful in the treatment of autoimmune diseases.

As used herein “antagonists”, “EAT-2 antagonists” or “EAT-2 inhibitors” refer to any molecule or compound capable of inhibiting (completely or partially) a biological activity of EAT-2. Similarly, “ERT antagonists” or “ERT inhibitors” refer to any molecule or compound capable of inhibiting (completely or partially) a biological activity of ERT. On the contrary, “agonists”, “EAT-2 agonists” or “EAT-2 stimulators” refer to any molecule or compound capable of enhancing or stimulating (completely or partially) a biological activity of EAT-2. The latter definition is also applicable to “ERT agonists” and “ERT stimulators”.

When referring to nucleic acid molecules, proteins or polypeptides, the term native refers to a naturally occurring nucleic acid or polypeptide. A homolog is a gene sequence encoding a polypeptide isolated from an organism other than a human being. Similarly, a homolog of a native polypeptide is an expression product of a gene homolog. Of course, the non-coding portion of a gene can also find a homolog portion in another organism.

As used herein, the term “pharmaceutically acceptable” refers to molecular entities and compositions that are physiologically tolerable and do not typically produce an allergic or similar untoward reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans. The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the expression of SAP family adaptors in normal mouse immune cells. a. Comparison of amino-acid sequences of mouse EAT-2, ERT and SAP. The residues that are strictly conserved in all three SAP-related adaptors are shown in the consensus sequence at the bottom. “+” indicates a similar, but not identical, residue. The positions of the SH2 domain and carboxyl-terminal tail are highlighted. “&” corresponds to SAP arginine 78, which is the epicentre of the binding site for the FynT SH3 domain. Asterisks highlight the two tyrosines in the tails of EAT-2 and ERT. b. Location of eat-2 and ert genes on mouse chromosome 1. The positions of the eat-2 and ert genes, as well as of the slam gene locus, on mouse chromosome 1 are depicted schematically. eat-2 and ert are separated from each other by approximately 26 KB, while they are separated from the slam locus by roughly 1300 KB. The transcriptional orientation of eat-2 and ert is indicated by an arrow. c. RT-PCRs. RT-PCRs were performed using RNA samples from the indicated cell populations. All PCRs were conducted under linear assay conditions.

FIG. 2 shows the Generation of eat-2^{-/-} and ert^{-/-} mice. a. Targeting strategy. A schematic representation of the mouse eat-2 and ert genes is shown at the top. The four coding exons are represented as boxes, whereas the positions of the initiating ATGs and the stop codons are shown as right-sided and left-sided arrows, respectively. The targeting vector used is presented in the middle. In essence, the neo gene was inserted in the antisense orientation in the middle of exon 2 of the genes. The targeted alleles are depicted at the bottom. b and c. Expression of SAP-related adaptors in targeted mice. The expression of SAP-related adaptors was determined by immunoblotting of equivalent amounts of total cell lysates from IL-2-activated NK cells, using the indicated antibodies. MAb 5A7 (top panels) is a MAb that recognizes EAT-2, but not ERT or SAP. MAb 15G3 (middle panels) is a MAb that reacts with EAT-2 and ERT, but not SAP. b. EAT-2-deficient mice. c. ERT-deficient mice. In the top panel of FIG. 2c, proteins were first immunoprecipitated with anti-EAT-2 MAb 5A7 and subsequently immunoblotted with MAb 15G3.

FIG. 3 shows the functional characterization of NK cells from eat-2^{-/-} and ert^{-/-} mice. a and b. Natural cytotoxicity. IL-2-activated NK cells were obtained from the indicated mice. They were then tested in in vitro cytotoxicity assays, using the specified targets previously labelled with ⁵¹Cr and at the indicated effector:target ratios (abscissa). Target cell lysis (ordinate) was measured after 4 hours and is expressed as percent of maximum release. All assays were done in duplicate. Ranges are shown as error bars. The following targets and their characteristics were utilized: RMA is a class I MHC(H-2^{b})-positive CD48-positive mouse T-cell lymphoma; RMA-S is an antigen processing (TAP)-deficient variant of RMA; YAC-1 is a class I MHC(H-2^{a})-positive CD48-positive mouse T-cell thymoma; YB2/0 (YB) is a class I MHC-positive rat myeloma cell line; CHO is a class I MHC-positive hamster ovary cell line. c and d. IFN-γ release. IL-2-activated NK cells were stimulated with the indicated antibodies (at 1.0 μg.ml⁻¹) coated on plastic. Anti-class I MHC H-2 D^{b} MAb KH95 was used as isotype control. Other controls included a combination of PMA and ionomycin (P+I), IL-12 or IL-18. After 20 hours, the release of IFN-γ in the supernatant was determined by ELISA. Assays were done in triplicates and were repeated at least three times. The ranges of the triplicate values are shown with error bars. Two different experiments are shown in FIG. 3c. e. Natural cytotoxicity in SAP-deficient NK cells. The impact of SAP deficiency on the ability of NK cells to kill targets was ascertained as detailed for FIGS. 3a and b.

FIG. 4 shows the functional analyses of NK cells from transgenic mice overexpressing EAT-2 or SAP. a. Overexpression of EAT-2 and SAP in transgenic NK cells. IL-2-activated NK cells were generated from transgenic mice expressing wild-type EAT-2 (EAT-2 wt) or SAP, and lysates were probed by immunoblotting with anti-EAT-2 (top panel) or anti-SAP (bottom panel). b. Natural killing. These assays were performed as outlined in the legend of FIGS. 3a and b. C4.4-25 is a class I MHC-negative CD48-positive variant of mouse T-cell thymoma cell line EL-4. c. IFN-γ release. These experiments were performed as described for FIGS. 3c and d, except that various concentrations (shown in brackets) of stimulatory antibodies were used.

FIG. 5 shows the association of EAT-2/ERT and SAP with 2B4, but not other SLAM-related receptors, in mouse NK cells. a. Association of phosphotyrosine-containing proteins with SAP-related adaptors in mouse NK cells. The indicated SAP-related adaptors were immunoprecipitated from IL-2-activated mouse NK cells, resolved in 8% gels and probed by immunoblotting with anti-phosphotyrosine (P.tyr). The anti-EAT-2 MAbs used for immunoprecipitation react with both EAT-2 and ERT. Anti-CD4 MAb GK1.5 was used as control antibody. IgH: heavy chain of IgG. Efficient immunoprecipitation of the SAP-related adaptors was verified by probing parallel immunoprecipitates with anti-EAT-2/ERT and anti-SAP (data not shown). b. Association of 2B4 with SAP-related adaptors in NK cells. Immunoprecipitates of SAP family adaptors were reprobed by immunoblotting with anti-2B4 (top panels), anti-CRACC (middle panels) and anti-CD84 (bottom panels). The migration of the various SLAM-related receptors was verified by immunoblotting of total cell lysates with the specified antibodies (lane 5). Longer autoradiographic exposures of these blots failed to show any evidence of association between SAP-related adaptors and CRACC or CD84 (data not shown).

FIG. 6 shows that EAT-2/ERT, but not SAP, are tyrosine phosphorylated in mouse NK cells. a. Tyrosine phosphorylation of EAT-2/ERT in normal mouse NK cells. The indicated SAP-related adaptors were immunoprecipitated from lysates of IL-2-activated mouse NK cells, and their phosphotyrosine content was assessed by immunoblotting with anti-P.tyr (top panel). Efficient immunoprecipitation of the SAP-related adaptors was verified by reprobing the immunoblot membrane with a mixture of anti-SAP and anti-EAT-2 (bottom panel). NRS: normal rabbit serum. b. Evidence that the carboxyl-terminal tyrosines of EAT-2 are phosphorylated. EAT-2 was immunoprecipitated from BI-141 cells expressing SLAM in the presence of the indicated EAT-2 polypeptides, and its phosphotyrosine content was ascertained by immunoblotting with anti-phosphotyrosine (P.tyr) (top panel). Levels of EAT-2 were controlled by reprobing with anti-EAT-2 (bottom panel).

FIG. 7 shows that the carboxyl-terminal tyrosines of EAT-2 are required for inhibition of NK cell functions. a. Overexpression of wild-type EAT-2 and EAT-2 Y2F in transgenic NK cells. IL-2-activated NK cells were generated from transgenic mice expressing wild-type EAT-2 (EAT-2 wt) or EAT-2 Y120,127F (“Y2F”), and lysates were probed by immunoblotting with anti-EAT-2 (top panel) or anti-SAP (bottom panel). b. Natural cytotoxicity. Assays were performed as outlined in the legend of FIGS. 3a and b. c. IFN-γ release. These experiments were performed as described for FIGS. 3c and d. d. The carboxyl-terminal tyrosines of EAT-2 are not required for association with 2B4. Cos-1 cells were transfected with cDNAs encoding wild-type EAT-2 or EAT-2 Y2F, in the presence of cDNAs coding for wild-type mouse 2B4 (long form) and ΔSH2FynT (which lacks the FynT SH2 domain, to avoid binding to EAT-2). After 48 hours, cells were lysed, and the ability of EAT-2 to associate with 2B4 was assessed by probing 2B4 immunoprecipitates with an anti-EAT-2 immunoblot (first panel). Tyrosine phosphorylation of EAT-2 was verified by reprobing the immunoblot membrane with anti-phosphotyrosine (P.tyr) (second panel), whereas the presence of 2B4 in the immunoprecipitates was confirmed by reprobing with anti-2B4 (bottom panel). Anti-Tac MAb 7G7 was used as control immunoprecipitating antibody.

FIG. 8 shows that EAT-2 inhibits proximal events in stimulatory NK receptor signalling. a. Enhanced CD16-induced protein tyrosine phosphorylation in EAT-2-deficient NK cells. IL-2-activated NK cells were stimulated or not for 1 minute with biotinylated anti-CD16 MAb 2.4G2 or anti-class I MHC MAb KH95, followed by avidin. Changes in protein tyrosine phosphorylation were determined by anti-phosphotyrosine (P.tyr) immunoblotting of total cell lysates (top panel). The abundance of 2B4 was assessed by reprobing with a rabbit anti-mouse 2B4 serum (bottom panel). b. Decreased CD16-mediated protein tyrosine phosphorylation in NK cells overexpressing wild-type EAT-2. This experiment was performed as outlined for FIG. 8a, except that NK cells from EAT-2 transgenic mice were used. Note that a slight (˜1.3-1.5 fold) decrease in the abundance and a retardation of the electrophoretic mobility of 2B4 (bottom panel) was observed in NK cells overexpressing wild-type EAT-2. The basis and significance of this finding is not known. c. The carboxyl-terminal tyrosines of EAT-2 are required for inhibition of NK signalling. This experiment was performed as detailed for FIG. 8a, except that NK cells were stimulated or not for 1 minute with biotinylated anti-2B4 MAb 2B4 or anti-class I MHC MAb KH95, followed by avidin.

FIG. 9 shows a model of NK inhibition by EAT-2. A possible mechanism by which EAT-2 inhibits the function of stimulatory NK receptors coupled to Src-related protein tyrosine kinases (Src) is proposed. In the case of mouse NKG2D, activation can occur through ITAM-dependent (DAP-12) or ITAM-independent (DAP-10) pathways; it is likely that both pathways require Src kinases.

FIG. 10 shows the detection of SAP-related proteins in mouse immune cells. a. Detection of SAP-related polypeptides in transfected cells. Lysates from BI-141 T-cells transfected with individual sap-related cDNAs were probed by immunoblotting with anti-EAT-2 (top panel) or anti-SAP (bottom panel). Note that the anti-EAT-2 antibodies used react both with EAT-2 and with ERT. b. Detection of SAP family adaptors in normal mouse immune cells. Lysates from the indicated cells were probed by immunoblotting with anti-EAT-2/ERT (top panel) and anti-SAP (bottom panel).

FIG. 11 shows the cell surface markers on freshly isolated NK cells from EAT-2-deficient mice. Red blood cell-depleted splenocytes were obtained from eat-2^{+/+} and eat-2^{-/-} mice and stained with the indicated antibodies. NK cells were identified by gating on DX5-positive CD3-negative cells. There was also no difference in the expression of cell surface markers between eat-2^{+/+} and eat-2^{-/-} NK cells when IL-2-activated NK cells were used for these studies (data not shown).

FIG. 12 shows the analyses of NK functions in ERT transgenic mice. a. Overexpression of ERT in transgenic NK cells. IL-2-activated NK cells were generated from transgenic mice overexpressing ERT, and lysates were probed by immunoblotting with anti-EAT-2/ERT (top panel) or anti-SAP (bottom panel). b. Natural cytotoxicity. Assays were performed as outlined in the legend of FIGS. 3a and b. c. IFN-γ release. These experiments were performed as described for FIGS. 3c and d.

FIG. 13. A) Shows the comparison of EAT-2 sequences from mouse (m), human (h) and chicken (c). The boundaries of the SH2 domain and tail are indicated. The locations of Y120 and Y127 are highlighted. B) Identical to FIG. 13A except that the human EAT-2 sequence comprises a lysine (K) at position 122 instead of any amino acid (N).

FIG. 14 shows the inhibition of NK cell functions by EAT-2 overexpression. Inhibition of natural killing by human EAT-2. Wild-type (wt) human (h) EAT-2 (A) or EAT-2 Y127F (B) was overexpressed in the human NK cell line YT-S, Natural killing towards K562 was measured using a ⁵¹Cr release assay. Note that a dominant-negative effect seems to be seen with hEAT-2 Y127F. E:T: effector-to-target cell ratio.

FIG. 15 shows a human EAT-2 cDNA sequence. The initiation (ATG) and stop codons are shown.

FIG. 16 shows a human EAT-2 genomic sequence. Location of the exons (highlighted), allelic variants (SNPs-highlighted and italic), as well as the initiation (boxed) and stop (underlined) codons are shown.

### DESCRIPTION OF ILLUSTRATIVE EMBODIMENTS

The present invention is based on the demonstration of the importance of EAT-2 and ERT in NK cells-mediated immune response and their identification as new therapeutic targets for the modulation of innate immune cells function, more particularly, NK-cells function.

It was discovered that EAT-2 is a potent inhibitor of several activating pathways in NK cells. EAT-2 is an adaptor expressed in innate immune cells including NK cells. Its relative, SAP, controls the function of SLAM-related receptors by recruiting FynT. The role of EAT-2 in NK cells was examined by creating mice that lack or overexpress EAT-2. Like SAP, EAT-2 is associated with SLAM-related receptor 2B4 in NK cells. However, unlike SAP, EAT-2 is an inhibitor of NK functions. It represses natural cytotoxicity and interferon-γ secretion, through a mechanism involving tyrosine phosphorylation of its tail. A similar function was demonstrated herein for ERT, a novel SAP family member expressed in mouse NK cells. The data presented herein therefore identify new therapeutic targets for the modulation of innate immune cells functions and unveil a new mechanism of NK inhibition.

### Therapeutic Nucleic Acids

The present invention has identified EAT-2 as a target for the treatment of modulation of innate immune cell activity such as NK cells useful in the treatment of infectious diseases, cancers and autoimmune diseases. Thus, in one embodiment, the present invention generally relates to EAT-2 expression modulation and the use of EAT-2 expression modulation (i.e. EAT-2 overexpression, and EAT-2 expression inhibition) to treat or prevent infectious diseases, cancers and autoimmune disease.

### SiRNAs

The present invention further concerns the use of RNA interference (RNAi) to decrease EAT-2 expression in target cells. “RNA interference” refers to the process of sequence specific suppression of gene expression mediated by small interfering RNA (siRNA) without generalized suppression of protein synthesis. While the invention is not limited to a particular mode of action, RNAi may involve degradation of messenger RNA (e.g., EAT-2 mRNA) by an RNA induced silencing complex (RISC), preventing translation of the transcribed targeted mRNA. Alternatively, it may involve methylation of genomic DNA, which shuts down transcription of a targeted gene. The suppression of gene expression caused by RNAi may be transient or it may be more stable, even permanent.

RNA interference is triggered by the presence of short interfering RNAs of about 20-25 nucleotides in length which comprise about 19 base pair duplexes. These siRNAs can be of synthetic origin or they can be derived from a ribonuclease III activity (e.g., dicer ribonuclease) found in cells. The RNAi response also features an endonuclease complex containing siRNA, commonly referred to as an RNA-induced silencing complex (RISC), which mediates the cleavage of single stranded RNA having a sequence complementary to the antisense region of the siRNA duplex. Cleavage of the target RNA (e.g., EAT-2 mRNA) takes place in the middle of the region complementary to the antisense strand of the siRNA duplex (Elbashir et al., 2001, Genes Dev., 15:188).

“Small interfering RNA” of the present invention refers to any nucleic acid molecule capable of mediating RNA interference “RNAi” or gene silencing (see for example, Bass, 2001, Nature, 411:428-429; Elbashir et al., 2001, Nature, 411:494-498; Kreutzer et al., International PCT publication No. WO 00/44895; Zernicka-Goetz et al., International PCT publication No. WO 01/36646; Fire, International PCT publication No. WO99/32619; Mello and Fire, International PCT publication No. WO01/29058; Deschamps-Depaillette, International PCT publication No. WO99/07409; Han et al., International PCT publication No. WO 2004/011647; Tuschl et al., International PCT publication No. WO 02/44321; and Li et al., International PCT publication No. WO 00/44914). For example, siRNA of the present invention are double stranded RNA molecules from about ten to about 30 nucleotides long that are named for their ability to specifically interfere with protein expression. In one embodiment, siRNA of the present invention are 12-28 nucleotides long, more preferably 15-25 nucleotides long, even more preferably 19-23 nucleotides long and most preferably 21-23 nucleotides long. Therefore preferred siRNA of the present invention are 12, 13, 14, 15, 16, 17, 18, 19, 20, 21, 22, 23, 24, 25, 26, 27, 28 nucleotides in length. As used herein, siRNA molecules need not to be limited to those molecules containing only RNA, but further encompass chemically modified nucleotides and non-nucleotides.

The length of one strand designates the length of a siRNA molecule. For example, a siRNA that is described as a 23 ribonucleotides long (a 23 mer) could comprise two opposite strands of RNA that anneal together for 21 contiguous base pairing. The two remaining ribonucleotides on each strand would form what is called an “overhang”. In a particular embodiment, the siRNA of the present invention contains two strands of different lengths. In this case, the longer strand designates the length of the siRNA. For example, a dsRNA containing one strand that is 20 nucleotides long and a second strand that is 19 nucleotides long is considered a 20 mer.

siRNAs that comprise an overhang are desirable. The overhang may be at the 3′ or 5′ end. Preferably, the overhangs are at the 3′ end of an RNA strand. The length of an overhang may vary but preferably is about 1 to 5 nucleotides long. Generally, 21 nucleotides siRNA with two nucleotides 3′-overhang are the most active siRNAs.

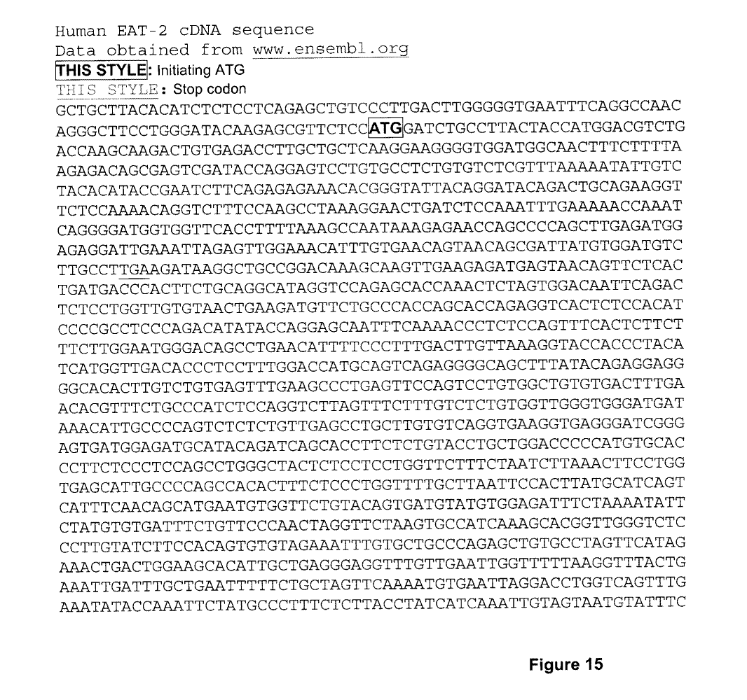

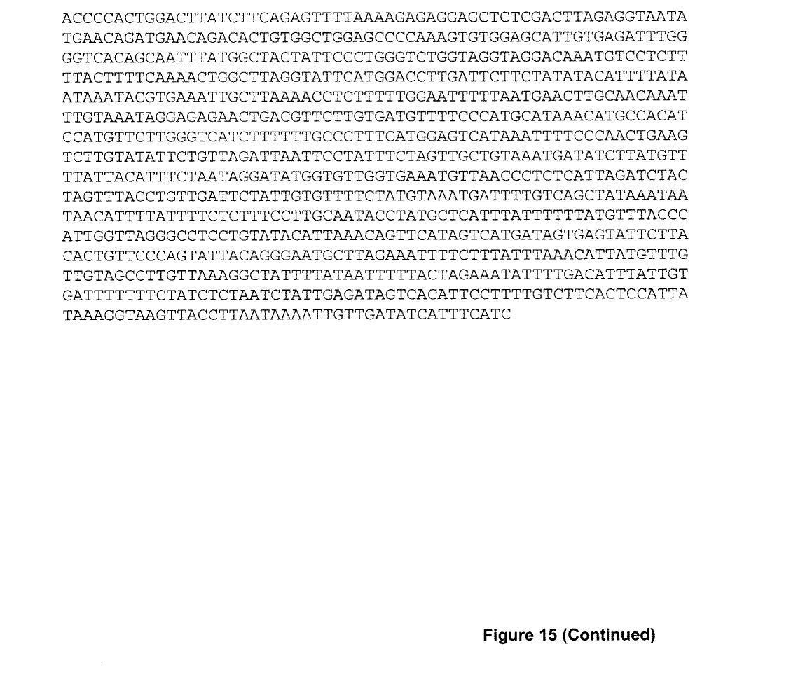

siRNA of the present invention are designed to decrease EAT-2 expression in a target cell by RNA interference. siRNA of the present invention comprise a sense region and an antisense region wherein the antisense region comprises a sequence complementary to an EAT-2 mRNA sequence (e.g., FIG. 15) and the sense region comprises a sequence complementary to the antisense sequence of EAT-2 mRNA. A siRNA molecule can be assembled from two nucleic acid fragments wherein one fragment comprises the sense region and the second fragment comprises the antisense region of siRNA molecule. The sense region and antisense region can also be covalently connected via a linker molecule. The linker molecule can be a polynucleotide linker or a non-polynucleotide linker.

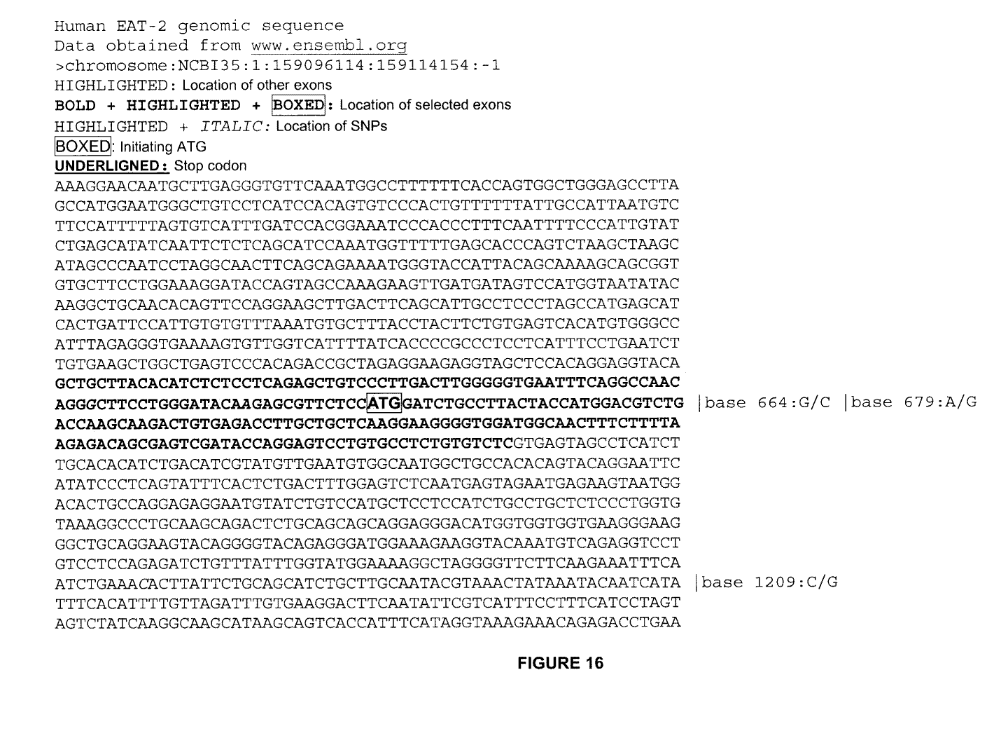

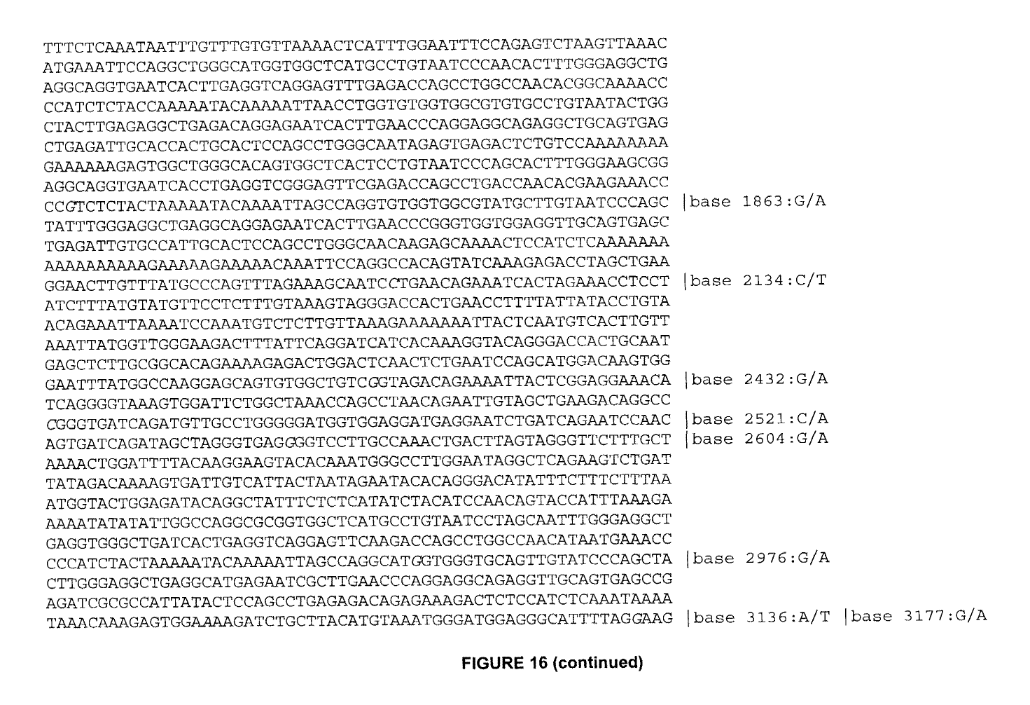

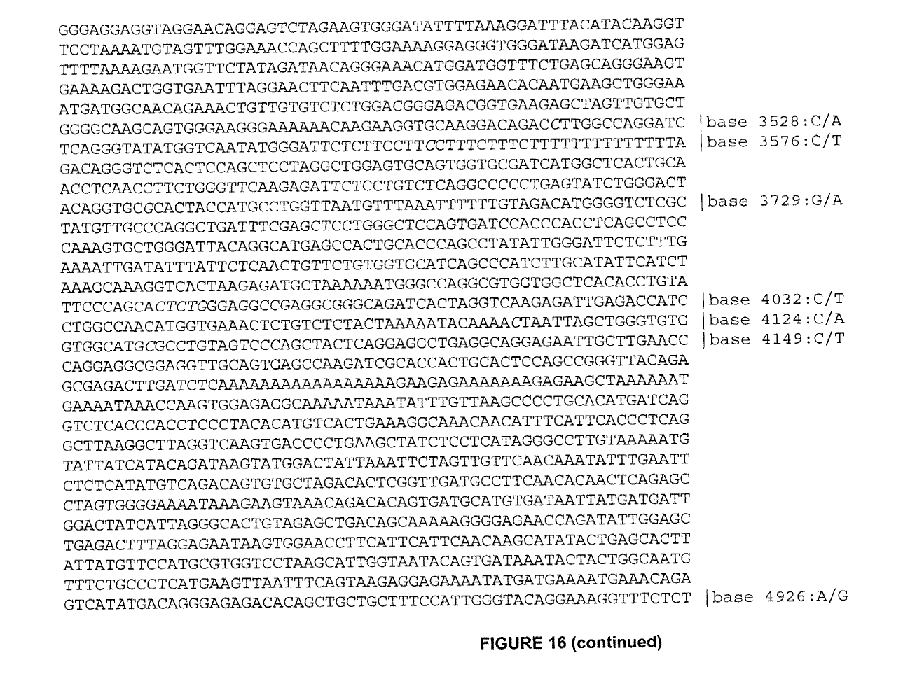

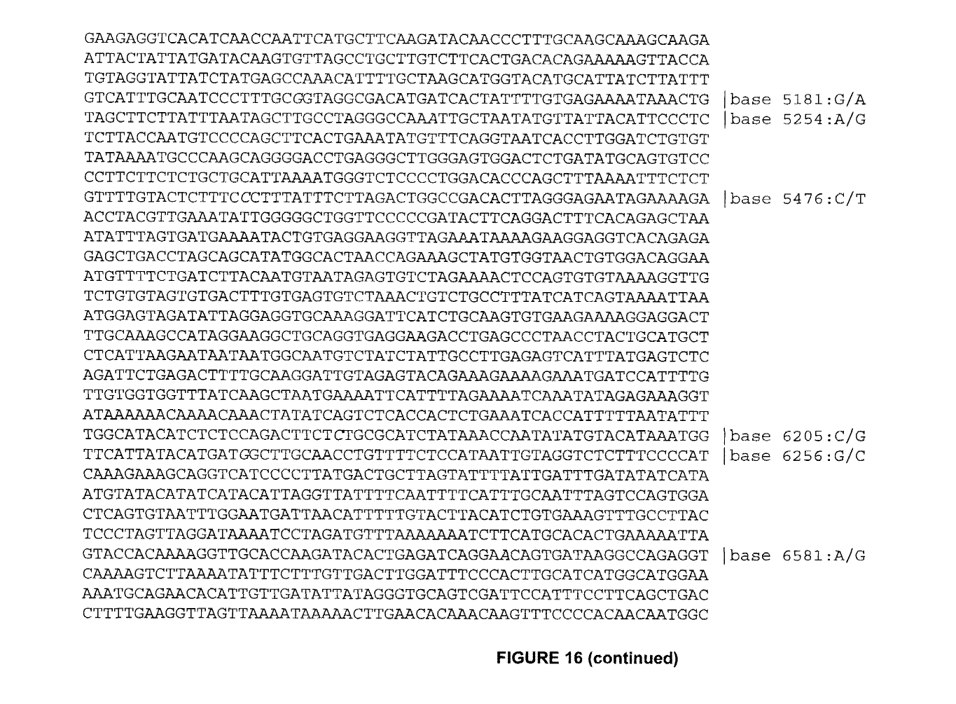

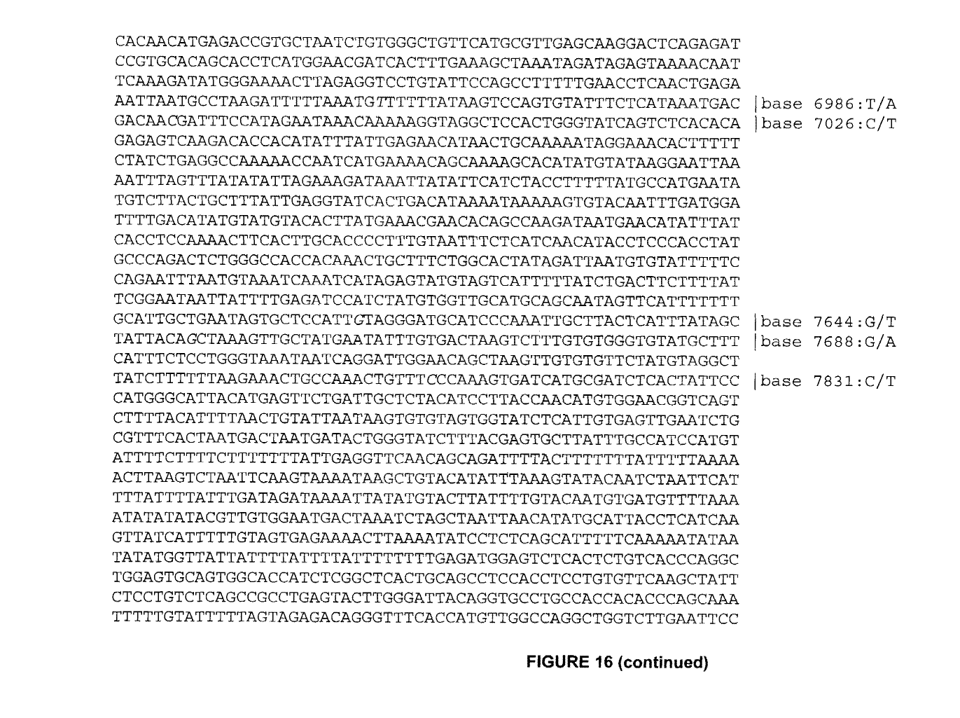

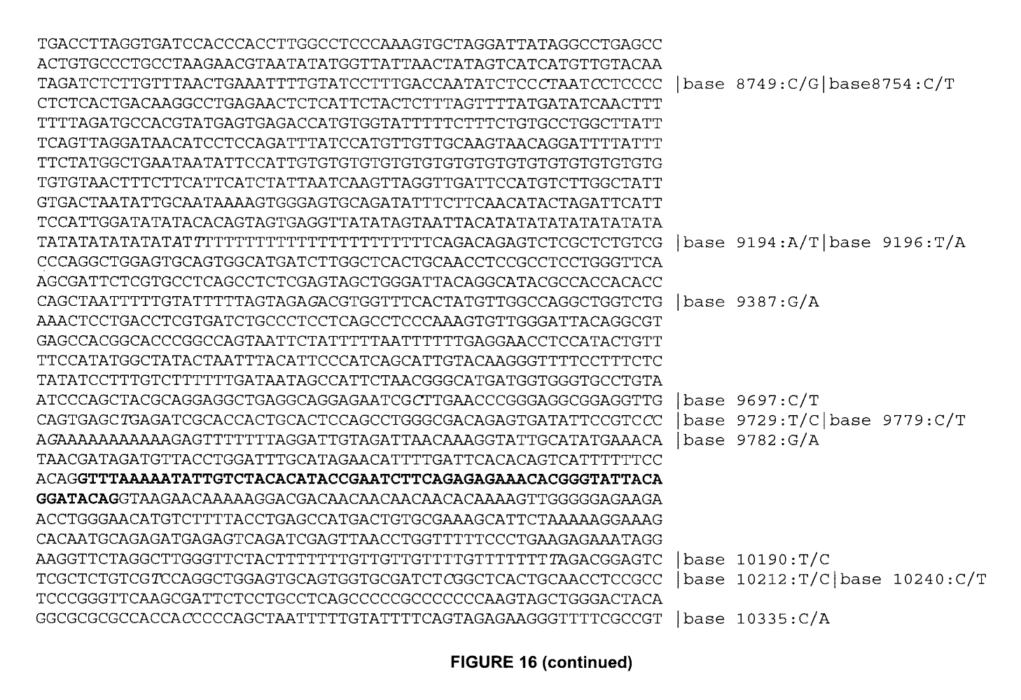

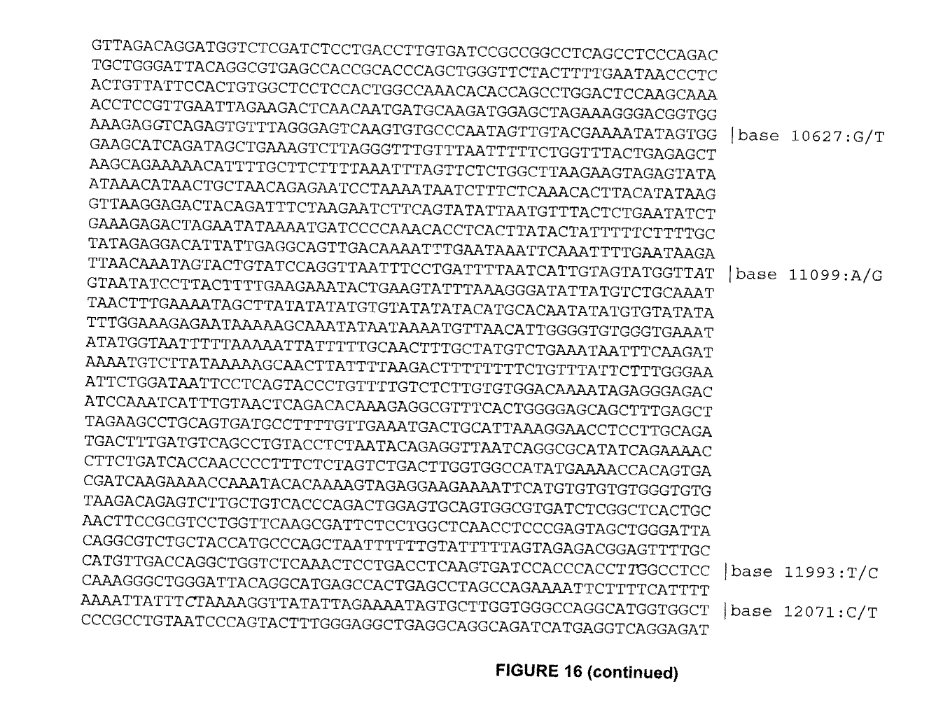

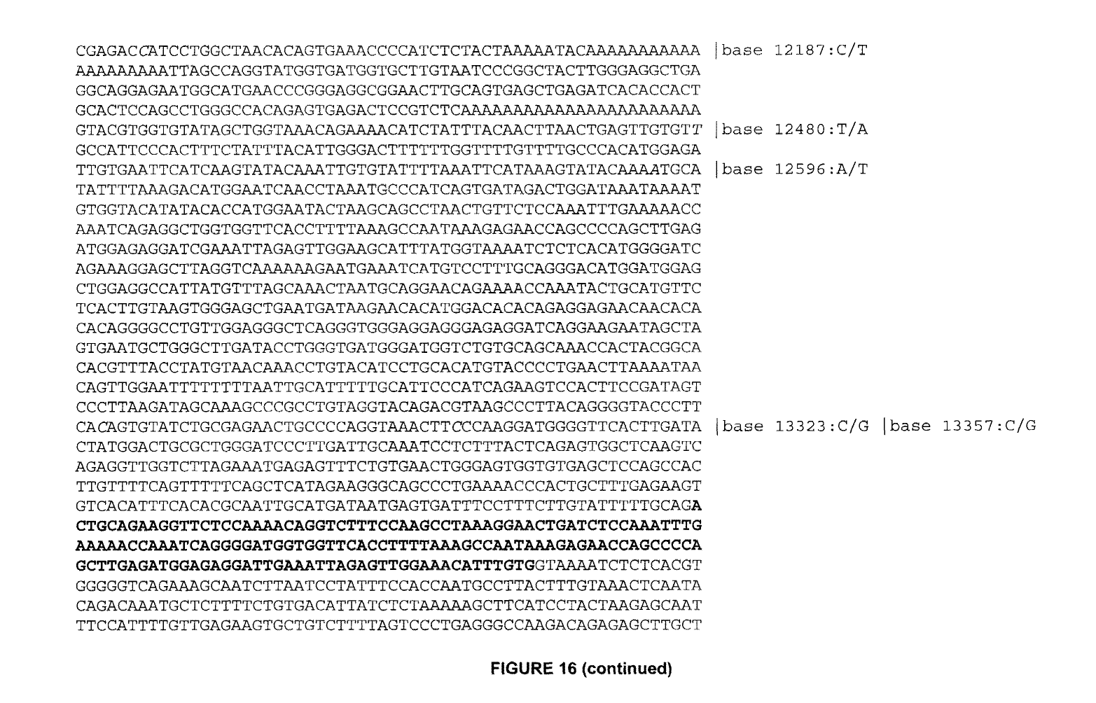

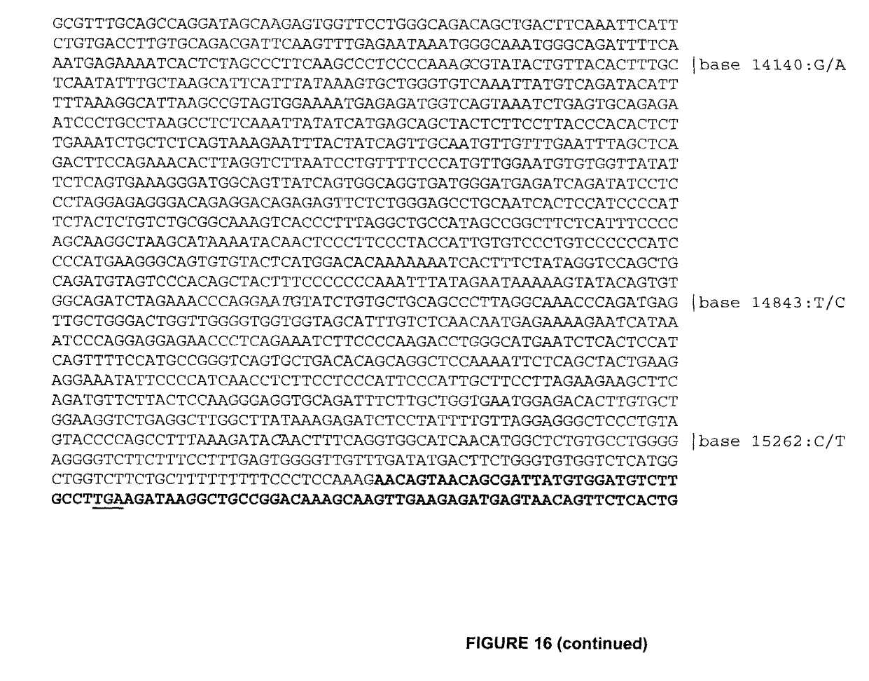

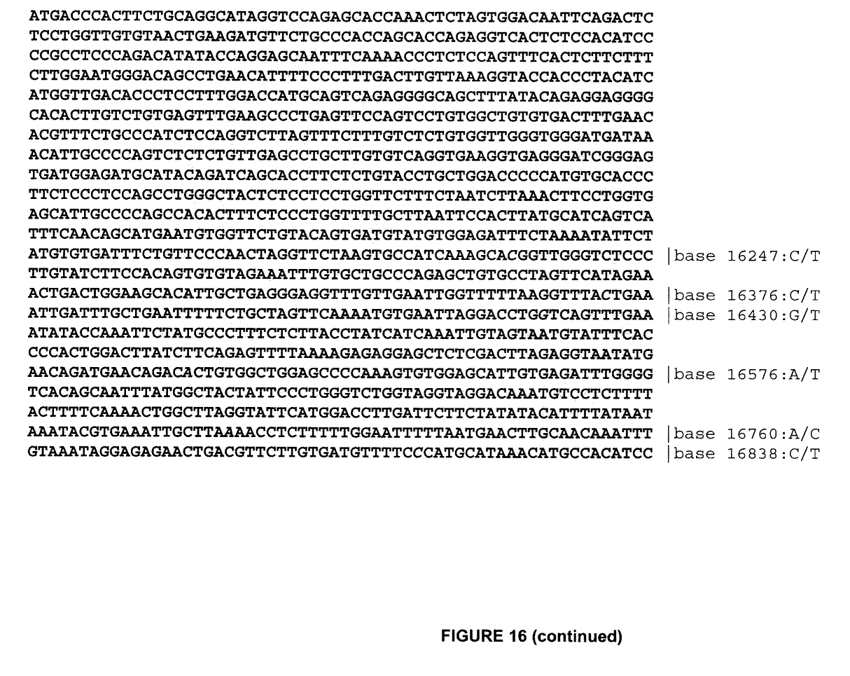

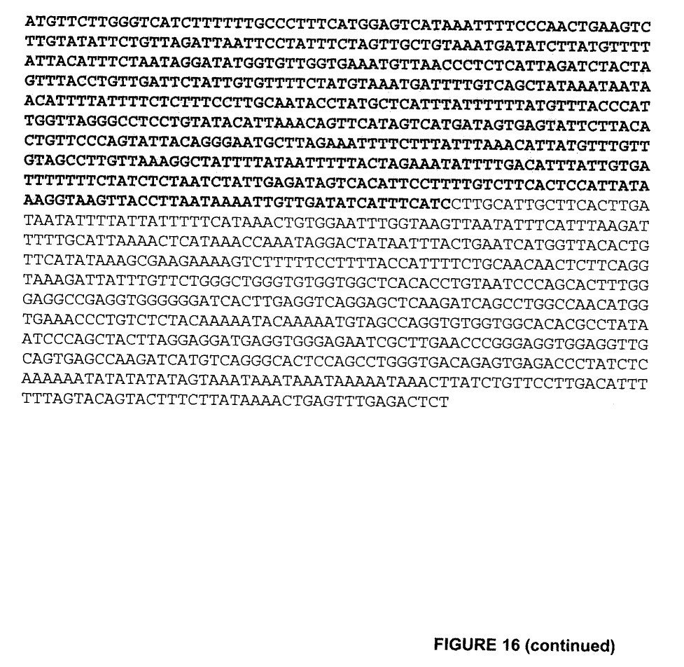

In one embodiment, the present invention features a siRNA molecule having RNAi activity against EAT-2 RNA, wherein the siRNA molecule comprises a sequence complementary to any RNA having an EAT-2 encoding sequence. A siRNA molecule of the present invention can comprise any contiguous EAT-2 sequence (e.g., 19-23 contiguous nucleotides present in a EAT-2 sequence such as in FIG. 15). In the particular case where alternate splicing produces a family of transcripts that are distinguished by specific exons, the present invention can be used to inhibit gene expression of a particular gene family member through the targeting of the appropriate exon(s) (e.g., to specifically knock down the expression of a EAT-2 particular transcript) or of the full length transcript (FIGS. 15 and 16).

siRNAs of the present invention comprise a ribonucleotide sequence that is at least 80% identical to an EAT-2 ribonucleotide sequence. Preferably, the siRNA molecule is at least 90%, at least 95% (e.g., 95, 96, 97, 99, 99, 100%), at least 98% (e.g., 98, 99, 100%) or at least 99% (e.g., 99, 100%) identical to the ribonucleotide sequence of the target gene (e.g., EAT-2 RNA). siRNA molecule with insertion, deletions, or single point mutations relative to the target may also be effective. Mutations that are not in the center of the siRNA molecule are more tolerated. Tools to assist siRNA design are well known in the art and readily available to the public. For example, a computer-based siRNA design tool is available on the Internet at www.dharmacon.com or on the web site of several companies that offer the synthesis of siRNA molecules.

In one embodiment, the siRNA molecules of the present invention are chemically modified to confer increased stability against nuclease degradation but retain the ability to bind to the target nucleic acid that is present in a cell. Modified siRNAs of the present invention comprise modified ribonucleotides, and are resistant to enzymatic degradation such as RNAse degradation, yet they retain their ability to reduce EAT-2 expression in a target cell. The siRNA may be modified at any position of the molecule so long as the modified siRNA is still capable of binding to the target sequence and is more resistant to enzymatic degradation. Modifications in the siRNA may be in the nucleotide base (i.e., purine or pyrimidine), the ribose or phosphate.

More specifically, the siRNA may be modified in at least one purine, in at least one pyrimidine or a combination thereof. Generally, all purines (adenosine or guanine) or all pyrimidine (cytosine or uracyl) or a combination of all purines and all pyrimidines of the siRNA are modified. Ribonucleotides on either one or both strands of the siRNA may be modified.

Non-limiting examples of chemical modification that can be included in an siRNA molecule include phosphorothioate internucleotide linkages (see US 2003/0175950), 2′-O-methyl ribonucleotides, 2′-O-methyl modified ribonucleotides, 2′-deoxy-2′-fluoro ribonucleotides, 2′-deoxy-2′-fluoro modified pyrimidines nucleotides, 5-C-methyl nucleotides and deoxyabasic residue incorporation. The ribonucleotides containing pyrimidine bases can be modified at the 2′ position of the ribose residue. A preferable modification is the addition of a molecule from the halide chemical group such as fluorine. Other chemical moieties such as methyl, methoxymethyl and propyl may also be added as modifications (see International PCT publication No. WO2004/011647). These chemical modifications, when used in various siRNA constructs, are shown to preserve RNAi activity in cells while at the same time, dramatically increasing their stability in cells or serum. Chemical modifications of the siRNA of the present invention can also be used to improve the stability of the interaction with the target RNA sequence.

siRNAs of the present invention may also be modified by the attachment of at least one receptor binding ligand to the siRNA. Receptor binding ligand can be any ligand or molecule that directs the siRNA of the present invention to a specific target cell (e.g., NK cells, macrophage, dendritic cells). Such ligands are useful to direct delivery of siRNA to a target cell in a body system, organ or tissue of a subject such as NK cells. Receptor binding ligand may be attached to one or more siRNA ends, including any combination of 5′ or 3′ ends. The selection of an appropriate ligand for delivering siRNAs depends on the cells, tissues or organs that are targeted and is considered to be within the ordinary skill of the art. For example, to target a siRNA to hepatocytes, cholesterol may be attached at one or more ends, including 3′ and 5′ ends. Other conjugates such as other ligands for cellular receptors (e.g., peptides derived from naturally occurring protein ligands), protein localization sequences (e.g., ZIP code sequences), antibodies, nucleic acid aptamers, vitamins and other cofactors such as N-acetylgalactosamine and folate, polymers such as polyethyleneglycol (PEG), polyamines (e.g., spermine or spermidine) and phospholipids can be linked (directly or indirectly) to the siRNA molecule for improving its bioavailability.

siRNAs can be prepared in a number of ways well known in the art, such as by chemical synthesis, T7 polymerase transcription, or by treating long double stranded RNA (dsRNA) prepared by one of the two previous methods with Dicer enzyme. Dicer enzyme create mixed population of dsRNA from about 21 to 23 base pairs in length from double stranded RNA that is about 500 base pairs to about 1000 base pairs in size. Dicer can effectively cleave modified strands of dsRNA, such as 2′-fluoromodified dsRNA (see WO2004/011647).

In one embodiment, vectors are employed for producing siRNAs by recombinant techniques. Thus, for example, a DNA segment encoding a siRNA derived from an EAT-2 sequence (e.g., FIG. 15) may be included in any one of a variety of expression vectors for expressing any DNA sequence derived from an EAT-2 sequence. Such vectors include synthetic DNA sequences (e.g., derivatives of SV40, bacterial plasmids, baculovirus, yeast plasmids, viral DNA such as vaccinia, fowl pox virus, adenovirus, lentivirus, retrovirus, adeno-associated virus, alphavirus etc.), chromosomal and non-chromosomal vectors. Any vector may be used in accordance with the present invention as long as it is replicable and viable in the desired host. The DNA segment in the expression vector is operably linked to an appropriate expression control sequence (e.g., promoter) to direct siRNA synthesis. Preferably, the promoters of the present invention are from the type III class of RNA polymerase III promoters (e.g., U6 and H1 promoters). The promoters of the present invention may also be inducible, in that the expression may be turned on or turned off (e.g., tetracycline-regulatable system employing the U6 promoter to control the production of siRNA targeted to EAT-2).

In a particular embodiment, the present invention utilizes a vector wherein a DNA segment encoding the sense strand of the RNA polynucleotide is operably linked to a first promoter and the antisense strand of the RNA polynucleotide is operably linked to a second promoter (i.e., each strand of the RNA polynucleotide is independently expressed).

In another embodiment, the DNA segment encoding both strands of the RNA polynucleotide is under the control of a single promoter. In a particular embodiment, the DNA segment encoding each strand is arranged on the vector with a loop region connecting the two DNA segments (e.g., sense and antisense sequences), where the transcription of the DNA segments and loop region creates one RNA transcript. When transcribed, the siRNA folds back on itself to form a short hairpin capable of inducing RNAi. The loop of the hairpin structure is preferably from about 4 to 6 nucleotides in length. The short hairpin is processed in cells by endoribonucleases which remove the loop thus forming a siRNA molecule. In this particular embodiment, siRNAs of the present invention comprising a hairpin or circular structure are about 35 to about 65 nucleotides in length (e.g., 35, 36, 37, 38, 49, 40, 41, 42, 43, 44, 45, 46, 47, 48, 49, 50, 51, 52, 53, 54, 55, 56, 57, 58, 59, 60, 61, 63, 64, 65 nucleotides in length), preferably between 40 and 64 nucleotides in length comprising for example about 18, 19, 20, 21, 22, or 23, 24, 25 base pairs.

In yet a further embodiment, the vector of the present invention comprises opposing promoters. For example, the vector may comprise two RNA polymerase III promoters on either side of the DNA segment (e.g., a specific EAT-2 DNA segment) encoding the sense strand of the RNA polynucleotide and placed in opposing orientations, with or without a transcription terminator placed between the two opposing promoters.

Non-limiting examples of expression vectors used for siRNA expression are described in Lee et al., 2002, Nature Biotechnol., 19:505; Miyagishi and Taira, 2002, Nature Biotechnol., 19:497; Pau et al., 2002, Nature Biotechnol., 19:500 and Novina et al., 2002, Nature Medicine, July 8(7):681-686).

### Antisense RNAs

The present invention also features antisense nucleic acid molecules which can be used for example to decrease or abrogate the expression of EAT-2 to increase innate immune cell functions. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845, and U.S. Pat. No. 5,593,974. Antisense nucleic acid molecules according to the present invention can be derived from the nucleic acid sequences and modified in accordance with well-known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In one embodiment, antisense approach of the present invention involves the design of oligonucleotides (either DNA or RNA) that are complementary to EAT-2 mRNA. The antisense oligonucleotides bind to EAT-2 mRNA and prevent its translation. Absolute complementarity, although preferred, is not a definite prerequisite. One skilled in the art can identify a certain tolerable degree of mismatch by use of standard methods to determine the melting point of the hybridized antisense complex. In general, oligonucleotides that are complementary to the 5′untranslated region (up to the first AUG initiator codon) of EAT-2 mRNA should work more efficiently at inhibiting translation and production of EAT-2 protein. However, oligonucleotides that are targeted to a coding portion of the sequence may produce inactive truncated protein or diminish the efficiency of translation thereby lowering the overall expression of EAT-2 protein in a cell. Antisense oligonucleotides targeted to the 3′ untranslated region of messages have also proven to be efficient in inhibiting translation of targeted mRNAs (Wagner, R. (1994), Nature, 372:333-335). The EAT-2 antisense oligonucleotides of the present invention are less than 100 nucleotides in length, particularly, less than 50 nucleotides in length and more particularly less than 30 nucleotides in length. Generally, effective antisense oligonucleotides are at least 15 or more oligonucleotides in length.

The antisense oligonucleotides of the present invention can be DNA, RNA, Chimeric DNA-RNA analogue, and derivatives thereof (see Inoue et al. (1987). Nucl. Acids. Res. 15: 6131-6148; Inoue et al. (1987), FEBS lett. 215: 327-330; Gauthier at al. (1987), Nucl. Acids, Res. 15: 6625-6641.). As mentioned above, antisense oligonucleotides of the present invention may include modified bases or sugar moiety. Examples of modified bases include xanthine, hypoxanthine, 2-methyladenine, N6-isopentenyladenine, 2-methylguanine, 3-methylcytosine, 5-methylcytosine, N6-adenine, 7-methyguanine, 5-fluorouracil, 5-chlorouracil, 5-bromouracil, 5-iodouracyl, 5-carboxymethylaminomethyluracil, 5-methoxycarboxymethyluracil, queosine, 4-thiouracil and 2,6-diaminopurine. Examples of modified sugar moieties include hexose, xylulose, arabinose and 2-fluoroarabinose. The antisense oligonucleotides of the present invention may also include modified phosphate backbone such as methylphosphonate, phosphoramidate, phosphoramidothioates, phosphordiamidate and alkyl phosphotriesters. The synthesis of modified oligonucleotides can be done according to methods well known in the art.

Once an antisense oligonucleotide or siRNA is designed, its effectiveness can be appreciated by conducting in vitro studies that assess the ability of the antisense to inhibit gene expression (e.g., EAT-2 protein expression). Such studies ultimately compare the level of EAT-2 RNA or protein with the level of a control experiment (e.g., an oligonucleotide which is the same as that of antisense experiment but being a sense oligonucleotide or an oligonucleotide of the same size as the antisense oligonucleotide but that does not bind to a specific EAT-2 sequence).

### Increase EAT-2 Expression

In particular conditions, it might be useful to stimulate or increase the expression of EAT-2 in cells such as innate immune cells. This could decrease the ability of cells to modulate an inappropriate immune response useful for example in the treatment of autoimmune diseases such as lupus and rheumatoid arthritis and diseases caused by insufficient EAT-2 expression.

Thus, in one particular embodiment, the present invention features gene therapy methods to increase EAT-2 expression in cells. The EAT-2 sequences used in the gene therapy method of the present invention may be either a full-length EAT-2 nucleic acid sequence (e.g., FIGS. 15 and 16) or be limited to sequences encoding the biologically active domains of a EAT-2 protein. Thus, any EAT-2 sequence having at least one conserved biological activity of native EAT-2 protein may be used in accordance with the present invention (e.g., increase IFN-γ production and increase cytotoxic activity of NK cells). The EAT-2 sequence may be under the control of its natural promoter or under the control of other strong promoters allowing either general expression or cell-type or tissue specific expression.

### Gene Therapy Methods

In the gene therapy methods of the present invention, an exogenous sequence (e.g., an EAT-2 or ERT gene or cDNA sequence, an EAT-2 or ERT siRNA or antisense nucleic acid) is introduced and expressed in an animal (preferably a human) to supplement, replace or inhibit a target gene (i.e., EAT-2 or ERT gene). or to enable target cells to produce a protein (e.g., a EAT-2 chimeric protein to target a specific molecule to innate immune cells) having a prophylactic or therapeutic effect toward autoimmune diseases, cancers, infectious diseases and other EAT-2 related diseases.

Non virus-based and virus-based vectors (e.g., adenovirus- and lentivirus-based vectors) for insertion of exogenous nucleic acid sequences into eukaryotic cells are well known in the art and may be used in accordance with the present invention. Virus-based vectors (and their different variations) for use in gene therapy are well known in the art. In virus-based vectors, parts of a viral gene are replaced by the desired exogenous sequence so that a viral vector is produced. Viral vectors are very often designed to no longer be able to replicate due to DNA manipulations.

In one specific embodiment, lentivirus derived vectors are used to target an EAT-2 sequence (e.g., siRNA, antisense, nucleic acid encoding a partial or complete EAT-2 protein) into specific target cells (e.g., innate immune cells such as NK cells). These vectors have the advantage of infecting quiescent cells (for example see U.S. Pat. No. 6,656,706; Amado et al., 1999, Science 285: 674-676).

In addition to an EAT-2 nucleic acid sequence, siRNA or antisense, the vectors of the present invention may contain a gene that acts as a marker by encoding a detectable product.

One way of performing gene therapy is to extract cells from a patient, infect the extracted cells with a viral vector and reintroduce the cells back into the patient. A selectable marker may or may not be included to provide a means for enriching the infected or transduced cells. Alternatively, vectors for gene therapy that are specially formulated to reach and enter target cells may be directly administered to a patient (e.g., intravenously, orally etc.).

The exogenous sequences (e.g., antisense RNA, siRNA, a EAT-2 sequence, or EAT-2 targeting vector for homologous recombination) may be delivered into cells that express EAT-2 according to well known methods. Apart from infection with virus-based vectors, examples of methods to deliver nucleic acid into cells include DEAE dextran lipid formulations, liposome-mediated transfection, CaCl₂-mediated transfection, electroporation or using a gene gun. Synthetic cationic amphiphilic substances, such as dioleoyloxypropyl-methylammonium bromide (DOTMA) in a mixture with dioleoyl-phosphatidylethanolamine (DOPE), or lipopolyamine (Behr, Bioconjugate Chem., 1994 5:382), have gained considerable importance in charged gene transfer. Due to an excess of cationic charge, the substance mixture complexes with negatively charged genes and binds to the anionic cell surface. Other methods include linking the exogenous oligonucleotide sequence (e.g., siRNA, antisense, EAT-2 sequence encoding an EAT-2 protein, EAT-2 targeting vector for homologous recombination, etc.) to peptides or antibodies that especially bind to receptors or antigens at the surface of a target cell. U.S. Pat. No. 6,358,524 describes target cell-specific non-viral vectors for inserting at least one gene into cells of an organism. The method describes the use of non-viral carriers that are cationized to enable them to complex with the negatively charged DNA. Moreover, the method also includes the use of a ligand (e.g., a monoclonal antibody or fragment thereof that is specific in this particular case for membrane antigen present on the surface of innate immune cells, e.g., NK cells) that can specifically bind to the desired target cell in order to enter it.

To achieve high cellular concentration of the EAT-2 antisense nucleic acid or small inhibitor RNAs of the present invention, an effective method utilizes a recombinant DNA construct in which the nucleic acid sequence is placed under a strong promoter and the entire construct is targeted into the cell. Such promoter may constitutively or inducibly produce the EAT-2 sequence encoding EAT-2 protein (or portion thereof), antisense RNA or siRNA of the present invention.

### Assays to Identify Modulators of EAT-2 and ERT

In order to identify modulators of EAT-2 (or ERT) activity in innate immune cells (e.g., NK cells, macrophages and dendritic cells), several screening assays aiming at reducing, abrogating or stimulating a functional activity of EAT-2 (or ERT) in cells can be designed in accordance with the present invention.

One possible way is by screening libraries of candidate compounds for inhibitors of the phosphorylation of EAT-2 (or ERT). Other possibilities include screening for compounds that inhibit the interaction of EAT-2 (or ERT) with SLAM-related receptors (e.g., 2B4), kinases, or other effector molecules interacting with EAT-2 (or ERT). Inhibitors of other EAT-2 (or ERT) functional activities may also be identified in accordance with the present invention, as long as such functional activities are related to EAT-2 (or ERT) function in innate immune cells. In addition, libraries of candidate compounds may also be screened for stimulators of EAT-2 (or ERT) activity (e.g., compounds which increase the phosphorylation status of EAT-2, stabilizes its interaction with SLAM related receptors or effector proteins, etc.). Screening assays and compounds which directly or indirectly modulate (i.e., decrease or increase) EAT-2 or ERT expression in cells are also encompassed by the present invention.

For example, combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection may be used in order to identify modulators of EAT-2 or ERT biological activity. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261:1303; Carrell et al. (1994) Angew. Chem, Int. Ed Engl. 33:2059; and ibid 2061; and in Gallop et al. (1994). Med Chem. 37:1233. Libraries of compounds may be presented in solution (e.g., Houghten (1992) Biotechniques 13:412-421) or on beads (Lam (1991) Nature 354:82-84), chips (Fodor (1993) Nature 364:555-556), bacteria or spores (Ladner U.S. Pat. No. 5,223,409), plasmids (Cull et al. (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) supra; Erb et al. (1994) supra; Zuckermann et al. (1994) supra; Cho et al. (1993) supra; Carrell et al. (1994) supra, or luciferase, and the enzymatic label detected by determination of conversion of an appropriate substrate to product. The choice of a particular combinatorial library depends on the specific EAT-2 or ERT activity that needs to be modulated.

The SH2 domain of EAT-2 has been identified as one of the functional regions of EAT-2. Thus, to reduce EAT-2 activity in cells, the present invention concerns the use of assays allowing rapid identification of small molecules that inhibit EAT-2 binding activity to SLAM related receptors. Similarly, screening assays that will permit rapid identification of small molecules that inhibit EAT-2 phosphorylation or EAT-2 binding to effector molecules are encompassed by the present invention.

All methods and assays of the present invention may be developed for low-throughput, high-throughput, or ultra-high throughput screening formats. Of course, methods and assays of the present invention are amenable to automation. Automation and low-throughput, high-throughput, or ultra-high throughput screening formats are possible for the screening of agents which modulates the level and/or activity of EAT-2.

Generally, high throughput screens for EAT-2 or ERT modulators i.e. candidate or test compounds or agents (e.g., peptides, peptidomimetics, small molecules, antisense RNA, Ribozyme, or other drugs) may be based on assays which measure a biological activity of EAT-2 or ERT. The invention therefore provides a method (also referred to herein as a “screening assay”) for identifying modulators, which have an inhibitory effect on, for example, an EAT-2 biological activity or expression thereof, or which binds to or interacts with EAT-2 proteins, or which has a stimulatory or inhibitory effect on, for example, the production of IFN-γ by NK cells or the ability of NK cells to kill target cells.

The assays described above may be used as initial or primary screens to detect promising lead compounds for further development. Often, lead compounds will be further assessed in additional, different screens. Therefore, this invention also includes secondary EAT-2 screens which may involve assays utilizing mammalian cell lines expressing EAT-2.

Tertiary screens may involve the study of the identified modulators in the appropriate rat and mouse models. Accordingly, it is within the scope of this invention to further use an agent identified as described herein in an appropriate animal model. For example, a test compound identified as described herein (e.g., an EAT-2 inhibiting agent, an antisense EAT-2 nucleic acid molecule, an EAT-2 siRNA, an EAT-2 antibody etc.) can be tested in the transgenic mice overexpressing EAT-2 of the present invention to determine the efficacy, toxicity, or side effects of treatment with such an agent. Furthermore, this invention pertains to uses of novel agents identified by the above-described screening assays for treatment of cancers, infectious diseases and autoimmune diseases, as described herein.

The present invention is illustrated in further details by the following non-limiting examples.

### Example 1

### Identification of ERT, a Novel Sap-Related Adaptor Expressed in Mouse NK Cells

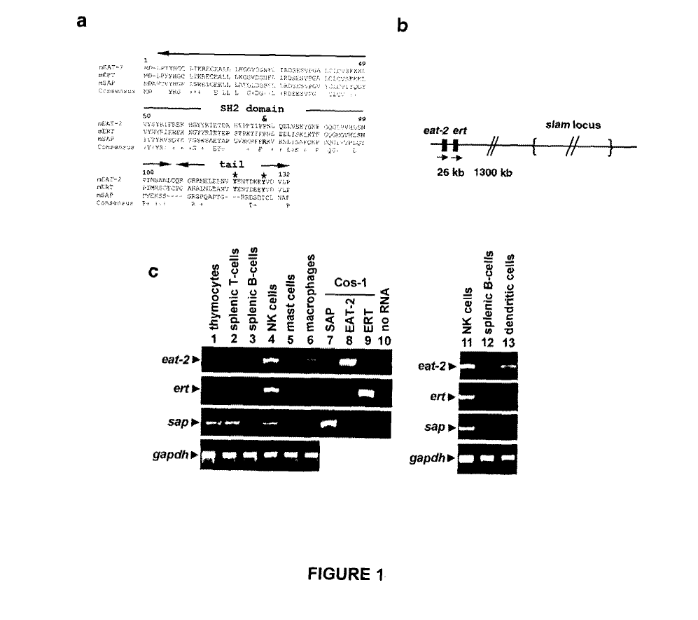

During the process of analyzing the sequence of mouse eat-2 using public databases, a mouse cDNA sequence (accession number BB617320) that may encode a third member of the SAP family (FIG. 1a; data not shown) was discovered. The polypeptide translated from this cDNA, named ERT (for EAT-2-related transducer), is most closely related to EAT-2 (82% amino-acid identity). This homology extends to the SH2 domain and the carboxyl-terminal region. ERT is less similar to SAP (36% amino-acid identity). This is especially true for the carboxyl-terminal domain, which shows no conservation between EAT-2/ERT and SAP. Examination of the mouse genomic database showed that the ert gene is located approximately 26 kilobases (kb) away from the eat-2 gene on chromosome 1 (FIG. 1b). Since ert exhibits the same exon-intron structure as eat-2 and sap, it likely represents a third member of the sap family.

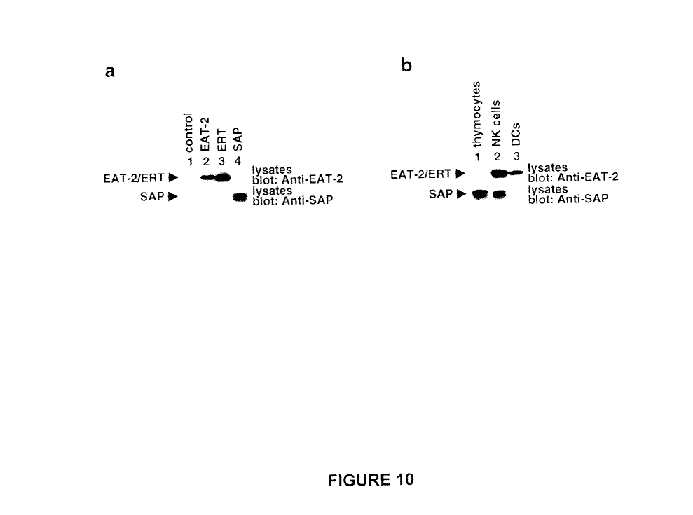

To determine the expression pattern of eat-2 and ert, RNA was isolated from mouse immune cells, and transcript levels were determined by reverse transcriptase (RT)-polymerase chain reactions (PCRs) (FIG. 1c). eat-2 RNA (first panel) was easily detectable in NK cells (lanes 4 and 11), macrophages (lane 6) and dendritic cells (DCs) (lane 13). However, it was not found in thymocytes (lane 1), splenic T-cells (lane 2), mast cells (lane 5) and B-cells (lanes 3 and 12). Contrary to eat-2, ert (second panel) was detected only in NK cells (lanes 4 and 11). In keeping with previous reports^{7,15,23,24}, sap (third panel) was present in thymocytes (lane 1), splenic T-cells (lane 2) and NK cells (lanes 4 and 11). The specificity of these PCR reactions was demonstrated by testing RNA samples from Cos-1 cells transfected with mouse sap (lane 7), eat-2 (lane 8) or ert (lane 9) cDNAs, respectively. The expression patterns of the SAP-related adaptors were also confirmed by protein immunoblotting (FIG. 10).

### Example 2

### Enhanced NK Cell Functions in Mice Lacking EAT-2 or ERT, but not SAP

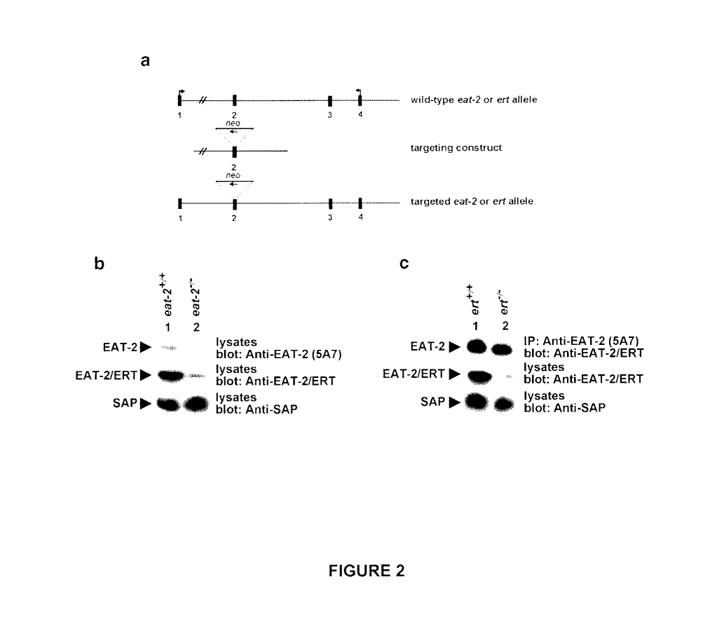

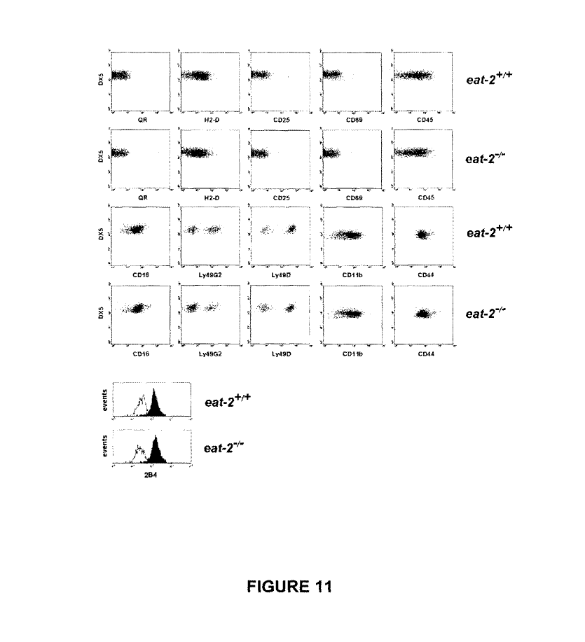

In the light of the close relationship between EAT-2 and ERT and since both are expressed in innate immune cells such as NK cells, the inventors decided to analyze the function of these two molecules in NK cells. For this purpose, mice lacking EAT-2 or ERT were generated by homologous recombination. After transfection of the constructs depicted in FIG. 2a in mouse embryonic stem (ES) cells, 129/Sv mice homozygous for disruption of the eat-2 or ert gene were created (data not shown). Immunoblotting of NK cell lysates with an antibody specific against EAT-2 confirmed the lack of EAT-2 expression in eat-2^{-/-} mice (FIG. 2b, first panel, lane 2). Similarly, immunoblotting of lysates with an antibody that recognized both ERT and EAT-2 demonstrated a striking reduction in the abundance of immunoreactive products in ert^{-/-} animals (FIG. 2c, second panel, lane 2) (unfortunately, at the time of performing these experiments, the inventors did not have ERT-specific antibodies). Expression of the other SAP-related adaptors was not affected by these mutations (FIGS. 2b and c). Mice lacking EAT-2 or ERT were apparently healthy and fertile (data not shown). Moreover, they did not exhibit any obvious alteration in NK cell development in vivo (FIG. 11) or in vitro (data not shown).

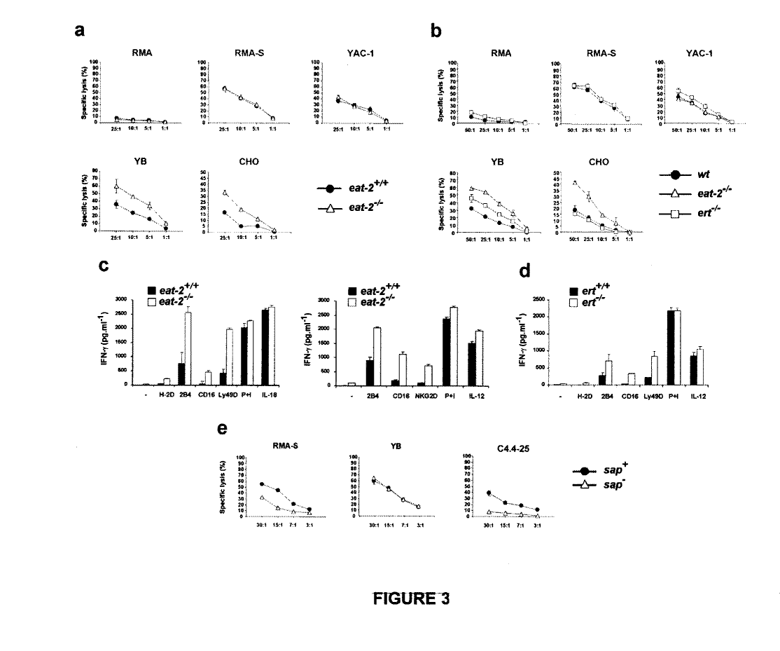

To study the impact of EAT-2 deficiency on mature NK functions, IL-2-activated NK cells were obtained from eat-2^{+/+} and eat-2^{-/-} animals, and the ability of NK cells to mediate natural cytotoxicity was evaluated (FIG. 3a). NK cells were incubated with various ⁵¹Cr-labeled target cells, and target cell killing was measured by monitoring the release of ⁵¹Cr in the supernatant. Compared to wild-type NK cells, EAT-2-deficient NK cells had an increased ability to kill target cells such as CHO and YB. This effect was seen in several independent experiments (data not shown). Interestingly, natural killing of these two xenogeneic targets is highly dependent on the capacity of the stimulatory receptor Ly49D to recognize class I MHC on the target cells. Cytotoxicity against other targets like RMA, RMA-S and YAC-1 was not significantly altered by EAT-2 deficiency.

Similar experiments were performed using NK cells from ERT-deficient mice (FIG. 3b). As was the case for EAT-2 deficiency, lack of ERT expression resulted in an augmentation of the ability to kill YB cells, although the increase was less pronounced than that seen in eat-2^{-/-} NK cells. Moreover, it did not affect killing of RMA, RMA-S and YAC-1. Unlike EAT-2 deficiency, though, defective ERT expression did not enhance the capacity of NK cells to destroy CHO cells.

Next, the impact of EAT-2 or ERT deficiency on the ability of NK cells to produce IFN-γ was tested. IL-2-activated NK cells were stimulated with antibodies directed against various stimulatory NK receptors, and the subsequent production of IFN-γ was measured by enzyme-linked immunosorbent assay (ELISA) (FIGS. 3c and d). When compared with eat-2^{+/+} NK cells, NK cells from eat-2^{-/-} mice demonstrated a pronounced increase (4- to 5-fold) in IFN-γ secretion in response to stimulation of CD16, NKG2D or Ly49D (FIG. 3c). A smaller (˜2- to 3-fold), albeit reproducible, increase in the production of IFN-γ was also observed in response to 2B4 stimulation (FIG. 3c). Furthermore, an augmentation of IFN-γ secretion was seen in unstimulated cells. These effects did not reflect a global enhancement of NK responsiveness, as the response to IL-12, IL-18 or phorbol myristate acetate (PMA) and ionomycin was either minimally elevated or not affected by EAT-2 deficiency. It is possible that the slightly enhanced IFN-γ secretion seen in response to IL-12, IL-18 or PMA plus ionomycin simply reflected the augmentation of IFN-γ production observed in unstimulated EAT-2-deficient NK cells. As was the case for EAT-2-deficient NK cells, ERT-deficient NK cells also showed an enhanced response to CD16, Ly49D, NKG2D and, to a lesser extent, 2B4 (FIG. 3d; data not shown). Furthermore, responsiveness to IL-12 or PMA plus ionomycin was not affected.

To determine if the increase in NK responsiveness caused by EAT-2 or ERT deficiency was specific to ablation of these proteins, NK functions were also studied in sap⁻ mice (FIG. 3e) (Bloch-Queyrat et al., submitted). Unlike eat-2^{-/-} and ert^{-/-} NK cells, sap⁻ NK cells demonstrated no alteration in the ability to kill YB cells. Furthermore, they exhibited a reduced, rather than an increased, capacity to kill certain targets such as RMA-S and C4.4-25, which are class I MHC-negative variant of RMA and EL-4, respectively. Killing of RMA-S and C4.4-25 is known to be highly dependent on the activating function of 2B4. Hence, these findings indicated that lack of EAT-2 or ERT and lack of SAP had strikingly different effects on NK functions.

### Example 3

### Decreased NK Cell Functions in Transgenic Mice Overexpressing EAT-2 or ERT, but not SAP

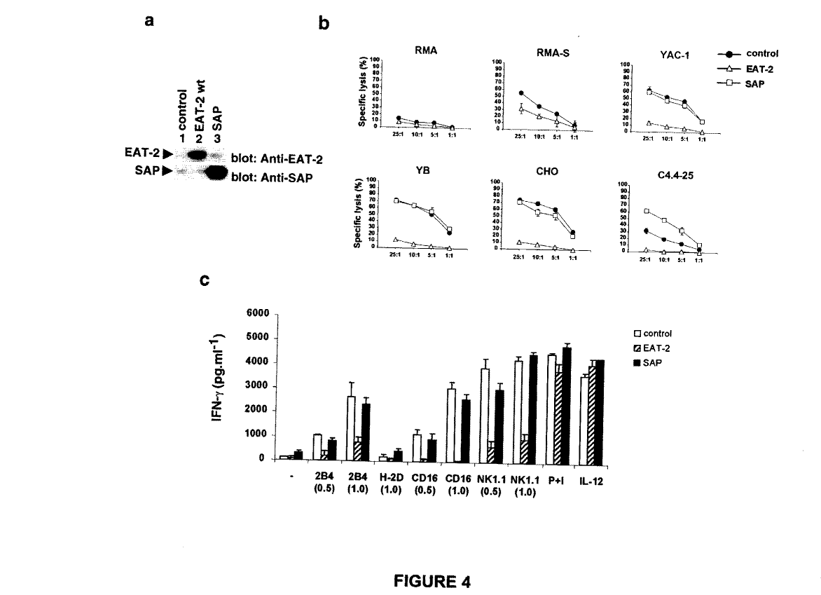

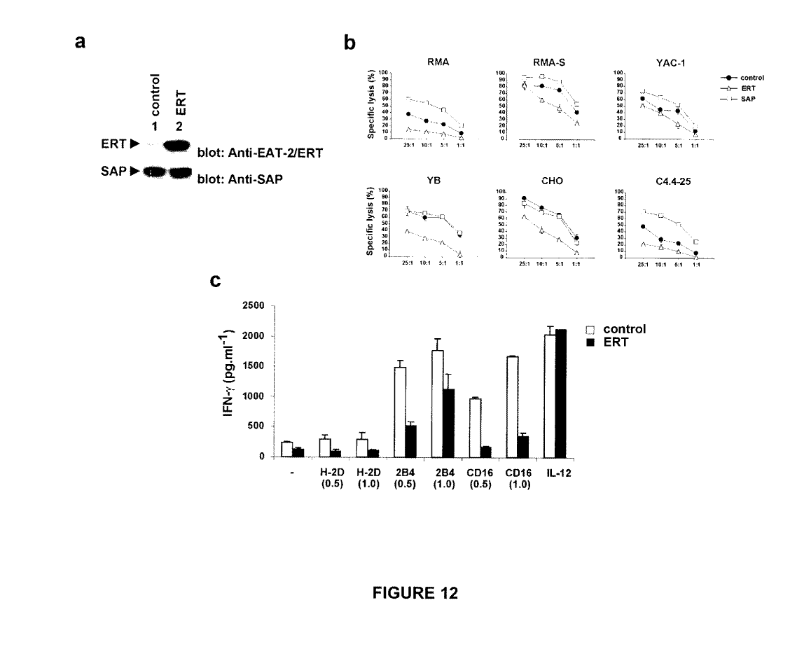

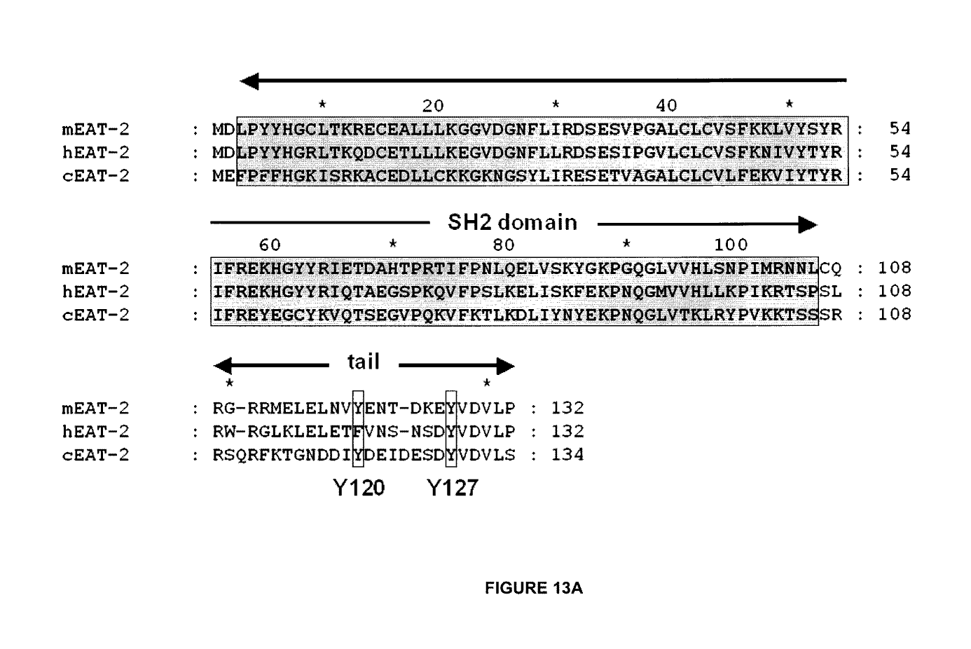

To confirm these findings, the impact of enhanced expression of EAT-2 or SAP on NK functions (FIG. 4; FIG. 12) was also compared. A transgenic mouse approach was selected for this purpose, as mouse NK cells are poor recipient for transfection or retrovirus-mediated gene transfer. cDNAs coding for EAT-2 or SAP were cloned in a CD2 promoter-driven construct, and transgenic mice were created. Overexpression of EAT-2 or SAP was confirmed by immunoblotting of cell lysates with anti-EAT-2 (FIG. 4a, first panel) or anti-SAP (second panel), respectively.

First, natural cytotoxicity was examined (FIG. 4b; FIG. 12b). As expected from the enhanced responsiveness observed in eat-2^{-/-} NK cells, augmented expression of EAT-2 suppressed the ability of NK cells to kill targets (FIG. 4b). Interestingly, this effect was broader than that observed with EAT-2 deficiency, as it concerned a wider range of targets including RMA-S, YAC-1, YB, CHO and C4.4-25. By opposition, overexpression of SAP had minimal effects on killing of most targets (FIG. 4b; FIG. 12b). The one exception was C4.4-25, which was killed much more efficiently by SAP-overexpressing NK cells. A small increase in killing of RMA. RMA-S and, occasionally, YAC-1 was also observed (FIG. 12b). In general, the effect of SAP overexpression was opposite of that of SAP deficiency (FIG. 3e; Bloch-Queyrat et al., submitted).

Next, IFN-γ production was evaluated (FIG. 4c). Enforced expression of EAT-2 caused a pronounced inhibition of IFN-γ production in response to ligation of CD16 or NKR-P1c (NK1.1), a stimulatory NK receptor expressed in C57BL/6 but not 129/Sv mice. A lesser inhibitory effect was seen in response to engagement of 2B4. By opposition, there was little or no effect on IFN-γ production in response to IL-12 or PMA plus ionomycin. Contrary to EAT-2, SAP had no significant effect on receptor-mediated IFN-γ production. This result may seem surprising considering the positive effect of SAP overexpression on killing of certain targets. One reasonable explanation is that endogenous SAP is sufficient to promote IFN-γ secretion.

Inhibitory effects analogous to those of EAT-2 were also observed in mice overexpressing ERT, although the intensity of these effects was generally less than that noted with EAT-2 (FIG. 12; data not shown).

Therefore, in combination with the data from deficient mouse strains, these results establish that EAT-2 and ERT are negative regulators of NK activity, whereas SAP is a positive regulator of NK functions.

### Example 4

### EAT-2/ERT, Like SAP, are Primarily Associated with 2B4 in NK Cells

In an attempt to explain the distinct effects of EAT-2/ERT and SAP in NK cells, two possibilities emerged. On the one hand, these divergent activities may reflect differences in the interaction with SLAM-related receptors and/or other receptors. On the other hand, they may indicate that the different SAP family adaptors are coupled to distinct downstream effectors.

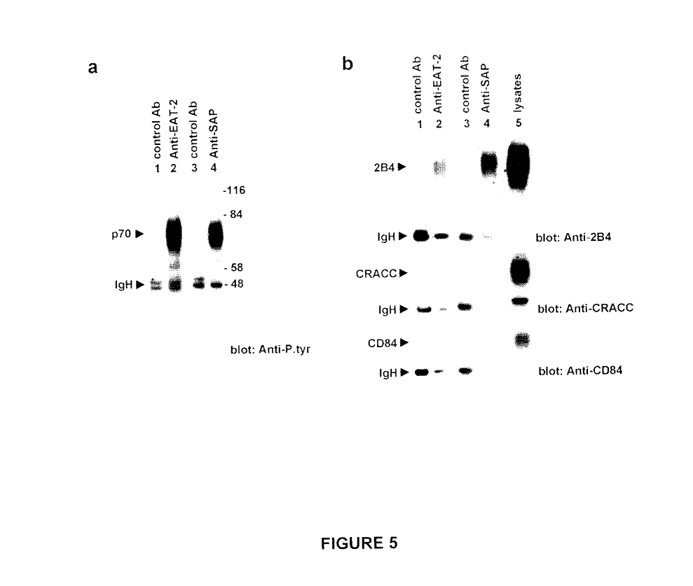

To identify the receptor(s) interacting with EAT-2, ERT and SAP in NK cells, all three molecules were immunoprecipitated from IL-2-activated mouse NK cells and probed by immunoblotting with anti-phosphotyrosine antibodies (FIG. 5a). Since our currently available immunoprecipitating antibodies cannot distinguish between EAT-2 and ERT, these two adaptors were immunoprecipitated together. This experiment revealed that EAT-2/ERT was associated with a single ˜70 kDa-tyrosine phosphorylated protein (p70) in NK cells (lane 2). A similar polypeptide was detected in anti-SAP immunoprecipitates (lane 4), but not in immunoprecipitates generated with irrelevant antibodies (lanes 1 and 3).

Mouse NK cells express three SLAM-related receptors, 2B4, CRACC and CD84, that have apparent molecular masses between 60 and 70 kDa (our unpublished results). To determine if the 70 kDa-tyrosine phosphorylated protein associated with SAP family adaptors was any of these SLAM-related receptors, immunoprecipitates of SAP-related adaptors were probed by immunoblotting with antibodies against each of these SLAM family members (FIG. 5b). As shown in FIG. 5b EAT-2/ERT (lane 2) and SAP (lane 4) were detectably associated with 2B4 (first panels), but not with CRACC (second panels) or CD84 (third panels). There was also no evidence of associations between SAP-related adaptors and other classes of stimulatory receptors including CD16 (data not shown). Thus, these findings demonstrate that EAT-2/ERT and SAP are primarily associated with 2B4 in mouse NK cells. 2B4 is constitutively tyrosine phosphorylated in mouse NK cells, seemingly due to constitutive engagement by its ligand, CD48, which is also expressed on NK cells²⁰.

### Example 5

### EAT-2/ERT, but not SAP, are Tyrosine Phosphorylated

Taking into account these results, it seemed likely that the differences in the function of EAT-2/ERT and SAP were due to coupling to distinct downstream signals. In partial support of this, it had been previously reported that, SAP, but not EAT-2 (or ERT), possessed an arginine 78-based motif in the SH2 domain that mediates binding to FynT²⁵. To ascertain whether EAT-2 and ERT might possess an alternative signalling mechanism, a careful sequence comparison of SAP-related adaptors was conducted. This analysis revealed that EAT-2 and ERT primarily differ from SAP in the carboxyl-terminal domain (FIG. 1a). While this region is highly conserved between EAT-2 and ERT (72% identity), it is completely divergent between EAT-2/ERT and SAP. In particular, the carboxyl-terminal domains of EAT-2 and ERT contain two tyrosine-based motifs, centered on tyrosine (Y) 120 and Y127, that are not present in SAP and are found in a good consensus sequences for tyrosine phosphorylation.

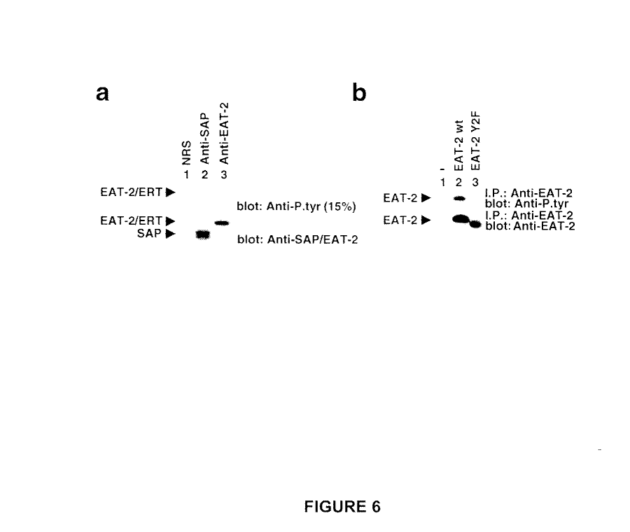

To examine if these tyrosines are phosphorylated, EAT-2/ERT was immunoprecipitated from mouse NK cells, and probed by immunoblotting with anti-phosphotyrosine antibodies (FIG. 6a, top panel). A 15 kilodalton (kDa)-tyrosine phosphorylated polypeptide was observed, consistent with EAT-2 and ERT being present in anti-EAT-2/ERT immunoprecipitates (lane 3), but not in immunoprecipitates produced with normal rabbit serum (lane 1). This polypeptide co-migrated with EAT-2/ERT (bottom panel). By opposition, there was no detectable tyrosine phosphorylation of SAP (lane 2).

To assess whether EAT-2 is phosphorylated at its carboxyl-terminal tyrosines, wild-type EAT-2 or an EAT-2 mutant in which both Y120 and Y127 were replaced by phenylalanines (“EAT-2 Y2F”) was expressed in a T-cell line in the presence of SLAM, a member of the SLAM family regulated by homotypic self-association (FIG. 6b). After cell lysis, EAT-2 was immunoprecipitated and probed by immunoblotting with anti-phosphotyrosine antibodies (top panel). This study revealed that, although wild-type EAT-2 (lane 2) was tyrosine phosphorylated, EAT-2 Y2F (lane 3) was not phosphorylated. This finding supports the idea that EAT-2 is phosphorylated at one or both of its carboxyl-terminal tyrosines.

### Example 6

### The Carboxyl-Terminal Tyrosines of EAT-2 are Required for Inhibition of NK Functions

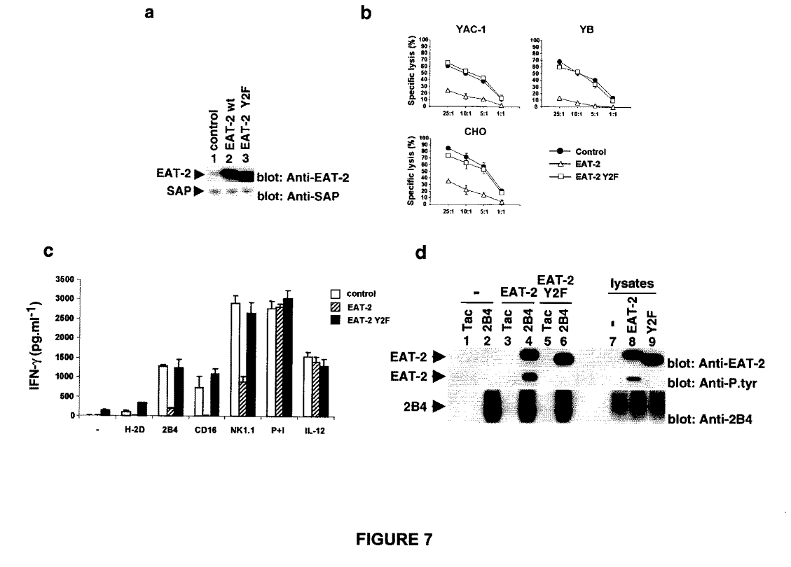

Given these findings, it was next determined whether the carboxyl-terminal tyrosines were required for the inhibitory function of EAT-2 in NK cells. To this end, transgenic mice expressing EAT-2 Y2F in NK cells were generated as detailed above. Immunoblot analyses demonstrated that the levels of EAT-2 present in NK cells from these animals were comparable to those seen in NK cells overexpressing wild-type EAT-2 (FIG. 7a, top panel). Despite this, and unlike wild-type EAT-2, the EAT-2 Y2F mutant did not inhibit natural cytotoxicity towards YAC-1, YB and CHO cells (FIG. 7b). Likewise, it failed to suppress IFN-γ secretion in response to 2B4, CDI6 or NKR-P1c ligation (FIG. 7c).

It was important to ensure that mutation of the carboxyl-terminal tyrosines did not interfere with the ability of EAT-2 to associate with 2B4 (FIG. 7d). For this purpose, Cos-1 cells were transfected with cDNAs encoding wild-type EAT-2 or EAT-2 Y2F, in the presence of 2B4 and of an activated version of FynT to enable 2B4 tyrosine phosphorylation. Following cell lysis, 2B4 was immunoprecipitated and probed by immunoblotting with anti-EAT-2 antibodies (first panel). This analysis showed that EAT-2 Y2F (lane 6) was associated with 2B4 to the same extent as wild-type EAT-2 (lane 4). These results imply that the carboxyl-terminal tyrosines of EAT-2 are dispensable for the interaction between EAT-2 and 2B4 but are required for EAT-2 activity in NK cells

### Example 7

### EAT-2 Inhibits Proximal Events in NK Receptor Signalling

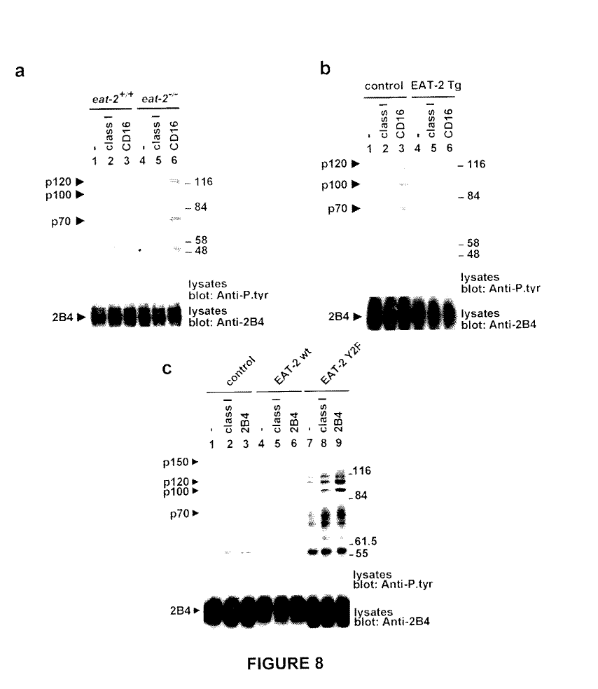

A careful examination of the receptors inhibited by EAT-2, e.g., CD16, Ly49D, NKG2D, NKR-P1c and 2B4, indicated that all these receptors function by inducing protein tyrosine phosphorylation¹⁻³. Considering this observation, the effects of EAT-2 on intracellular protein tyrosine phosphorylation were examined (FIGS. 8a and b). First, eat-2^{+/+} and eat-2^{-/-} NK cells were stimulated with anti-CD16 antibodies and lysates were probed by immunoblotting with anti-phosphotyrosine antibodies (FIG. 8a, top panel). Compared to eat-2^{+/+} NK cells (lanes 1-3), EAT-2-deficient NK cells (lanes 4-6) exhibited an increase in CD16-induced protein tyrosine phosphorylation. Most CD16-regulated substrates were affected by this augmentation. Such a finding suggested that EAT-2 was inhibiting proximal events in NK signalling. To validate this conclusion, protein tyrosine phosphorylation was also examined in NK cells overexpressing EAT-2 (FIG. 8b). It was observed that basal protein tyrosine phosphorylation was reduced in EAT-2-overexpressing NK cells (lane 4). Moreover, CD16-induced protein tyrosine phosphorylation was completely abrogated (compare lane 3 and lane 6) in such cells. A similar inhibitory effect was seen in cells stimulated via 2B4 (FIG. 8c).

Finally, it was determined whether the inhibition of protein tyrosine phosphorylation was dependent on the carboxyl-terminal tyrosines of EAT-2 (FIG. 8c). The results of this experiment showed that, contrary to wild-type EAT-2 (lanes 4-6), the EAT-2 Y2F mutant (lanes 7-9) did not suppress baseline or 2B4-induced protein tyrosine phosphorylation. In fact, it caused an increase in overall protein tyrosine phosphorylation, suggesting that it had a dominant-negative effect. However, this effect would only seem to be partial, as EAT-2 Y2F did not cause any enhancement of natural cytotoxicity or IFN-γ release (see FIG. 7).

### Example 8

### Inhibition of NK Cell Function by Human EAT-2

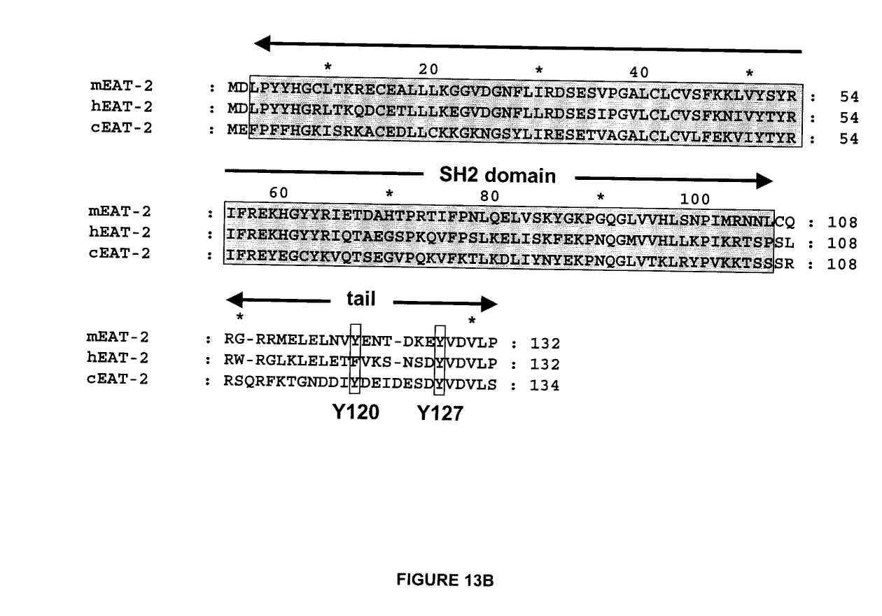

Because of the high sequence conservation between mice and human EAT-2 (FIGS. 13A and B), it is likely that they will share the same biological functions. More specifically, the conservation of the SH2 domain as well as the tyrosines in the carboxyl-terminal tail of EAT-2, in particular tyrosine 127, implies that the mechanism of EAT-2-mediated inhibition of NK functions is evolutionarily conserved and that it also exists in humans.

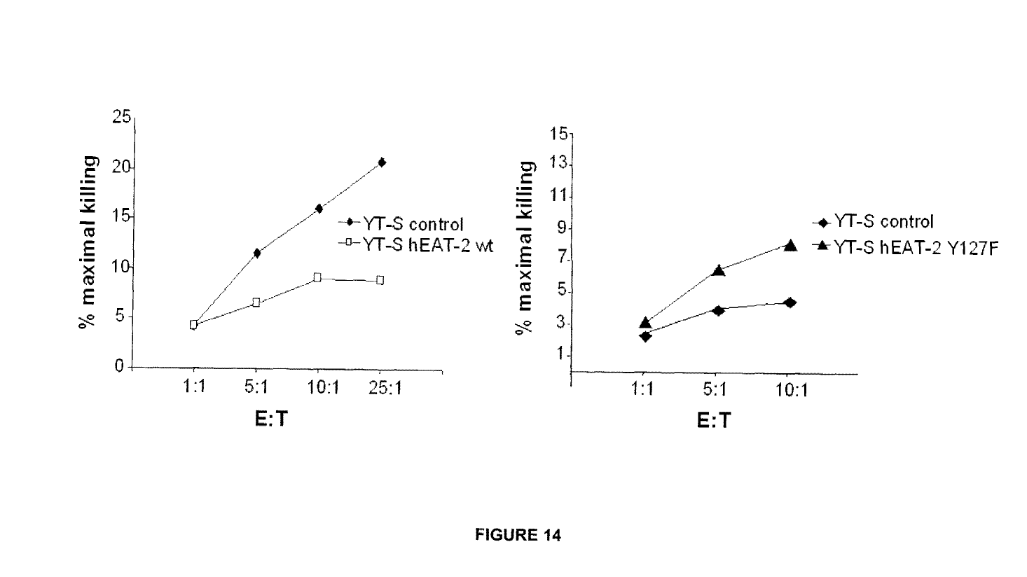

To validate this hypothesis, the inhibition of natural killing by human EAT-2 was evaluated. Wild-type human EAT-2 or human EAT-2 mutated at tyrosine 127 (EAT-2 Y127F—where the tyrosine at position 127 is replaced by a phenylalanine) was overexpressed in the human NK cell line YT-S, Natural killing towards K562 was measured using a ⁵¹Cr release assay (FIG. 14). These results show that human EAT-2 is also an inhibitor of human NK cell functions. Furthermore, they reveal that this inhibition is mediated by a mechanism that is strictly dependent on the carboxyl-terminal tyrosine (tyrosine 127) of human EAT-2. Thus, from these results it can be concluded that human and mice EAT-2 share similar, if not identical cellular functions, thereby validating the mice models of the present invention as model system for humans.

### Example 9

### EAT-2 and ERT as Therapeutic Targets to Modulate Innate Immune Cells Functions

In this application, the inventors sought to determine the role of the SAP-related adaptor EAT-2 in immune cells. While analyzing the sequence of eat-2 to generate an EAT-2-deficient mouse, an additional sap-related gene in the mouse genome was uncovered. This gene, named ert, is located within 26 kb of eat-2 on mouse chromosome 1. Given the close physical proximity of ert and eat-2 in the genome, it is likely that the two genes were generated by duplication of a common ancestor. ert encodes a protein very similar to EAT-2 (82% identity) and is expressed exclusively in NK cells. In contrast to the ert gene, eat-2 is expressed in NK cells and other immune cells such as DCs and macrophages, while sap is expressed in NK cells and T-cells. From these expression patterns, it is noticeable that NK cells contain all three known SAP-related adaptors.

Thus, eat-2^{-/-} mice and ert^{-/-} mice were created, and the impact of these mutations on NK functions was compared to that of SAP deficiency. As is the case for lack of SAP expression (Bloch-Queyrat et al., submitted), a deficiency in EAT-2 or ERT had no appreciable impact on NK development. However, eat-2^{-/-} mice and ert^{-/-} mice exhibited striking abnormalities in NK functions. NK cells from both mice demonstrated an increase in the ability of NK receptors such as CD16, NKG2D, Ly49D and 2B4 to trigger IFN-γ production. Likewise, EAT-2-deficient NK cells had an enhanced ability to kill targets like YB and CHO, while ERT-deficient NK cells had an increased cytotoxic response towards YB. NK-mediated killing of YB and CHO is highly dependent on the activation of the NK receptor Ly49D, which recognizes class I MHC on targets. The impact of EAT-2 or ERT deficiency on NK functions was specific, since sap⁻ NK cells were less efficient, instead of more efficient, at killing target cells (as shown herein). Moreover, they exhibited a decrease, rather than an increase, in 2B4-triggered IFN-γ release (Block-Queyrat et al., submitted).

To validate these observations, the influence of enforced expression of EAT-2, ERT or SAP on NK functions was also studied. NK cells from transgenic mice overexpressing EAT-2 or ERT showed that both adaptors suppressed the ability of NK cells to produce IFN-γ in response to CD16, NKG2D, Ly49D, NKR-P1c and 2B4. Likewise, they inhibited the capacity of NK cells to kill targets. Hence, the effects triggered by overexpression of EAT-2 or ERT were a mirror image of those caused by deficiency of these adaptors. Of note, however, augmented expression of EAT-2 or ERT impacted on a broader range of targets. It is possible that this difference resulted from partial compensation by the remaining adaptor in NK cells lacking EAT-2 or ERT. In contrast to EAT-2 and ERT, enforced expression of SAP had no effect on IFN-γ secretion. Moreover, it augmented, rather than decreased, the ability of NK cells to kill target cells, seemingly in a 2B4-dependent fashion. Considering these findings, it can be concluded that EAT-2 and ERT inhibit the function of several stimulatory NK receptors. In contrast, SAP is principally implicated in promoting the activity of 2B4.

What explains the functional differences between EAT-2/ERT and SAP in NK cells? It seems unlikely that these differences reflect a divergence in the type of receptors interacting with the adaptors. This idea is in keeping with the observation that the SH2 domain of EAT-2 and SAP could associate with the same spectrum of SLAM family receptors in yeast cells²². Moreover, it is consistent with the finding that both EAT-2/ERT and SAP exclusively interacted with 2B4 in mouse NK cells (as shown herein). None of these adaptors was detectably associated with the other members of the SLAM family expressed in NK cells, i.e. CRACC and CD84, or with other classes of NK receptors. On this basis, it is argued that EAT-2/ERT and SAP are probably recruited to the membrane by a similar mechanism, which involves binding of their SH2 domain to tyrosine phosphorylated 2B4.

The distinct roles of EAT-2/ERT and SAP in NK cells are likely caused by differences in their intrinsic signalling mechanism. In agreement with this, it was published elsewhere by the laboratory of the inventors that SAP, but not EAT-2, is able to recruit the Src-related kinase FynT by way of a second binding surface centered on R78 in its SH2 domain²⁵. This activity is critical for the capacity of SAP to promote the activating function of 2B4²⁰. In contrast, it was noted herein that EAT-2 and ERT, but not SAP, possess two conserved tyrosines in their carboxyl-terminal tail, Y120 and Y127. Further studies revealed that EAT-2/ERT, but not SAP, were tyrosine phosphorylated in ex vivo mouse NK cells, and that mutation of these two tyrosines (“Y2F” mutation) abolished tyrosine phosphorylation of EAT-2. Moreover, the Y2F mutation eliminated the capacity of EAT-2 to inhibit NK functions. Consequently, it is postulated that the inhibitory effect of EAT-2 and ERT on NK functions is caused by a unique signalling mechanism implicating tyrosine phosphorylation of their tail.

The purpose of expressing different SAP-related adaptors in NK cells is being unravelled. Based on the available evidence, it seems likely that the relative expression of these molecules helps in setting the threshold of NK responsiveness. A predominance of EAT-2/ERT over SAP probably suppresses NK responsiveness, while a preponderance of SAP over EAT-2/ERT likely facilitates NK reactivity, at least during 2B4-dependent responses. This model is consistent with the earlier finding that, whereas expression of EAT-2 is relatively constant in immature and mature NK cells, SAP is most abundantly expressed in mature NK cells¹². Thus, a preponderant expression of EAT-2 may aid in suppressing the reactivity of immature NK cells, while augmented SAP expression may assist in enhancing the responsiveness of mature NK cells.

EAT-2 and ERT may be especially involved in a phenomenon referred to as “non-MHC-dependent NK inhibition”. Indeed, whereas most mature NK cells express class I MHC-specific inhibitory receptors such as inhibitory Ly49s and KIRs that prevent inappropriate NK reactivity, some NK populations, including immature NK cells and a subset of mature NK cells (˜10% in C57BL/6 mice), express little or no self-class I MHC-reactive inhibitory receptors^{8,26,27}. In spite of this, these cells are self-tolerant and do not mediate autologous killing. These observations imply that other inhibitory mechanisms repress their function. Although the identity of these mechanisms remains to be formally established, it has been proposed that receptors such as 2B4 may play an important role in this process. In this light, it is attractive to hypothesize that EAT-2 and ERT, as a result of their association with 2B4, participate in non-MHC-dependent NK inhibition. Analyses of NK self-reactivity in mice lacking EAT-2 or ERT will aid in assessing this possibility.

The results presented herein, together with earlier findings, also provide a plausible explanation for the ability of 2B4 to mediate stimulatory or inhibitory signals in NK cells. As revealed by analyses of SAP-deficient human and mouse NK cells¹⁶⁻²⁰ (Bloch-Queyrat et al., submitted), the capacity of 2B4 to enhance NK cell-mediated cytotoxicity and IFN-γ secretion seems to be dependent on SAP. This notion is also supported by the present results relating to SAP-overexpressing transgenic NK cells, which exhibited enhanced killing of C4.4-25 cells, a 2B4-dependent target (this report). By opposition, this inhibitory activity of 2B4 may be mediated by EAT-2 and ERT. This model could explain why 2B4 is especially inhibitory in immature NK cells, which express little or no SAP but express EAT-2, or in NK cells derived from SAP-deficient humans and mice^{17,26} (Bloch-Queyrat et al., submitted). Nonetheless, other mechanisms may also contribute to 2B4-mediated inhibition. Along these lines, it was recently reported that, in the absence of SAP and EAT-2, 2B4 can associate in vitro with other negative regulators of immune cell activation such as the phosphatases SHP-1, SHP-2, SHIP, and the inhibitory PTK Csk²⁸.

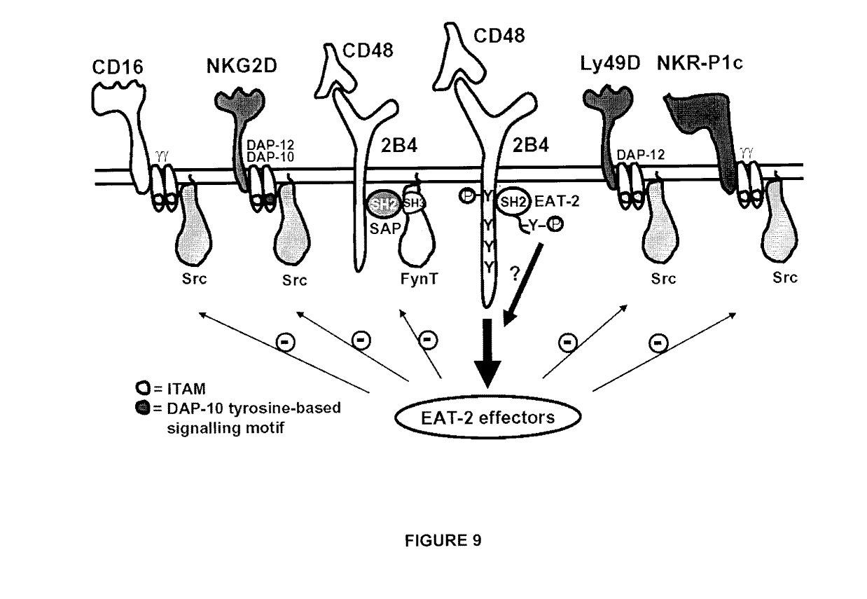

The stimulatory NK receptors found to be inhibited by EAT-2 and ERT are either expected or known to utilize Src family kinases in their signalling mechanism (FIG. 9)¹⁻³. This is the case of CD16, Ly49D, NKG2D and NKR-P1c, which are associated with ITAM-containing sub-units that necessitate Src-related PTKs for their tyrosine phosphorylation. It is also the situation of 2B4, which utilizes SAP and associated FynT for positive signalling²⁰. In comparison, EAT-2 and ERT had little or no effect on other classes of receptors. These included the IL-12 receptor, a cytokine receptor using Jak kinases, and the IL-18 receptor, a chemokine receptor triggering G protein-coupled pathways. Likewise, EAT-2 and ERT had no influence on the responsiveness to PMA plus ionomycin, implying that they repress steps upstream of protein kinase C activation and calcium fluxes. Such an idea is also supported by the finding that EAT-2 inhibited the most proximal signal triggered by engagement of CD16 or 2B4, that is, protein tyrosine phosphorylation. Thus, it is proposed that EAT-2 and ERT inhibit the function of stimulatory NK receptors by uncoupling these receptors from Src kinase-dependent protein tyrosine phosphorylation. Whereas the precise molecular effectors responsible for this inhibition remain to be characterized, candidates include Csk, SHP-1, ubiquitin ligases like c-Cbl or a combination of these factors.

Although eat-2 and ert are both operational in mice, only EAT-2 seems to be functional in humans (inventors' unpublished results). The human ERT gene has seemingly evolved into a non-coding pseudo-gene. Given this, it is likely that the combined function of EAT-2 and ERT in mice is served by EAT-2 alone in humans. In view of the high conservation between EAT-2 and ERT which probably have partially redundant functions in the mouse, the generation of mice lacking either adaptor alone is unlikely to estimate fully the functional importance of EAT-2 in humans.

### Example 9

### Material and Methods

### Cells

Mouse thymocytes, splenic T-cells, splenic B-cells, peritoneal macrophages and bone marrow-derived mast cells were obtained from C57BL/6 mice (Harlan, Chicago, Ill.) according to standard protocols^{29,30}. Purity was verified by flow cytometry and confirmed to be greater than 90% in all cases. Mouse dendritic cells were derived from bone marrow cells cultured in RPMI 1640 medium supplemented with 10% fetal bovine serum (FBS), antibiotics, β-mercaptoethanol (β-ME) and granulocyte-macrophage colony-stimulating factor (GM-CSF), according to an established protocol³¹. After 9 days, over 90% of cells were CD11b⁺/CD11c⁺ (data not shown). Cells were then treated or not for 24 hours with lipopolysaccharide (1 μg.ml⁻¹; Sigma-Aldrich Canada Ltd., Oakville, Ontario, Canada). This stimulation resulted in DC activation, as assessed by up-regulation of class II MHC, CD40 and CD86 (data not shown). Mouse NK cells were isolated from spleen by negative selection, using an NK cell isolation cocktail (Stem Cell Technologies, Vancouver, BC, Canada). ˜90% of purified cells were NK1.1⁺ and CD3⁻ (data not shown). Cells were then propagated in RPMI 1640 medium supplemented with 10% FBS, antibiotics, β-ME and IL-2 (1000 U/ml), and were used for experimentation after 7 days. At that time, greater than 95% of cells were NK1.1⁺, 2B4⁺, DX-5⁺ and CD3⁻ (data not shown). YAC-1 (H-2^{a}), RMA (H-2^{b}), RMA/S (H-2^{b}-negative), EL-4 (H-2^{b}), C4.4-25 (H-2^{b}-negative), CHO and YB cell lines were grown in RPMI-1640 supplemented with 10% FCS and antibiotics. BI-141 is an antigen-specific mouse T-cell line that does not express SLAM, SAP, EAT-2 or ERT (data not shown). Derivatives expressing wild-type EAT-2 or EAT-2 Y120,127F (“Y2F”) were generated by retroviral infection according to standard protocols^{20,32,33}.

### DNAs Constructs and Site-Directed Mutagenesis

cDNAs encoding mouse EAT-2, 2B4 and SH2 domain-deleted FynT (ΔSH2 FynT) were reported elsewhere^{20,25,34-36}. The cDNA coding for mouse ERT was kindly obtained from RIKEN, Japan. Point mutations were introduced in eat-2 using the QuickChange™ Site-Directed Mutagenesis Kit (Stratagene, LaJolla, Calif.). All cDNAs were fully sequenced to ensure that they carried no unwanted mutations (data not shown).

### Mice Lacking EAT-2, ERT or SAP

Mice lacking EAT-2 or ERT were created by a similar strategy. In brief, the eat-2 and ert genes were amplified by PCR from a BAC clone derived from 129/Sv mice, and 5′ and 3′ genomic fragments were cloned on either side of the neo gene in the vector pGT-N28 (New England Biolabs, Beverley, Mass.). These constructs disrupt exon 2 of the eat-2 and ert genes. Based on data obtained by others for the sap gene, this approach is expected to produce null alleles³⁸. After linearizing with NotI, the DNAs were electroporated into the ES cell line R1, and transfected cells were selected with G418. Individual clones were screened by PCR and Southern blotting, and positive clones were injected into blastocysts. Mice with an appropriately recombined eat-2 or ert gene were bred to 129/Sv mice. In subsequent generations, mice carrying the eat-2^{-/-} or ert^{-/-} mutation were identified by PCR amplification of genomic DNA. Wild-type littermates were used as controls. For both eat-2^{-/-} mice and ert^{-/-} mice, analogous results were obtained with mice derived from two independent ES clones (data not shown). SAP-deficient mice (sap⁻) were described elsewhere³⁸.

### Transgenic Mice

To produce EAT-2 or ERT transgenic mice, full-length mouse cDNAs coding for wild-type EAT-2, EAT-2 Y2F or wild-type ERT were inserted in a CD2 promoter-driven construct (provided by Dr. Dimitri Kioussis, London, England). Transgenic mice were produced in the mixed C57BL/6-C3H background by the IRCM Transgenic Facility, and were back-crossed for at least 6 generations to the C57BL/6 background. Mice expressing the sap transgene were reported elsewhere²⁵. Progenies from at least two independent founders for each type of transgenic mouse were analyzed (data not shown).

### Antibodies

Rabbit antibodies directed against 2B4, SAP, FynT and phosphotyrosine were described in other publications^{20,33-36,39-41}. Polyclonal rabbit antisera against CRACC and CD84 will be reported elsewhere (applicant's unpublished results). Polyclonal antibodies against EAT-2 were generated in rabbits using a TrpE fusion protein encompassing the carboxyl-terminal tail of EAT-2 or a GST fusion construct bearing the SH2 domain of EAT-2. These antibodies reacted against EAT-2 and ERT, but not SAP (data not shown). Monoclonal antibodies (MAbs) recognizing EAT-2 (MAbs 15G3 or 5A7) were generated in rats, using a GST fusion protein containing the full-length sequence of EAT-2. MAb 15G3 reacts against EAT-2 and ERT, but not SAP, whereas MAb 5A7 reacts with EAT-2, but not ERT or SAP (data not shown). Rat MAbs against SAP will be described elsewhere. MAbs against 2B4 (MAb 2B4), CD16 (MAb 2.4G2), class I MHC (MAb KH95), Ly49D (MAb 4E5), NKG2D (MAb CX5) and NKR-P1c (MAb PK136) were purchased from eBioscience (San Diego, Calif.) or BD Biosciences (Mississauga, Ontario, Canada).

### NK Cell Assays

IL-2-activated NK cells were stimulated in triplicate for 20 hours with the indicated concentrations of antibodies coated on plastic, phorbol myristate acetate (PMA; 50 ng.ml⁻¹) plus ionomycin (1 μg.ml⁻¹), IL-12 (5 ng.ml⁻¹) or IL-18 (20 ng.ml⁻¹). Supernatants were subsequently harvested and assayed for IFN-γ production by ELISA, according to the protocol outlined by the manufacturer (R&D Systems, Minneapolis, Minn.). Natural cytotoxicity was evaluated using a standard ⁵¹Cr release assay. Briefly, NK cells were incubated for 4 hours at 37° C. with ⁵¹Cr-labeled target cells (5×10³), at the indicated effector: target ratios. The release of ⁵¹Cr in the supernatant was measured using a gamma counter. The percentage of specific lysis was calculated according to the following formula: (experimental-spontaneous release)/(maximum-spontaneous release)×100. All assays were done in duplicate.

### Transfections

Cos-1 cells were transfected with the indicated cDNAs using Lipofectamine™ Plus (Invitrogen, Burlington, Ontario, Canada).

### Immunoprecipitations and Immunoblots

Immunoprecipitations and immunoblots were performed as described earlier^{39,42}. Immunoreactive products were detected using either ¹²⁵I-protein A, horseradish peroxidase (HRP)-coupled protein A, ¹²⁵I-rabbit anti-mouse IgG, HRP-sheep anti-mouse (SAM) IgG or HRP-goat anti-rat (GAR) IgG. All secondary reagents were purchased from Amersham Biosciences, Baie d'Urfé, Quebec, Canada.

RT-PCRs

RNA was isolated from cells using Rneasy™ columns (Qiagen, Mississauga, Ontario, Canada). Integrity was confirmed by migrating aliquots in agarose gels and staining with ethidium bromide (data not shown). RT-PCRs were conducted with the Qiagen OneStep™ RT-PCR kit, according to the protocol outlined by the manufacturer. Protocols were optimized to ensure that the reactions were linear and specific (data not shown). The following oligonucleotides were used: eat-2: 5′-TGATGCTCATACTCCAAGAACG-3′ and 5′-AGGCAAGACGTCCACATACTC-3′; ert: 5′-AGGATAGAGACTGAGCCCAG-3′ and 5′-AGGCAAGACGTCCACATACTC-3′; sap: 5′-ACAGAAACAGGTTCTTGGAGTG3′ and 5′GCATTCAGGCAGATATCAGAATC3′; gapdh: 5′-GGGTGGAGCCAAACGGGTC-3′ and 5′-GGAGTTGCTGTTGAAGTCGCA-3′. PCR products were resolved in polyacrylamide or agarose gels, and detected by ethidium bromide staining.

In summary, the present invention relates to the identification of EAT-2 and ERT as novel therapeutic targets for the modulation of innate immune cells functions. It was determined that EAT-2 and its close relative ERT are novel negative regulators of NK cell reactivity. Since EAT-2 is also expressed in other innate immune cells like DCs and macrophages, it is likely that it provides an analogous function in these cell types as well. Lastly, in the light of the results presented herein, it is hypothesized that, as for SAP, mutations or altered expression of EAT-2 are implicated in human immune dysfunctions. Given that EAT-2 is a negative regulator of NK activation, these alterations might lead to NK cell self-reactivity and auto-immunity.

Although the present invention has been described hereinabove by way of specific embodiments thereof, it can be modified, without departing from the spirit and nature of the subject invention as defined in the appended claims.

### REFERENCES

1. Lanier, L. L. NK Cell Recognition. Annu. Rev. Immunol. 23, 225-74 (2005).
2. Raulet, D. H. Roles of the NKG2D immunoreceptor and its ligands. Nat. Rev. Immunol. 3, 781-790 (2003).
3. Vivier, E., Nunes, J. A., & Vely, F. Natural killer cell signaling pathways. Science 306, 1517-1519 (2004).
4. Mathew, P. A. et al. Cloning and characterization of the 2B4 gene encoding a molecule associated with non-MHC-restricted killing mediated by activated natural killer cells and T cells. J. Immunol. 151, 5328-5337 (1993).
5. Valiante, N. M. & Trinchieri, G. Identification of a novel signal transduction surface molecule on human cytotoxic lymphocytes. J. Exp. Med. 178, 1397-1406 (1993).
6. Veillette, A. & Latour, S. The SLAM family of immune-cell receptors. Curr. Opin. Immunol. 15, 277-285 (2003).
7. Engel, P., Eck, M. J., & Terhorst, C. The SAP and SLAM families in immune responses and X-linked lymphoproliferative disease. Nat. Rev. Immunol. 3, 813-821 (2003).
8. Kumar, V. & McNerney, M. E. A new self: MHC-class-1-independent Natural-killer-cell self-tolerance. Nat. Rev. Immunol. (2005) (in press).
9. Brown, M. H. et al. 2B4, the natural killer and T cell immunoglobulin superfamily surface protein, is a ligand for CD48. J. Exp. Med. 188, 2083-2090 (1998).
10. Latchman, Y., McKay, P. F., & Reiser, H. Identification of the 2B4 molecule as a counter-receptor for CD48. J Immunol. 161, 5809-5812 (1998).
11. Veillette, A. SLAM Family Receptors Regulate Immunity with and without SAP-related Adaptors. J. Exp. Med. 199, 1175-1178 (2004).
12. Lee, K. M. et al. 2B4 Acts As a Non-Major Histocompatibility Complex Binding Inhibitory Receptor on Mouse Natural Killer Cells. J Exp Med 199, 1245-1254 (2004).
13. Vaidya, S. V. et al. Targeted Disruption of the 2B4 Gene in Mice Reveals an In Vivo Role of 2B4 (CD244) in the Rejection of B16 Melanoma Cells. J Immunol. 174, 800-807 (2005).
14. Nichols, K. E. et al. Regulation of NKT cell development by SAP, the protein defective in XLP. Nat. Med. 11, 340-345 (2005).
15. Latour, S. & Veillette, A. The SAP family of adaptors in immune regulation. Semin. Immunol. 16, 409-419 (2004).
16. Nakajima, H. et al. Patients with X-linked lymphoproliferative disease have a defect in 2B4 receptor-mediated NK cell cytotoxicity. Eur. J Immunol. 30, 3309-3318 (2000).
17. Parolini, S. et al. X-linked lymphoproliferative disease. 2B4 molecules displaying inhibitory rather than activating function are responsible for the inability of natural killer cells to kill Epstein-Barr virus-infected cells. J Exp. Med. 192, 337-346 (2000).
18. Tangye, S. G., Phillips, J. H., Lanier, L. L., & Nichols, K. E. Functional requirement for SAP in 2B4-mediated activation of human natural killer cells as revealed by the X-linked lymphoproliferative syndrome. J Immunol. 165, 2932-2936 (2000).
19. Benoit, L., Wang, X., Pabst, H. F., Dutz, J., & Tan, R. Defective NK cell activation in X-linked lymphoproliferative disease. J Immunol. 165, 3549-3553 (2000).
20. Chen, R. et al. Molecular Dissection of 2B4 Signaling: Implications for Signal Transduction by SLAM-Related Receptors. Mol. Cell Biol. 24, 5144-5156 (2004).
21. Thompson, A. D. et al. EAT-2 is a novel SH2 domain containing protein that is up regulated by Ewing's sarcoma EWS/FLI1 fusion gene. Oncogene 13, 2649-2658 (1996).
22. Morra, M. et al. Structural basis for the interaction of the free SH2 domain EAT-2 with SLAM receptors in hematopoietic cells. EMBO J 20, 5840-5852 (2001).
23. Nichols, K. E. et al., Inactivating mutations in an SH2 domain-encoding gene in X-linked lymphoproliferative syndrome. Proc Natl Acad Sci USA 95, 13765-13770 (1998).
24. Coffey, A. J. et al. Host response to EBV infection in X-linked lymphoproliferative disease results from mutations in an SH2-domain encoding gene. Nat. Genet. 20, 129-135 (1998).
25. Latour, S. et al. Binding of SAP SH2 domain to FynT SH3 domain reveals a novel mechanism of receptor signalling in immune regulation. Nat. Cell Biol. 5, 149-154 (2003).
26. Sivori, S. et al. Early expression of triggering receptors and regulatory role of 2B4 in human natural killer cell precursors undergoing in vitro differentiation. Proc. Natl. Acad. Sci. U.S.A 99, 4526-4531 (2002).
27. Fernandez, N. C. et al. A subset of natural killer cells achieve self-tolerance without expressing inhibitory receptors specific for self MHC molecules. Blood (2005) (in press).
28. Eissmann, P. et al. Molecular basis for positive and negative signaling by the natural killer cell receptor 2B4 (CD244). Blood (2005) (in press).
29. Veillette, A., Thibaudeau, E., & Latour, S. High expression of inhibitory receptor SHPS-1 and its association with protein-tyrosine phosphatase SHP-1 in macrophages. J Biol Chem 273, 22719-22728 (1998).
30. Davidson, D. & Veillette, A. PTP-PEST, a scaffold protein tyrosine phosphatase, negatively regulates lymphocyte activation by targeting a unique set of substrates. EMBO J 20, 3414-3426 (2001).
31. Lutz, M. B. et al. An advanced culture method for generating large quantities of highly pure dendritic cells from mouse bone marrow. J Immunol. Methods 223, 77-92 (1999).
32. Latour, S., Fournel, M., & Veillette, A. Regulation of T-cell antigen receptor signalling by Syk tyrosine protein kinase. Mol Cell Biol 17, 4434-4441 (1997).
33. Chen, R. et al. Molecular Dissection of 2B4 Signaling: Implications for Signal Transduction by SLAM-Related Receptors. Mol. Cell Biol. 24, 5144-5156 (2004).
34. Latour, S. et al. Regulation of SLAM-mediated signal transduction by SAP, the X-linked lymphoproliferative gene product. Nat Immunol 2, 681-690 (2001).
35. Chow, L. M., Fournel, M., Davidson, D., & Veillette, A. Negative regulation of T-cell receptor signalling by tyrosine protein kinase p50csk. Nature 365, 156-160 (1993).
36. Lemay, S., Davidson, D., Latour, S., & Veillette, A. Dok-3, a novel adapter molecule involved in the negative regulation of immunoreceptor signaling. Mol. Cell Biol. 20, 2743-2754 (2000).
37. Stein, P. L., Lee, H. M., Rich, S., & Soriano, P. pp 59fyn mutant mice display differential signaling in thymocytes and peripheral T cells. Cell 70, 741-750 (1992).
38. Yin, L. et al. Mice deficient in the X-linked lymphoproliferative disease gene sap exhibit increased susceptibility to murine gammaherpesvirus-68 and hypo-gammaglobulinemia. J. Med. Virol. 71, 446-455 (2003).
39. Davidson, D., Chow, L. M., Fournel, M., & Veillette, A. Differential regulation of T cell antigen responsiveness by isoforms of the src-related tyrosine protein kinase p59fyn. J Exp Med 175, 1483-1492 (1992).
40. Fournel, M., Davidson, D., Weil, R., & Veillette, A. Association of tyrosine protein kinase Zap-70 with the protooncogene product p120c-cbl in T lymphocytes. J Exp Med 183, 301-306 (1996).
41. Davidson, D., Chow, L. M., Fournel, M., & Veillette, A. Differential regulation of T cell antigen responsiveness by isoforms of the src-related tyrosine protein kinase p59fyn. J Exp Med 175, 1483-1492 (1992).
42. Veillette, A., Bookman, M. A., Horak, E. M., & Bolen, J. B. The CD4 and CD8 T cell surface antigens are associated with the internal membrane tyrosine-protein kinase p56lck. Cell 55, 301-308 (1988).

1.-5. (canceled)

6. A method to identify potentially therapeutic agents which modulate EAT-2 activity useful for the treatment and prevention of a disease, comprising:
i) contacting said agent with cells expressing EAT-2; and
ii) assessing said cells for an alteration in a EAT-2 biological activity, said biological activity being related to innate immune cell function;
wherein a potentially therapeutic agent useful for the treatment of a disease is identified when said biological activity related to innate immune cell function is modulated in the presence of a candidate agent as compared to in the absence thereof.

7. The method of claim 6, wherein the modulation is an inhibition and the disease is selected from an infectious disease and cancer.

8. The method of claim 6, wherein the modulation is an increase in EAT-2 activity and the disease is an autoimmune disease.

9. The method of claim 6, wherein the biological activity is a production of IFN-γ and a capacity to kill a target cell.

10.-11. (canceled)

12. A short interfering RNA (siRNA) molecule, useful for the treatment of cancer and infectious disease, that decreases the expression of EAT-2 gene by RNA interference comprising a sense region and an antisense region, wherein said antisense region comprises a sequence complementary to a EAT-2 RNA sequence and the sense region comprises a sequence complementary to the antisense of said EAT-2 RNA sequence, and wherein the sense region of said siRNA is at least 80% identical to a portion of EAT-2 as set forth in SEQ ID NO:7 or 8.

13. The siRNA of claim 12, wherein said siRNA molecule is assembled from two nucleic acid fragments, wherein one fragment comprises the sense region and the second fragment comprises the antisense region of said siRNA molecule.

14. The siRNA of claim 13, wherein said sense region and said antisense region are covalently connected via a linker molecule.

15. The siRNA of claim 14, wherein said linker molecule is a polynucleotide linker molecule.

16. The siRNA molecule of claim 12, wherein said sense region comprises a 3′-terminal overhang of 1 to 5 nucleotides in length and said antisense region comprises a 3′-terminal overhang of 1 to 5 nucleotides in length.

17. The siRNA molecule of claim 12, wherein said sense and antisense regions comprise at least one nucleotide that is chemically modified in at least one of sugar, base, or backbone moiety.

18. The siRNA molecule of claim 12, comprising a double stranded region of about 10 to 28 nucleotides in length.

19. The siRNA molecule of claim 18, wherein said siRNA molecule is linked to at least one receptor binding ligand.

20. (canceled)

21. A method of enhancing natural killer (NK) cells-mediated immune response comprising reducing the expression or activity of EAT-2 protein in said NK cells.

22. The method of claim 21, wherein said enhancing of NK cells-mediated immune response increases the ability of NK cells to kill target cells.

23. The method of claim 21, wherein said enhancing of NK cells-mediated immune response comprises inhibiting a biological activity of an EAT-2 protein in said NK cells.

24. The method of claim 22, wherein said increasing of the ability of NK cells to kill target cells comprises reducing a biological activity of an EAT-2 protein in said NK cells.

25. A method of reducing innate immune cell functions comprising increasing the expression of EAT-2 or stimulating a biological activity thereof in said innate immune cells.

26. (canceled)

27. The method of claim 25, wherein said innate immune cell is a NK cell.

28. The method of claim 25, wherein said innate immune cell is a dendritic cell.

29. The method of claim 25, wherein said innate immune cell is a macrophage.

30. (canceled)

31. (canceled)


SEQUENCE LISTING (17 sequences)

SEQ ID NO: 1 - protein, 132 aa, Mus musculus
MDLPYYHGCLTKRECEALLLKGGVDGNFLIRDSESVPGALCLCVSFKKLVYSYRIFREKHGYYRIETDAHTPRTIFPNLQELVSKYGKPGQGLVVHLSNPIMRNNLCQRGRRMELELNVYENTDKEYVDVLP

SEQ ID NO: 2 - protein, 132 aa, Mus musculus
MDLPYYHGCLTKRECEALLLKGGVDGNFLIRDSESVPGALCLCVSFKKLVYNYRIFREKNGYYRIETEPSTPKTIFPNLEELISKLKTPGQGMGVHLSNPIMRSGFCPGARRLNLEANVYENTDEEYVDVLP

SEQ ID NO: 3 - protein, 126 aa, Mus musculus
MDAVTVYHGKISRETGEKLLLATGLDGSYLLRDSESVPGVYCLCVLYQGYIYTYRVSQTETGSWSAETAPGVHKRFFRKVKNLISAFQKPDQGIVTPLQYPVEKSSGRGPQAPTGRRDSDICLNAP

SEQ ID NO: 4 - protein, 133 aa, Unknown
  Consensus sequence
MDXXXXYHGXXXXXXXEXLLLXXGXDGXXLXRDSESVPGXXCLCVXXXXXXYXYRXXXXXXGXXXXETXXXXXXXXFXXXXXLXSXXXXPXQGXXXXLXXPXXXXXXXXXXRXXXXXXXXXXXXDXXXXXXXP

SEQ ID NO: 5 - protein, 132 aa, Homo sapiens
MDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVKSNSDYVDVLP

SEQ ID NO: 6 - protein, 134 aa, Gallus gallus
MEFPFFHGKISRKACEDLLCKKGKNGSYLIRESETVAGALCLCVLFEKVIYTYRIFREYEGCYKVQTSEGVPQKVFKTLKDLIYNYEKPNQGLVTKLRYPVKKTSSSRRSQRFKTGNDDIYDEIDESDYVDVLS

SEQ ID NO: 7 - DNA, 2503 bp, Homo sapiens
gctgcttacacatctctcctcagagctgtcccttgacttgggggtgaatttcaggccaacagggcttcctgggatacaagagcgttctccatggatctgccttactaccatggacgtctgaccaagcaagactgtgagaccttgctgctcaaggaaggggtggatggcaactttcttttaagagacagcgagtcgataccaggagtcctgtgcctctgtgtctcgtttaaaaatattgtctacacataccgaatcttcagagagaaacacgggtattacaggatacagactgcagaaggttctccaaaacaggtctttccaagcctaaaggaactgatctccaaatttgaaaaaccaaatcaggggatggtggttcaccttttaaagccaataaagagaaccagccccagcttgagatggagaggattgaaattagagttggaaacatttgtgaacagtaacagcgattatgtggatgtcttgccttgaagataaggctgccggacaaagcaagttgaagagatgagtaacagttctcactgatgacccacttctgcaggcataggtccagagcaccaaactctagtggacaattcagactctcctggttgtgtaactgaagatgttctgcccaccagcaccagaggtcactctccacatccccgcctcccagacatataccaggagcaatttcaaaaccctctccagtttcactcttctttcttggaatgggacagcctgaacattttccctttgacttgttaaaggtaccaccctacatcatggttgacaccctcctttggaccatgcagtcagaggggcagctttatacagaggaggggcacacttgtctgtgagtttgaagccctgagttccagtcctgtggctgtgtgactttgaacacgtttctgcccatctccaggtcttagtttctttgtctctgtggttgggtgggatgataaacattgccccagtctctctgttgagcctgcttgtgtcaggtgaaggtgagggatcgggagtgatggagatgcatacagatcagcaccttctctgtacctgctggacccccatgtgcacccttctccctccagcctgggctactctcctcctggttctttctaatcttaaacttcctggtgagcattgccccagccacactttctccctggttttgcttaattccacttatgcatcagtcatttcaacagcatgaatgtggttctgtacagtgatgtatgtggagatttctaaaatattctatgtgtgatttctgttcccaactaggttctaagtgccatcaaagcacggttgggtctcccttgtatcttccacagtgtgtagaaatttgtgctgcccagagctgtgcctagttcatagaaactgactggaagcacattgctgagggaggtttgttgaattggtttttaaggtttactgaaattgatttgctgaatttttctgctagttcaaaatgtgaattaggacctggtcagtttgaaatataccaaattctatgccctttctcttacctatcatcaaattgtagtaatgtatttcaccccactggacttatcttcagagttttaaaagagaggagctctcgacttagaggtaatatgaacagatgaacagacactgtggctggagccccaaagtgtggagcattgtgagatttggggtcacagcaatttatggctactattccctgggtctggtaggtaggacaaatgtcctcttttacttttcaaaactggcttaggtattcatggaccttgattcttctatatacattttataataaatacgtgaaattgcttaaaacctctttttggaatttttaatgaacttgcaacaaatttgtaaataggagagaactgacgttcttgtgatgttttcccatgcataaacatgccacatccatgttcttgggtcatcttttttgccctttcatggagtcataaattttcccaactgaagtcttgtatattctgttagattaattcctatttctagttgctgtaaatgatatcttatgttttattacatttctaataggatatggtgttggtgaaatgttaaccctctcattagatctactagtttacctgttgattctattgtgttttctatgtaaatgattttgtcagctataaataataacattttattttctctttccttgcaatacctatgctcatttattttttatgtttacccattggttagggcctcctgtatacattaaacagttcatagtcatgatagtgagtattcttacactgttcccagtattacagggaatgcttagaaattttctttatttaaacattatgtttgttgtagccttgttaaaggctattttataatttttactagaaatattttgacatttattgtgatttttttctatctctaatctattgagatagtcacattccttttgtcttcactccattataaaggtaagttaccttaataaaattgttgatatcatttcatc

SEQ ID NO: 8 - DNA, 18041 bp, Homo sapiens
aaaggaacaatgcttgagggtgttcaaatggccttttttcaccagtggctgggagccttagccatggaatgggctgtcctcatccacagtgtcccactgttttttattgccattaatgtcttccatttttagtgtcatttgatccacggaaatcccaccctttcaattttcccattgtatctgagcatatcaattctctcagcatccaaatggtttttgagcacccagtctaagctaagcatagcccaatcctaggcaacttcagcagaaaatgggtaccattacagcaaaagcagcggtgtgcttcctggaaaggataccagtagccaaagaagttgatgatagtccatggtaatatacaaggctgcaacacagttccaggaagcttgacttcagcattgcctccctagccatgagcatcactgattccattgtgtgtttaaatgtgctttacctacttctgtgagtcacatgtgggccatttagagggtgaaaagtgttggtcattttatcaccccgccctcctcatttcctgaatcttgtgaagctggctgagtcccacagaccgctagaggaagaggtagctccacaggaggtacagctgcttacacatctctcctcagagctgtcccttgacttgggggtgaatttcaggccaacaggscttcctgggatacargagcgttctccatggatctgccttactaccatggacgtctgaccaagcaagactgtgagaccttgctgctcaaggaaggggtggatggcaactttcttttaagagacagcgagtcgataccaggagtcctgtgcctctgtgtctcgtgagtagcctcatcttgcacacatctgacatcgtatgttgaatgtggcaatggctgccacacagtacaggaattcatatccctcagtatttcactctgactttggagtctcaatgagtagaatgagaagtaatggacactgccaggagaggaatgtatctgtccatgctcctccatctgcctgctctccctggtgtaaaggccctgcaagcagactctgcagcagcaggagggacatggtggtggtgaagggaagggctgcaggaagtacaggggtacagagggatggaaagaaggtacaaatgtcagaggtcctgtcctccagagatctgtttatttggtatggaaaaggctaggggttcttcaagaaatttcaatctgaaasacttattctgcagcatctgcttgcaatacgtaaactataaatacaatcatatttcacattttgttagatttgtgaaggacttcaatattcgtcatttcctttcatcctagtagtctatcaaggcaagcataagcagtcaccatttcataggtaaagaaacagagacctgaatttctcaaataatttgtttgtgttaaaactcatttggaatttccagagtctaagttaaacatgaaattccaggctgggcatggtggctcatgcctgtaatcccaacactttgggaggctgaggcaggtgaatcacttgaggtcaggagtttgagaccagcctggccaacacggcaaaaccccatctctaccaaaaatacaaaaattaacctggtgtggtggcgtgtgcctgtaatactggctacttgagaggctgagacaggagaatcacttgaacccaggaggcagaggctgcagtgagctgagattgcaccactgcactccagcctgggcaatagagtgagactctgtccaaaaaaaagaaaaaagagtggctgggcacagtggctcactcctgtaatcccagcactttgggaagcggaggcaggtgaatcacctgaggtcgggagttcgagaccagcctgaccaacacgaagaaaccccrtctctactaaaaatacaaaattagccaggtgtggtggcgtatgcttgtaatcccagctatttgggaggctgaggcaggagaatcacttgaacccgggtggtggaggttgcagtgagctgagattgtgccattgcactccagcctgggcaacaagagcaaaactccatctcaaaaaaaaaaaaaaaaagaaaaagaaaaacaaattccaggccacagtatcaaagagacctagctgaaggaacttgtttatgcccagtttagaaagcaatcytgaacagaaatcactagaaacctcctatctttatgtatgttcctctttgtaaagtagggaccactgaaccttttattatacctgtaacagaaattaaaatccaaatgtctcttgttaaagaaaaaaattactcaatgtcacttgttaaattatggttgggaagactttattcaggatcatcacaaaggtacagggaccactgcaatgagctcttgcggcacagaaaagagactggactcaactctgaatccagcatggacaagtgggaatttatggccaaggagcagtgtggctgtcrgtagacagaaaattactcggaggaaacatcaggggtaaagtggattctggctaaaccagcctaacagaattgtagctgaagacaggccmgggtgatcagatgttgcctgggggatggtggaggatgaggaatctgatcagaatccaacagtgatcagatagctagggtgagrggtccttgccaaactgacttagtagggttctttgctaaaactggattttacaaggaagtacacaaatgggccttggaataggctcagaagtctgattatagacaaaagtgattgtcattactaatagaatacacagggacatatttctttctttaaatggtactggagatacaggctatttctctcatatctacatccaacagtaccatttaaagaaaaatatatattggccaggcgcggtggctcatgcctgtaatcctagcaatttgggaggctgaggtgggctgatcactgaggtcaggagttcaagaccagcctggccaacataatgaaacccccatctactaaaaatacaaaaattagccaggcatrgtgggtgcagttgtatcccagctacttgggaggctgaggcatgagaatcgcttgaacccaggaggcagaggttgcagtgagccgagatcgcgccattatactccagcctgagagacagagaaagactctccatctcaaataaaataaacaaagagtggawaagatctgcttacatgtaaatgggatggagggcattttagraaggggaggaggtaggaacaggagtctagaagtgggatattttaaaggatttacatacaaggttcctaaaatgtagtttggaaaccagcttttggaaaaggagggtgggataagatcatggagttttaaaagaatggttctatagataacagggaaacatggatggtttctgagcagggaagtgaaaagactggtgaatttaggaacttcaatttgacgtggagaacacaatgaagctgggaaatgatggcaacagaaactgttgtgtctctggacgggagacggtgaagagctagttgtgctggggcaagcagtgggaagggaaaaaacaagaaggtgcaaggacagacmttggccaggatctcagggtatatggtcaatatgggattctcttccttyctttctttcttttttttttttttagacagggtctcactccagctcctaggctggagtgcagtggtgcgatcatggctcactgcaacctcaaccttctgggttcaagagattctcctgtctcaggccccctgagtatctgggactacaggtgcrcactaccatgcctggttaatgtttaaattttttgtagacatggggtctcgctatgttgcccaggctgatttcgagctcctgggctccagtgatccacccacctcagcctcccaaagtgctgggattacaggcatgagccactgcacccagcctatattgggattctctttgaaaattgatatttattctcaactgttctgtggtgcatcagcccatcttgcatattcatctaaagcaaaggtcactaagagatgctaaaaaatgggccaggcgtggtggctcacacctgtattcccagcactytgggaggccgaggcgggcagatcactaggtcaagagattgagaccatcctggccaacatggtgaaactctgtctctactaaaaatacaaaamtaattagctgggtgtggtggcatgygcctgtagtcccagctactcaggaggctgaggcaggagaattgcttgaacccaggaggcggaggttgcagtgagccaagatcgcaccactgcactccagccgggttacagagcgagacttgatctcaaaaaaaaaaaaaaaaagaagagaaaaaaagagaagctaaaaaatgaaaataaaccaagtggagaggcaaaaataaatatttgttaagcccctgcacatgatcaggtctcacccacctccctacacatgtcactgaaaggcaaacaacatttcattcaccctcaggcttaaggcttaggtcaagtgacccctgaagctatctcctcatagggccttgtaaaaatgtattatcatacagataagtatggactattaaattctagttgttcaacaaatatttgaattctctcatatgtcagacagtgtgctagacactcggttgatgccttcaacacaactcagagcctagtggggaaaataaagaagtaaacagacacagtgatgcatgtgataattatgatgattggactatcattagggcactgtagagctgacagcaaaaaggggagaaccagatattggagctgagactttaggagaataagtggaaccttcattcattcaacaagcatatactgagcacttattatgttccatgcgtggtcctaagcattggtaatacagtgataaatactactggcaatgtttctgccctcatgaagttaatttcagtaagaggagaaaatatgatgaaaatgaaacagagtcatrtgacagggagagacacagctgctgctttccattgggtacaggaaaggtttctctgaagaggtcacatcaaccaattcatgcttcaagatacaaccctttgcaagcaaagcaagaattactattatgatacaagtgttagcctgcttgtcttcactgacacagaaaaagttaccatgtaggtattatctatgagccaaacattttgctaagcatggtacatgcattatcttatttgtcatttgcaatccctttgcrgtaggcgacatgatcactattttgtgagaaaataaactgtagcttcttatttaatagcttgcctagggccaarttgctaatatgttattacattccctctcttaccaatgtccccagcttcactgaaatatgtttcaggtaatcaccttggatctgtgttataaaatgcccaagcaggggacctgagggcttgggagtggactctgatatgcagtgtccccttcttctctgctgcattaaaatgggtctcccctggacacccagctttaaaatttctctgttttgtactctttcyctttatttcttagactggccgacacttagggagaatagaaaagaacctacgttgaaatattgggggctggttcccccgatacttcaggactttcacagagctaaatatttagtgatgaaaatactgtgaggaaggttagaaataaaagaaggaggtcacagagagagctgacctagcagcatatggcactaaccagaaagctatgtggtaactgtggacaggaaatgttttctgatcttacaatgtaatagagtgtctagaaaactccagtgtgtaaaaggttgtctgtgtagtgtgactttgtgagtgtctaaactgtctgcctttatcatcagtaaaattaaatggagtagatattaggaggtgcaaaggattcatctgcaagtgtgaagaaaaggaggactttgcaaagccataggaaggctgcaggtgaggaagacctgagccctaacctactgcatgctctcattaagaataataatggcaatgtctatctattgccttgagagtcatttatgagtctcagattctgagacttttgcaaggattgtagagtacagaaagaaaagaaatgatccattttgttgtggtggtttatcaagctaatgaaaattcattttagaaaatcaaatatagagaaaggtataaaaaacaaaacaaactatatcagtctcaccactctgaaatcaccatttttaatattttggcatacatctctccagacttctstgcgcatctataaaccaatatatgtacataaatggttcattatacatgatsgcttgcaacctgttttctccataattgtaggtctctttccccatcaaagaaagcaggtcatccccttatgactgcttagtattttattgatttgatatatcataatgtatacatatcatacattaggttattttcaattttcatttgcaatttagtccagtggactcagtgtaatttggaatgattaacatttttgtacttacatctgtgaaagtttgccttactccctagttaggataaaatcctagatgtttaaaaaaatcttcatgcacactgaaaaattagtaccacaaaaggttgcaccaagatacactgagatcaggarcagtgataaggccagaggtcaaaagtcttaaaatatttctttgttgacttggatttcccacttgcatcatggcatggaaaaatgcagaacacattgttgatattatagggtgcagtcgattccatttccttcagctgaccttttgaaggttagttaaaataaaaacttgaacacaaacaagtttccccacaacaatggccacaacatgagaccgtgctaatctgtgggctgttcatgcgttgagcaaggactcagagatccgtgcacagcacctcatggaacgatcactttgaaagctaaatagatagagtaaaacaattcaaagatatgggaaaacttagaggtcctgtattccagcctttttgaacctcaactgagaaattaatgcctaagatttttaaatgwtttttataagtccagtgtatttctcataaatgacgacaaygatttccatagaataaacaaaaaggtaggctccactgggtatcagtctcacacagagagtcaagacaccacatatttattgagaacataactgcaaaaataggaaacactttttctatctgaggccaaaaaccaatcatgaaaacagcaaaagcacatatgtataaggaattaaaatttagtttatatattagaaagataaattatattcatctacctttttatgccatgaatatgtcttactgctttattgaggtatcactgacataaaataaaaagtgtacaatttgatggattttgacatatgtatgtacacttatgaaacgaacacagccaagataatgaacatatttatcacctccaaaacttcacttgcacccctttgtaatttctcatcaacatacctcccacctatgcccagactctgggccaccacaaactgctttctggcactatagattaatgtgtatttttccagaatttaatgtaaatcaaatcatagagtatgtagtcatttttatctgacttcttttattcggaataattattttgagatccatctatgtggttgcatgcagcaatagttcatttttttgcattgctgaatagtgctccattktagggatgcatcccaaattgcttactcatttatagctattacarctaaagttgctatgaatatttgtgactaagtctttgtgtgggtgtatgctttcatttctcctgggtaaataatcaggattggaacagctaagttgtgtgttctatgtaggcttatcttttttaagaaactgccaaactgtttyccaaagtgatcatgcgatctcactattcccatgggcattacatgagttctgattgctctacatccttaccaacatgtggaacggtcagtcttttacattttaactgtattaataagtgtgtagtggtatctcattgtgagttgaatctgcgtttcactaatgactaatgatactgggtatctttacgagtgcttatttgccatccatgtattttcttttctttttttattgaggttcaacagcagattttactttttttatttttaaaaacttaagtctaattcaagtaaaataagctgtacatatttaaagtatacaatctaattcattttattttatttgatagataaaattatatgtacttattttgtacaatgtgatgttttaaaatatatatacgttgtggaatgactaaatctagctaattaacatatgcattacctcatcaagttatcatttttgtagtgagaaaacttaaaatatcctctcagcatttttcaaaaatataatatatggttattattttattttattttttttgagatggagtctcactctgtcacccaggctggagtgcagtggcaccatctcggctcactgcagcctccacctcctgtgttcaagctattctcctgtctcagccgcctgagtacttgggattacaggtgcctgccaccacacccagcaaatttttgtatttttagtagagacagggtttcaccatgttggccaggctggtcttgaattcctgaccttaggtgatccacccaccttggcctcccaaagtgctaggattataggcctgagccactgtgccctgcctaagaacgtaatatatggttattaactatagtcatcatgttgtacaatagatctcttgtttaactgaaattttgtatcctttgaccaatatctccstaatyctccccctctcactgacaaggcctgagaactctcattctactctttagttttatgatatcaactttttttagatgccacgtatgagtgagaccatgtggtatttttctttctgtgcctggcttatttcagttaggataacatcctccagatttatccatgttgttgcaagtaacaggattttatttttctatggctgaataatattccattgtgtgtgtgtgtgtgtgtgtgtgtgtgtgtgtgtgtgtgtaactttcttcattcatctattaatcaagttaggttgattccatgtcttggctattgtgactaatattgcaataaaagtgggagtgcagatatttcttcaacatactagattcatttccattggatatatacacagtagtgaggttatatagtaattacatatatatatatatatatatatatatatatwtwttttttttttttttttttttttcagacagagtctcgctctgtcgcccaggctggagtgcagtggcatgatcttggctcactgcaacctccgcctcctgggttcaagcgattctcgtgcctcagcctctcgagtagctgggattacaggcatacgccaccacacccagctaatttttgtatttttagtagaracgtggtttcactatgttggccaggctggtctgaaactcctgacctcgtgatctgccctcctcagcctcccaaagtgttgggattacaggcgtgagccacggcacccggccagtaattctatttttaattttttgaggaacctccatactgttttccatatggctatactaatttacattcccatcagcattgtacaagggttttcctttctctatatcctttgtcttttttgataatagccattctaacgggcatgatggtgggtgcctgtaatcccagctacgcaggaggctgaggcaggagaatcgyttgaacccgggaggcggaggttgcagtgagcygagatcgcaccactgcactccagcctgggcgacagagtgatattccgtcycaraaaaaaaaaaagagtttttttaggattgtagattaacaaaggtattgcatatgaaacataacgatagatgttacctggatttgcatagaacattttgattcacacagtcattttttccacaggtttaaaaatattgtctacacataccgaatcttcagagagaaacacgggtattacaggatacaggtaagaacaaaaaggacgacaacaacaacaacacaaaagttgggggagaagaacctgggaacatgtcttttacctgagccatgactgtgcgaaagcattctaaaaaggaaagcacaatgcagagatgagagtcagatcgagttaacctggtttttccctgaagagaaataggaaggttctaggcttgggttctactttttttgttgttgttttgtttttttyagacggagtctcgctctgtcgyccaggctggagtgcagtggtgcgatctyggctcactgcaacctccgcctcccgggttcaagcgattctcctgcctcagcccccgcccccccaagtagctgggactacaggcgcgcgccaccamccccagctaatttttgtattttcagtagagaagggttttcgccgtgttagacaggatggtctcgatctcctgaccttgtgatccgccggcctcagcctcccagactgctgggattacaggcgtgagccaccgcacccagctgggttctacttttgaataaccctcactgttattccactgtggctcctccactggccaaacacaccagcctggactccaagcaaaacctccgttgaattagaagactcaacaatgatgcaagatggagctagaaagggacggtggaaagagktcagagtgtttagggagtcaagtgtgcccaatagttgtacgaaaatatagtgggaagcatcagatagctgaaagtcttagggtttgtttaatttttctggtttactgagagctaagcagaaaaacattttgcttcttttaaatttagttctctggcttaagaagtagagtataataaacataactgctaacagagaatcctaaaataatctttctcaaacacttacatataaggttaaggagactacagatttctaagaatcttcagtatattaatgtttactctgaatatctgaaagagactagaatataaaatgatccccaaacacctcacttatactatttttcttttgctatagaggacattattgaggcagttgacaaaatttgaataaattcaaattttgaataagattaacaaatagtactgtatccaggttaatttcctgattttaatcattgtagtatggttrtgtaatatccttacttttgaagaaatactgaagtatttaaagggatattatgtctgcaaattaactttgaaaatagcttatatatatgtgtatatatacatgcacaatatatgtgtatatatttggaaagagaataaaaagcaaatataataaaatgttaacattggggtgtgggtgaaatatatggtaatttttaaaaattatttttgcaactttgctatgtctgaaataatttcaagataaaatgtcttataaaaagcaacttattttaagacttttttttctgtttattctttgggaaattctggataattcctcagtaccctgttttgtctcttgtgtggacaaaatagagggagacatccaaatcatttgtaactcagacacaaagaggcgtttcactggggagcagctttgagcttagaagcctgcagtgatgccttttgttgaaatgactgcattaaaggaacctccttgcagatgactttgatgtcagcctgtacctctaatacagaggttaatcaggcgcatatcagaaaaccttctgatcaccaacccctttctctagtctgacttggtggccatatgaaaaccacagtgacgatcaagaaaaccaaatacacaaaagtagaggaagaaaattcatgtgtgtgtgggtgtgtaagacagagtcttgctgtcacccagactggagtgcagtggcgtgatctcggctcactgcaacttccgcgtcctggttcaagcgattctcctggctcaacctcccgagtagctgggattacaggcgtctgctaccatgcccagctaattttttgtatttttagtagagacggagttttgccatgttgaccaggctggtctcaaactcctgacctcaagtgatccacccacctyggcctcccaaagggctgggattacaggcatgagccactgagcctagccagaaaattcttttcattttaaaattatttytaaaaggttatattagaaaatagtgcttggtgggccaggcatggtggctcccgcctgtaatcccagtactttgggaggctgaggcaggcagatcatgaggtcaggagatcgagacyatcctggctaacacagtgaaaccccatctctactaaaaatacaaaaaaaaaaaaaaaaaaaattagccaggtatggtgatggtgcttgtaatcccggctacttgggaggctgaggcaggagaatggcatgaacccgggaggcggaacttgcagtgagctgagatcacaccactgcactccagcctgggccacagagtgagactccgtctcaaaaaaaaaaaaaaaaaaaaaaagtacgtggtgtatagctggtaaacagaaaacatctatttacaacttaactgagttgtgtwgccattcccactttctatttacattgggacttttttggttttgttttgcccacatggagattgtgaattcatcaagtatacaaattgtgtattttaaattcataaagtatacaaawtgcatattttaaagacatggaatcaacctaaatgcccatcagtgatagactggataaataaaatgtggtacatatacaccatggaatactaagcagcctaactgttctccaaatttgaaaaaccaaatcagaggctggtggttcaccttttaaagccaataaagagaaccagccccagcttgagatggagaggatcgaaattagagttggaagcatttatggtaaaatctctcacatggggatcagaaaggagcttaggtcaaaaaagaatgaaatcatgtcctttgcagggacatggatggagctggaggccattatgtttagcaaactaatgcaggaacagaaaaccaaatactgcatgttctcacttgtaagtgggagctgaatgataagaacacatggacacacagaggagaacaacacacacaggggcctgttggagggctcagggtgggaggagggagaggatcaggaagaatagctagtgaatgctgggcttgatacctgggtgatgggatggtctgtgcagcaaaccactacggcacacgtttacctatgtaacaaacctgtacatcctgcacatgtacccctgaacttaaaataacagttggaattttttttaattgcatttttgcattcccatcagaagtccacttccgatagtcccttaagatagcaaagcccgcctgtaggtacagacgtaagcccttacaggggtacccttcasagtgtatctgcgagaactgccccaggtaaacttsccaaggatggggttcacttgatactatggactgcgctgggatcccttgattgcaaatcctctttactcagagtggctcaagtcagaggttggtcttagaaatgagagtttctgtgaactgggagtggtgtgagctccagccacttgttttcagtttttcagctcatagaagggcagccctgaaaacccactgctttgagaagtgtcacatttcacacgcaattgcatgataatgagtgatttcctttcttgtatttttgcagactgcagaaggttctccaaaacaggtctttccaagcctaaaggaactgatctccaaatttgaaaaaccaaatcaggggatggtggttcaccttttaaagccaataaagagaaccagccccagcttgagatggagaggattgaaattagagttggaaacatttgtggtaaaatctctcacgtgggggtcagaaagcaatcttaatcctatttccaccaatgccttactttgtaaactcaatacagacaaatgctcttttctgtgacattatctctaaaaagcttcatcctactaagagcaatttccattttgttgagaagtgctgtcttttagtccctgagggccaagacagagagcttgctgcgtttgcagccaggatagcaagagtggttcctgggcagacagctgacttcaaattcattctgtgaccttgtgcagacgattcaagtttgagaataaatgggcaaatgggcagattttcaaatgagaaaatcactctagcccttcaagccctccccaaarcgtatactgttacactttgctcaatatttgctaagcattcatttataaagtgctgggtgtcaaattatgtcagatacatttttaaaggcattaagccgtagtggaaaatgagagatggtcagtaaatctgagtgcagagaatccctgcctaagcctctcaaattatatcatgagcagctactcttccttacccacactcttgaaatctgctctcagtaaagaatttactatcagttgcaatgttgtttgaatttagctcagacttccagaaacacttaggtcttaatcctgttttcccatgttggaatgtgtggttatattctcagtgaaagggatggcagttatcagtggcaggtgatgggatgagatcagatatcctccctaggagagggacagaggacagagagttctctgggagcctgcaatcactccatccccattctactctgtctgcggcaaagtcaccctttaggctgccatagccggcttctcatttccccagcaaggctaagcataaaatacaactcccttccctaccattgtgtccctgtccccccatccccatgaagggcagtgtgtactcatggacacaaaaaaatcactttctataggtccagctgcagatgtagtcccacagctactttcccccccaaatttatagaataaaaagtatacagtgtggcagatctagaaacccaggaaygtatctgtgctgcagcccttaggcaaacccagatgagttgctgggactggttggggtggtggtagcatttgtctcaacaatgagaaaagaatcataaatcccaggaggagaaccctcagaaatcttccccaagacctgggcatgaatctcactccatcagttttccatgccgggtcagtgctgacacagcaggctccaaaattctcagctactgaagaggaaatattccccatcaacctcttcctcccattcccattgcttccttagaagaagcttcagatgttcttactccaagggaggtgcagatttcttgctggtgaatggagacacttgtgctggaaggtctgaggcttggcttataaagagatctcctattttgttaggagggctccctgtagtaccccagcctttaaagatayaactttcaggtggcatcaacatggctctgtgcctggggaggggtcttctttcctttgagtggggttgtttgatatgacttctgggtgtggtctcatggctggtcttctgctttttttttccctccaaagaacagtaacagcgattatgtggatgtcttgccttgaagataaggctgccggacaaagcaagttgaagagatgagtaacagttctcactgatgacccacttctgcaggcataggtccagagcaccaaactctagtggacaattcagactctcctggttgtgtaactgaagatgttctgcccaccagcaccagaggtcactctccacatccccgcctcccagacatataccaggagcaatttcaaaaccctctccagtttcactcttctttcttggaatgggacagcctgaacattttccctttgacttgttaaaggtaccaccctacatcatggttgacaccctcctttggaccatgcagtcagaggggcagctttatacagaggaggggcacacttgtctgtgagtttgaagccctgagttccagtcctgtggctgtgtgactttgaacacgtttctgcccatctccaggtcttagtttctttgtctctgtggttgggtgggatgataaacattgccccagtctctctgttgagcctgcttgtgtcaggtgaaggtgagggatcgggagtgatggagatgcatacagatcagcaccttctctgtacctgctggacccccatgtgcacccttctccctccagcctgggctactctcctcctggttctttctaatcttaaacttcctggtgagcattgccccagccacactttctccctggttttgcttaattccacttatgcatcagtcatttcaacagcatgaatgtggttctgtacagtgatgtatgtggagatttctaaaatattctatgtgtgatttctgttcccaactaggttctaagtgccatcaaagcayggttgggtctcccttgtatcttccacagtgtgtagaaatttgtgctgcccagagctgtgcctagttcatagaaactgactggaagcacattgctgagggaggtttgttgaattggtttttaaggtttaytgaaattgatttgctgaatttttctgctagttcaaaatgtgaattaggacctgktcagtttgaaatataccaaattctatgccctttctcttacctatcatcaaattgtagtaatgtatttcaccccactggacttatcttcagagttttaaaagagaggagctctcgacttagaggtaatatgaacagatgaacagacwctgtggctggagccccaaagtgtggagcattgtgagatttggggtcacagcaatttatggctactattccctgggtctggtaggtaggacaaatgtcctcttttacttttcaaaactggcttaggtattcatggaccttgattcttctatatacattttataataaatacgtgaaattgcttamaacctctttttggaatttttaatgaacttgcaacaaatttgtaaataggagagaactgacgttcttgtgatgttttcycatgcataaacatgccacatccatgttcttgggtcatcttttttgccctttcatggagtcataaattttcccaactgaagtcttgtatattctgttagattaattcctatttctagttgctgtaaatgatatcttatgttttattacatttctaataggatatggtgttggtgaaatgttaaccctctcattagatctactagtttacctgttgattctattgtgttttctatgtaaatgattttgtcagctataaataataacattttattttctctttccttgcaatacctatgctcatttattttttatgtttacccattggttagggcctcctgtatacattaaacagttcatagtcatgatagtgagtattcttacactgttcccagtattacagggaatgcttagaaattttctttatttaaacattatgtttgttgtagccttgttaaaggctattttataatttttactagaaatattttgacatttattgtgatttttttctatctctaatctattgagatagtcacattccttttgtcttcactccattataaaggtaagttaccttaataaaattgttgatatcatttcatccttgcattgcttcacttgataatattttattatttttcataaactgtggaatttggtaagttaatatttcatttaagatttttgcattaaaactcataaaccaaataggactataatttactgaatcatggttacactgttcatataaagcgaagaaaagtctttttccttttaccattttctgcaacaactcttcaggtaaagattatttgttctgggctgggtgtggtggctcacacctgtaatcccagcactttgggaggccgaggtggggggatcacttgaggtcaggagctcaagatcagcctggccaacatggtgaaaccctgtctctacaaaaatacaaaaatgtagccaggtgtggtggcacacgcctataatcccagctacttaggaggatgaggtgggagaatcgcttgaacccgggaggtggaggttgcagtgagccaagatcatgtcagggcactccagcctgggtgacagagtgagaccctatctcaaaaaatatatatatagtaaataaataaataaaaataaacttatctgttccttgacattttttagtacagtactttcttataaaactgagtttgagactct

SEQ ID NO: 9 - DNA, 22 bp, Artificial Sequence
  Synthetic oligonucleotide
tgatgctcatactccaagaacg

SEQ ID NO: 10 - DNA, 21 bp, Artificial Sequence
  Synthetic oligonucleotide
aggcaagacgtccacatactc

SEQ ID NO: 11 - DNA, 20 bp, Artificial Sequence
  Synthetic oligonucleotide
aggatagagactgagcccag

SEQ ID NO: 12 - DNA, 21 bp, Artificial Sequence
  Synthetic oligonucleotide
aggcaagacgtccacatactc

SEQ ID NO: 13 - DNA, 22 bp, Artificial Sequence
  Synthetic oligonucleotide
acagaaacaggttcttggagtg

SEQ ID NO: 14 - DNA, 23 bp, Artificial Sequence
  Synthetic oligonucleotide
gcattcaggcagatatcagaatc

SEQ ID NO: 15 - DNA, 19 bp, Artificial Sequence
  Synthetic oligonucleotide
gggtggagccaaacgggtc

SEQ ID NO: 16 - DNA, 21 bp, Artificial sequence
  Synthetic oligonucleotide
ggagttgctgttgaagtcgca

SEQ ID NO: 17 - protein, 132 aa, Homo sapiens; identical to SEQ ID NO: 5